(19) United States
(12) Patent Application Publication
(10) Pub. No.: US 2023/0270960 A1
(43) Pub. Date: Aug. 31, 2023

(54) SYSTEMS AND METHODS FOR DELIVERY OF THERAPEUTIC GAS

(71) Applicant: Mallinckrodt Pharmaceuticals Ireland Limited, Dublin (IE)

(72) Inventors: Jaron M. Acker, Madison, WI (US); Jeff Milsap, Cambridge, WI (US); Robin Roehl, Janesville, WI (US); Jeffrey Schmidt, Fitchburg, WI (US); Craig R. Tolmie, Stoughton, WI (US)

(21) Appl. No.: 18/109,510

(22) Filed: Feb. 14, 2023

Related U.S. Application Data
(63) Continuation-in-part of application No. 17/984,893, filed on Nov. 10, 2022.
Application No. 17/984,893 is a continuation of application No. 16/748,319, filed on Jan. 21, 2020, now Pat. No. 11,529,479.
Application No. 16/748,319 is a continuation of application No. 14/709,316, filed on May 11, 2015, now Pat. No. 10,576,225.
(60) Provisional application No. 61/991,083, filed on May 9, 2014.
(60) Provisional application No. 61/991,028, filed on May 9, 2014.
(60) Provisional application No. 61/991,032, filed on May 9, 2014.

(51) Int. Cl. A61M 16/00; B63C 11/02; G01F 22/02; A61M 16/20; A61M 16/12; A61M 16/10

(52) U.S. Cl. CPC A61M 16/0051; B63C 11/02; G01F 22/02; A61M 16/204; A61M 16/024; A61M 16/12; A61M 16/1005; A61M 16/122; A61M 16/125; A61M 16/201; A61M 16/202; A61M 16/208; A61M 2205/6072; A61M 16/085

(57) ABSTRACT

A therapeutic gas delivery system with at least one gas supply subsystem is disclosed. The at least one gas supply subsystem may include a gas source coupling configured to receive a therapeutic gas source and form a fluid flow connection with the therapeutic gas source, a therapeutic gas delivery system controller, and one or more display(s) configured to be in communication over a communication path with the therapeutic gas delivery system controller. The display may be configured to display a graphical, illustrative, or numerical indicator of one or more flow or system parameters.

### CROSS-REFERENCE TO RELATED APPLICATIONS

This application is a continuation-in-part application of U.S. application Ser. No. 17/984,893, filed Nov. 10, 2022, which is a continuation of Ser. No. 16/748,319, filed Jan. 21, 2020, now U.S. Pat. No. 11,529,479, which is a continuation of U.S. application Ser. No. 14/709,316, filed May 11, 2015, now U.S. Pat. No. 10,576,225, which claims the benefit under 35 U.S.C. § 119(e) to U.S. Provisional Application No. 61/991,028, filed May 9, 2014, U.S. Provisional Application No. 61/991,032, filed May 9, 2014, and U.S. Provisional Application No. 61/991,083, filed May 9, 2014, the entire contents of which are incorporated herein by reference in their entirety.

### TECHNICAL FIELD

Systems and methods for managing delivery of a therapy gas from a gas source to a subject, and in particular to management of delivery of inhaled therapy gases, are described.

### BACKGROUND

Certain medical treatments include the use of therapy gases that are inhaled by the patient. Gas delivery systems are often utilized by hospitals to control the rate of therapy gas delivery to the patient in need thereof, to verify the correct type of gas and the correct concentration are being used. Gas delivery systems may also verify dosage information, patient information and therapy gas administration.

Known therapy gas delivery systems may include a computerized system for tracking patient information, including information regarding the type of gas therapy, concentration of therapy gas to be administered and dosage information for a particular patient. While these computerized systems may communicate with other components of the therapy gas delivery system, such as the valve on the gas source that controls the flow of gas to the computerized system and/or the ventilator for administration to the patient, such communication has not included an ability to determine the amount of treatment time left before the therapy gas remaining in the gas source falls below a predetermined minimum or the gas source is empty.

There is a need for a therapy gas delivery system that addresses at least the above.

### SUMMARY

Various embodiments are listed below. It will be understood that the embodiments listed below may be combined not only as listed, but in other suitable combinations in accordance with the spirit and scope of the invention.

A first embodiment relates to a therapeutic gas delivery system, comprising at least one gas supply subsystem comprising, a gas source coupling configured to receive a therapeutic gas source and form a fluid flow connection with the therapeutic gas source, a gas source valve adjacent to and in fluid communication with the gas source coupling, wherein the gas source valve is configured to have at least an open state and a closed state, a gas pressure sensor adjacent to and in fluid communication with the gas source valve, wherein the gas source valve provides a gas flow path from the gas source coupling to the gas pressure sensor, and the gas pressure sensor is configured to measure a gas pressure at the gas source coupling at least when the gas source valve is in an open state, to be in communication over a communication path with a therapeutic gas delivery system controller comprising a CPU, and to communicate a pressure value over the communication path to the therapeutic gas delivery system controller, and a therapeutic gas flow regulator down stream from the gas pressure sensor, gas source valve, and gas source coupling, and in fluid communication with the gas source coupling, gas source valve, and gas pressure sensor, one or more display(s) configured to be in communication over a communication path with the therapeutic gas delivery system controller, wherein the CPU of the therapeutic gas delivery system controller is configured to calculate a value for a run-time-to-empty from a volume value, a pressure value communicated from the gas pressure sensor, and an average therapeutic gas consumption rate calculated by the CPU from the gas flow rate value communicated from the therapeutic gas flow controller, and wherein the system display is configured to display the calculated run-time-to-empty value.

In a second embodiment, the therapeutic gas delivery system of the first embodiment may be modified to further comprise a therapeutic gas source having a volume and containing a therapeutic gas at an initial pressure within the volume, wherein the therapeutic gas source is configured to be operatively associated with the gas source coupling, and wherein the volume value of the therapeutic gas source is inputted to the therapeutic gas delivery system controller.

In a third embodiment, the therapeutic gas delivery system of the first and/or second embodiments may be modified to have the gas supply subsystem further comprise a therapeutic gas conduit having an interior volume that provides a gas flow path at least from the gas source coupling to the gas source valve, and a temperature sensor operatively associated with the therapeutic gas source or the therapeutic gas conduit, wherein the temperature sensor is configured to measure a temperature of the therapeutic gas source, the therapeutic gas conduit, or the therapeutic gas, to be in communication over a communication path with a therapeutic gas delivery system controller, and to communicate a temperature value over the communication path to the therapeutic gas delivery system controller.

In a fourth embodiment, the therapeutic gas delivery system of the first through third embodiments may be modified to have the gas supply subsystem further comprise a gas source identifier attached to the therapeutic gas source, wherein the gas source identifier contains information at least of the gas source volume and the identity of the therapeutic gas supplied by the therapeutic gas source, and a gas source identifier reader operatively associated with the therapeutic gas delivery system, and in communication over a communication path with the therapeutic gas delivery system controller, wherein the gas source identifier reader is configured to obtain identifying information from the gas source identifier when the therapeutic gas source is properly received by the gas source coupling, and communicate the identifying information to the therapeutic gas delivery system controller.

In a fifth embodiment, the therapeutic gas delivery system of the first through fourth embodiments may be modified in a manner wherein the therapeutic gas source is a compressed gas cylinder, and the gas source identifier is selected from the group consisting of RFID, a QR code, a bar code, or combinations thereof, which is affixed to an outer surface of the compressed gas cylinder.

In a sixth embodiment, the therapeutic gas delivery system of the first through fifth embodiments may be modified in a manner wherein the gas supply subsystem further comprises a therapeutic gas source detector operatively associated with the gas source coupling, wherein the therapeutic gas source detector is configured to detect when the therapeutic gas source is properly received by the gas source coupling, and communicate a signal of the presence of the therapeutic gas source to the therapeutic gas delivery system controller.

In a seventh embodiment, the therapeutic gas delivery system of the first through sixth embodiments may be modified in a manner wherein the therapeutic gas delivery system controller is configured to obtain identifying information from the gas source identifier when the therapeutic gas source detector detects the therapeutic gas source is properly received by the gas source coupling, and communicate a signal to the gas source valve adjacent to the gas source coupling to transition to an open state, and wherein the therapeutic gas flow regulator is configured to be in communication over a communication path with the therapeutic gas delivery system controller.

In an eighth embodiment, the therapeutic gas delivery system of the first through seventh embodiments, may be modified in a manner wherein the therapeutic gas delivery system controller is configured to obtain a gas pressure value communicated from the gas pressure sensor, and a gas flow rate value from a flow controller, and calculate a run-time-to-empty value for the therapeutic gas source.

In a ninth embodiment, the therapeutic gas delivery system of the first through eighth embodiments, may be modified in a manner wherein the therapeutic gas delivery system controller is configured to calculate the run-time-to-empty value for the therapeutic gas source from at least the gas pressure value, the temperature of the therapeutic gas source, the gas source volume, and an average therapeutic gas consumption rate.

In a tenth embodiment, the therapeutic gas delivery system of the first through ninth embodiments, may be modified in a manner wherein the therapeutic gas delivery system controller comprises hardware, software, firmware, or a combination thereof configured to perform a run-time-to-empty calculation.

In an eleventh embodiment, the therapeutic gas delivery system of the first through tenth embodiments, may be modified in a manner wherein at least one of the one or more display(s) is a status display that is configured to present at least the run-time-to-empty value.

In a twelfth embodiment, the therapeutic gas delivery system of the first through eleventh embodiments, may be modified in a manner wherein at least one of the one or more display(s) is a status display operatively associated with at least one gas supply subsystem that is configured to present a bar graph, a chart, a numerical display of a value, a visual alarm, identifying information from the gas source identifier, or a combination thereof.

In a thirteenth embodiment, the therapeutic gas delivery system of the first through twelfth embodiments, may be modified in a manner wherein at least one status display operatively associated with at least one gas supply subsystem is configured to provide a user interface that is configured to provide control of the therapeutic gas delivery system.

In a fourteenth embodiment, the therapeutic gas delivery system of the first through thirteenth embodiments, may be modified in a manner wherein the therapeutic gas delivery system controller is configured to include a residual gas pressure value in the calculation of the run-time-to-empty value for the therapeutic gas source.

In a fifteenth embodiment, the therapeutic gas delivery system of the first through fourteenth embodiments, may be modified in a manner wherein the gas supply subsystem further comprises a gas supply subsystem valve in between and in fluid communication with the gas source valve and the therapeutic gas flow regulator, wherein the gas supply subsystem valve is configured to maintain the therapeutic gas under pressure between the gas supply subsystem valve and the therapeutic gas flow regulator.

In a sixteenth embodiment, the therapeutic gas delivery system of the first through fifteenth embodiments, may be modified in a manner wherein the gas supply subsystem valve is a mechanically activated check valve configured to be opened by a cylinder being received, wherein the gas supply subsystem valve avoids sudden release of pressure and prevents air/O₂ from entering between the gas supply subsystem valve and the therapeutic gas flow regulator.

In a seventeenth embodiment, the therapeutic gas delivery system of the first through sixteenth embodiments, may be modified in a manner wherein the therapeutic gas delivery system comprises two or more gas supply subsystems, wherein the therapeutic gas delivery system controller is configured to calculate the run-time-to-empty value for each therapeutic gas source in each of the two or more gas supply subsystems, and wherein the therapeutic gas delivery system controller communicates a signal to the gas supply subsystem valve for the therapeutic gas source calculated to have the shortest run-time-to-empty value to transition to an open state.

In an eighteenth embodiment, the therapeutic gas delivery system of the first through seventeenth embodiments, may be modified in a manner wherein the therapeutic gas delivery system controller further comprises two or more subsystem controllers, wherein each of the two or more gas supply subsystems comprises one subsystem controller, and wherein each of the two or more gas supply subsystems is configured to be controlled by the two or more subsystem controllers.

In an nineteenth embodiment, the therapeutic gas delivery system of the first through eighteenth embodiments, may be modified in a manner wherein each of the two or more subsystem controllers is configured to operate the two or more gas supply subsystems to continue delivering the therapeutic gas if another of the two or more subsystem controllers fails.

In a twentieth embodiment, the therapeutic gas delivery system of the first through nineteenth embodiments may be modified in a manner which further comprises a primary delivery system, further comprising a first primary shut off valve, wherein the first primary shut off valve is down stream from the two or more gas supply subsystems, and in fluid communication with the therapeutic gas flow regulators and gas pressure sensors of the two or more gas supply subsystems, a first primary high flow control valve, wherein the first primary high flow control valve is downstream from and in fluid communication with the first primary shut off valve, a first primary delivery flow sensor, wherein the first primary delivery flow sensor is downstream from and in fluid communication with the first primary high flow control valve, and a first primary confirmatory flow sensor, wherein the first primary confirmatory flow sensor is downstream from and in fluid communication with the first primary delivery flow sensor.

In a twenty-first embodiment, the therapeutic gas delivery system of the first through twentieth embodiments may be modified in a manner wherein the primary delivery system further comprises a second primary shut off valve, wherein the second primary shut off valve is down stream from the two or more gas supply subsystems, and in fluid communication with the therapeutic gas flow regulators and gas pressure sensors of the two or more gas supply subsystems, a second primary high flow control valve, wherein the second primary high flow control valve is downstream from and in fluid communication with the second primary shut off valve, a second primary delivery flow sensor, wherein the second primary delivery flow sensor is downstream from and in fluid communication with the second primary high flow control valve, and a second primary confirmatory flow sensor, wherein the second primary confirmatory flow sensor is downstream from and in fluid communication with the second primary delivery flow sensor.

In a twenty-second embodiment, the therapeutic gas delivery system of the first through twenty-first embodiments may be modified in a manner wherein the first primary delivery flow sensor and the first primary confirmatory flow sensor are configured to measure a gas flow rate at least when the first primary shut off valve and first primary high flow control valve are in an open state, to be in communication over a communication path with a therapeutic gas delivery system controller, and to communicate a gas flow rate value over the communication path to the therapeutic gas delivery system controller, and wherein the therapeutic gas delivery system controller is configured to compare the gas flow rate value from the first primary delivery flow sensor to the gas flow rate value from the first primary confirmatory flow sensor, and determine the difference between the two gas flow rate values.

In a twenty-third embodiment, the therapeutic gas delivery system of the first through twenty-second embodiments may be modified in a manner wherein the primary delivery system is configured to provide therapeutic gas at a controlled flow rate to an injector module for wild stream blending with an air/O₂ flow stream from a respirator.

In a twenty-fourth embodiment, the therapeutic gas delivery system of the first through twenty-third embodiments may be modified in a manner wherein the first primary high flow control valve, first primary delivery flow sensor, and the first primary confirmatory flow sensor are configured to provide a feedback control loop to adjust the flow rate of therapeutic gas to the injector module.

In a twenty-fifth embodiment, the therapeutic gas delivery system of the first through twenty-fourth embodiments may be modified in a manner wherein the therapeutic gas delivery system controller is configured to adjust the first primary high flow control valve in response to a value received from the first primary delivery flow sensor to adjust the flow rate of a therapeutic gas to an intended value.

Another aspect of the present invention relates to an electronically controlled gas blending device.

A first embodiment of the electronically controlled gas blending device comprises a flow control channel in fluid communication with a therapeutic gas supply, wherein the flow control channel comprises at least one secondary subsystem flow control valve, wherein the at least one secondary subsystem flow control valve is configured to be in communication over a communication path with a therapeutic gas delivery system controller, and at least one secondary subsystem flow sensor, wherein the at least one secondary subsystem flow sensor is in fluid communication with the at least one secondary subsystem flow control valve, and the at least one secondary subsystem flow sensor is configured to be in communication over a communication path with a therapeutic gas delivery system controller, one or more inlets configured to connect to a gas supply, one or more inlet flow sensors in fluid communication with at least one of the one or more inlets, a blending junction in fluid communication with the one or more inlet flow sensors, and the blending junction is connected to and in fluid communication with the flow control channel, and a therapeutic gas delivery system controller configured to be in electrical communication with at least the secondary subsystem flow control valve and the at least one secondary subsystem flow sensor to form a feedback loop, and configured to receive a flow value from the at least one inlet flow sensors and calculate a flow rate of therapeutic gas through the at least one secondary subsystem flow sensor to provide an intended dose of therapeutic gas exiting the blending junction.

In a second embodiment, the electronically controlled gas blending device of the first embodiment may be modified in a manner wherein the at least one secondary subsystem flow control valve and the at least one secondary subsystem flow sensor are arranged in series along the flow control channel.

In a third embodiment, the electronically controlled gas blending device of the first and/or second embodiments may be modified in a manner which further comprises a secondary subsystem shut-off valve in fluid communication with the flow control channel, and wherein the secondary subsystem shut-off valve is configured to have at least an open state and a closed state, and to be in communication over a communication path with a therapeutic gas delivery system controller.

In a fourth embodiment, the electronically controlled gas blending device of the first through third embodiments may be modified in a manner wherein there are two or more inlet flow sensors in fluid communication with at least one of the one or more inlets, and the therapeutic gas delivery system controller is configured to receive a flow value from at least two of the two or more inlet flow sensors, and compare the two values to determine if the two or more inlet flow sensors are providing the same flow value.

In a fifth embodiment, the electronically controlled gas blending device of the first through fourth embodiments may be modified in a manner wherein there are two or more secondary subsystem flow sensors in fluid communication with the at least one secondary subsystem flow control valve, and the therapeutic gas delivery system controller is configured to receive a flow value from at least two of the two or more secondary subsystem flow sensors, and compare the two values to determine if the two or more secondary subsystem flow sensors are providing about the same flow value.

In a sixth embodiment, the electronically controlled gas blending device of the first through fifth embodiments may be modified in a manner wherein the therapeutic gas delivery system controller is configured to generate an alarm signal if the flow values from the two of the two or more secondary subsystem flow sensors are not about the same.

In a seventh embodiment, the electronically controlled gas blending device of the first through sixth embodiments may be modified in a manner wherein the two or more inlet flow sensors are arranged in series with each other.

In an eighth embodiment, the electronically controlled gas blending device of the first through seventh embodiments may be modified in a manner which further comprises an outlet pressure sensor in fluid communication with the blending junction, and configured to be in communication over a communication path with the therapeutic gas delivery system controller, and the outlet pressure sensor communicates pressure values to the therapeutic gas delivery system controller, and the therapeutic gas delivery system controller is configured to detect pressure fluctuations in the outlet pressure sensor.

In a ninth embodiment, the electronically controlled gas blending device of the first through eighth embodiments may be modified in a manner which further comprises a flow regulating valve between and in fluid communication with the flow control channel and the blending junction, wherein the flow regulating valve is configured to direct a flow of therapeutic gas to either the blending junction or to an outlet.

In a tenth embodiment, the electronically controlled gas blending device of the first through ninth embodiments may be modified in a manner which further comprises an over-pressure valve in fluid communication with the one or more inlet flow sensors and an external vent, wherein the over-pressure valve is configured to open at a predetermined pressure to avoid pressure surges from the one or more inlets to the one or more inlet flow sensors.

In an eleventh embodiment, the electronically controlled gas blending device of the first through tenth embodiments may be modified in a manner wherein the therapeutic gas delivery system controller is configured to receive a signal indicating a failure of another flow control channel and communicate a signal to the secondary subsystem shut-off valve to transition from a closed state to an open state.

Another aspect of the present invention relates to a first embodiment of a therapeutic gas delivery system, comprising at least one gas supply subsystem, at least one primary gas delivery subsystem comprising at least one primary flow control channel, at least one secondary gas delivery subsystem comprising at least one secondary flow control channel comprising a secondary subsystem flow sensor, wherein the secondary subsystem flow sensor is in fluid communication with the secondary subsystem flow control valve, and where the secondary subsystem flow sensor is configured to be in communication over a communication path with a therapeutic gas delivery system controller, and a secondary subsystem flow control valve, wherein the secondary subsystem flow control valve is in fluid communication with the secondary subsystem shut-off valve, and the secondary subsystem shut-off valve and secondary subsystem flow control valve are arranged in series, and a therapeutic gas delivery system controller is configured to be in electrical communication with at least the secondary subsystem flow control valve and the secondary subsystem flow sensor to form a feedback loop.

In a second embodiment, the therapeutic gas delivery system of the first embodiment may be modified in a manner wherein the at least one primary gas delivery subsystem is controlled by a primary gas delivery subsystem controller, and the at least one secondary gas delivery subsystem is controlled separately by a secondary gas delivery subsystem controller.

In a third embodiment, the therapeutic gas delivery system of the first and/or second embodiments may be modified in a manner wherein the secondary gas delivery subsystem comprises a secondary subsystem shut-off valve, wherein the secondary subsystem shut-off valve is in fluid communication with the therapeutic gas supply and the secondary subsystem flow control valve, and is configured to have at least an open state and a closed state; and the therapeutic gas delivery system controller is configured to receive a failure signal from the at least one primary gas delivery subsystem, and communicate a signal to the secondary subsystem shut-off valve to transition from a closed state to an open state if a failure signal is received.

In a fourth embodiment, the therapeutic gas delivery system of the first through third embodiments may be modified in a manner wherein the therapeutic gas delivery system controller comprises a primary gas delivery system controller and a secondary gas delivery system controller, and the secondary gas delivery system controller is configured to communicate a signal to the secondary subsystem shut-off valve to transition from a closed state to an open state to avoid interruption of therapeutic gas flow from the therapeutic gas supply to a patient without input from a user if a failure of the primary gas delivery system controller is detected.

In a fifth embodiment, the therapeutic gas delivery system of the first through fourth embodiments may be modified in a manner which further comprises an outlet in fluid communication with the at least one primary gas delivery subsystem and the at least one secondary gas delivery subsystem, wherein the at least one secondary gas delivery subsystem is configured to deliver a therapeutic gas to the outlet in the event of a failure of the at least one primary gas delivery subsystem.

In a sixth embodiment, the therapeutic gas delivery system of the first through fifth embodiments may be modified in a manner which further comprises a breathing circuit comprising an injector module, wherein the injector module is configured to be in fluid communication with a respirator and the outlet, and secondary gas delivery subsystem is configured to deliver a therapeutic gas to the injector module at the dose of the primary gas delivery subsystem to avoid sudden changes in the dose of therapeutic gas.

In a seventh embodiment, the therapeutic gas delivery system of the first through sixth embodiments may be modified in a manner wherein the at least one primary gas delivery subsystem and the at least one secondary gas delivery subsystem are configured to provide a flow of therapeutic gas in parallel.

In an eighth embodiment, the therapeutic gas delivery system of the first through seventh embodiments may be modified in a manner wherein the at least one secondary gas delivery subsystem further comprises a flow regulating valve between and in fluid communication with the secondary flow control channel and a blending junction, wherein the flow regulating valve is configured to direct a flow of therapeutic gas to a low pressure outlet concurrently with flow of the therapeutic gas to the outlet from the primary gas delivery subsystem.

In a ninth embodiment, the therapeutic gas delivery system of the first through eighth embodiments may be modified in a manner wherein the flow regulating valve is configured to automatically direct a flow of therapeutic gas to the outlet of the primary gas delivery system in the event the primary gas delivery system fails.

In a tenth embodiment, the therapeutic gas delivery system of the first through ninth embodiments may be modified in a manner which further comprises at least one display, wherein the therapeutic gas delivery system controller is configured to provide an alarm on the at least one display to alert a user to the failure.

In an eleventh embodiment, the therapeutic gas delivery system of the first through tenth embodiments may be modified in a manner wherein the therapeutic gas delivery system is configured to provide a regulated dose of therapeutic gas to the outlet utilizing only one functioning gas supply subsystem and only one functioning flow control channel.

Another aspect of the present invention relates to another embodiment of an electronically controlled gas blending device, comprising a flow control channel in fluid communication with a therapeutic gas supply, wherein the flow control channel comprises at least one secondary subsystem flow control valve, wherein the at least one flow control valve is configured to be in communication over a communication path with a therapeutic gas delivery system controller, and at least two secondary subsystem flow sensors, wherein the at least two secondary subsystem flow sensors are in fluid communication with the at least one secondary subsystem flow control valve, and the at least two secondary subsystem flow sensors are configured to be in communication over a communication path with a therapeutic gas delivery system controller, wherein the at least one secondary subsystem flow control valve and the at least two secondary subsystem flow sensors are arranged in series along the flow control channel, one or more low pressure inlets configured to connect to a gas supply, comprising O₂ and/or air from a wall source and/or pressurized cylinder, two or more inlet flow sensors in fluid communication with at least one of the one or more low pressure inlets, wherein the two or more inlet flow sensors are arranged in series with each other, a blending junction in fluid communication with the two or more inlet flow sensors, and the blending junction is connected to and in fluid communication with the flow control channel, and a therapeutic gas delivery system controller comprising hardware, software, firmware, or a combination thereof, configured to be in electrical communication with at least the secondary subsystem flow control valve and the at least one of the two or more secondary subsystem flow sensors to form a feedback loop, and configured to receive a flow value from at least one of the two or more inlet flow sensors and calculate a flow rate of therapeutic gas through the two or more secondary subsystem flow sensors to provide an intended dose of therapeutic gas exiting a third leg of the blending junction.

In a second embodiment, the electronically controlled gas blending device may be modified in a manner wherein an external gas supply is in fluid communication with one of the one or more inlets and provides a flow of air and/or oxygen (O₂) to the two or more flow sensors in fluid communication with the one or more inlets.

Another aspect of the present invention relates to a method of confirming the proper functioning of a therapeutic gas delivery system.

A first embodiment relates to a method of confirming the proper functioning of a therapeutic gas delivery system, comprising pressurizing a gas supply subsystem at least between a gas source connection valve and a closed shut off to a pressure above atmospheric pressure, monitoring the pressure between the gas source connection valve and the closed shut off valve with a gas pressure sensor, and presenting an alarm if the pressure between the gas source connection valve and the closed shut off valve decreases over the predetermined time period.

In a second embodiment, the method of confirming the proper functioning of a therapeutic gas delivery system of the first embodiment may be modified in a manner which further comprises which further comprises mating a therapeutic gas source to a gas source coupling, and opening a purge valve in fluid communication with the gas source connection valve, and between the closed shut off and the gas source connection valve to flush gas within the gas supply subsystem with gas from the mated therapeutic gas source.

In a third embodiment, the method of confirming the proper functioning of a therapeutic gas delivery system of the first and/or second embodiments may be modified in a manner which further comprises mating a therapeutic gas source to a gas source coupling, and opening the shut off to deliver a flow of therapeutic gas from the gas supply subsystem to at least one of the one or more flow control channels comprising at least one shut off valve, at least one delivery flow sensor, and at least one confirmatory flow sensor to purge the gas supply subsystem and the at least one of the one or more flow control channels.

In a fourth embodiment, the method of the first through third embodiments may be modified in a manner which further comprises reading a gas source identifier attached to the therapeutic gas source with a gas source identifier reader, wherein the gas source identifier contains information at least of the identity, expiration date, and the concentration of the therapeutic gas supplied by the therapeutic gas source.

In a fifth embodiment, the method of the first through fourth embodiments may be modified in a manner which further comprises selectively opening the shut off valve for one of the one or more flow control channels, while the shut off valve for each of any other of the one or more flow control channels is closed; and measuring the gas flow rate through the at least one delivery flow sensor, and the at least one confirmatory flow sensor of the one flow control channel.

In a sixth embodiment, the method of the first through fifth embodiments may be modified in a manner which further comprises sequentially opening the shut off valve for each of the other of the one or more flow control channels by selectively opening the shut off valve for the next flow control channel, and closing the shut off valve of the previous flow control channel.

In a seventh embodiment, the method of the first through sixth embodiments may be modified in a manner which further comprises comparing the gas flow rate through the at least one delivery flow sensor with the gas flow rate through the at least one confirmatory flow sensor of the one flow control channel; and presenting an alarm if there is a discrepancy between the gas flow rate through the at least one delivery flow sensor and the gas flow rate through the at least one confirmatory flow sensor.

Another aspect of the invention relates to a method of confirming the proper functioning of gas delivery subsystem and injection module operation.

A first embodiment relates to a method of confirming the proper functioning of gas delivery and injection module operation, comprising receiving an injection module at an outlet port, providing a flow of breathing gas at an inlet port at a breathing gas flow rate, wherein the inlet port is in fluid communication with the outlet port, measuring the breathing gas flow rate from the gas supply at a delivery flow sensor and at a confirmatory flow sensor, wherein the delivery flow sensor and the confirmatory flow sensor are in fluid communication with the inlet port and the outlet port, measuring the breathing gas flow rate from the gas supply at an injection module delivery flow sensor and an injection module confirmatory flow sensor, wherein the injection module delivery flow sensor and the injection module confirmatory flow sensor are in fluid communication with the outlet port, and determining if one of the breathing gas flow rates measured at the confirmatory flow sensor, the delivery flow sensor, the injection module confirmatory flow sensor, or the injection module delivery flow sensor differs from the other measured breathing gas flow rates by greater than a threshold amount.

In a second embodiment, the method of confirming the proper functioning of gas delivery and injection module operation of the first embodiment may be modified in a manner which further comprises providing an alarm if the breathing gas flow rates measured at the low pressure confirmatory flow sensor, the low pressure delivery flow sensor, the injection module confirmatory flow sensor, or the injection module delivery flow sensor differs from the other measured breathing gas flow rates by greater than a threshold amount.

In a third embodiment, the method of confirming the proper functioning of gas delivery and injection module operation of the first and/or second embodiments may be modified in a manner wherein the threshold amount is about 10%.

In a fourth embodiment, the method of confirming the proper functioning of gas delivery and injection module operation of the first through third embodiments may be modified in a manner wherein the low pressure delivery flow sensor and the low pressure confirmatory flow sensor are arranged in series, and wherein the injection module delivery flow sensor and the injection module confirmatory flow sensor are arranged in series.

In a fifth embodiment, the method of confirming the proper functioning of gas delivery and injection module operation of the first through fourth embodiments may be modified in a manner wherein the injection module delivery flow sensor and the injection module confirmatory flow sensor are bi-directional flow sensors that are configured to determine the direction of gas flow through the injection module.

In a sixth embodiment, the method of confirming the proper functioning of gas delivery and injection module operation of the first through fifth embodiments may be modified in a manner wherein the low pressure gas supply comprises a wall supply and/or a pressurized cylinder configured to provide air, oxygen, or a combination thereof.

In a seventh embodiment, the method of confirming the proper functioning of gas delivery and injection module operation of the first through sixth embodiments may be modified in a manner which further comprises providing a stream of therapeutic gas to the flow of breathing gas upstream from an output of the injection module, wherein the stream of therapeutic gas and breathing gas combine to provide an intended concentration of therapeutic gas.

In an eighth embodiment, the method of confirming the proper functioning of gas delivery and injection module operation of the first through seventh embodiments may be modified in a manner which further comprises connecting a sampling line down stream from the output of the injection module to sample at least a portion of the flow of gas exiting the injection module to a gas analyzer for measurement of at least the concentration of therapeutic gas, determining the concentration of therapeutic gas exiting the injection module, and comparing the measured concentration of therapeutic gas with the intended concentration of therapeutic gas.

In a ninth embodiment, the method of confirming the proper functioning of gas delivery and injection module operation of the first through eighth embodiments may be modified in a manner which further comprises adjusting a subsystem flow control valve to provide a stream of therapeutic gas at an intended therapeutic gas flow rate; and determining if the subsystem flow control valve is properly functioning, wherein the subsystem flow control valve is in fluid communication with the low pressure outlet port.

In a tenth embodiment, the method of confirming the proper functioning of gas delivery and injection module operation of the first through ninth embodiments may be modified in a manner which further comprises measuring the combined therapeutic gas flow rate and breathing gas flow rate at the injection module delivery flow sensor and the injection module confirmatory flow sensor, switching a flow regulating valve to divert the stream of therapeutic gas to an alternative flow path, wherein the flow regulating valve is up stream from and in fluid communication with the low pressure outlet port, and the subsystem flow control valve is upstream from and in fluid communication with the flow regulating valve, measuring the breathing gas flow rate at the injection module delivery flow sensor and the injection module confirmatory flow sensor, and determining if the flow regulating valve functioned properly by determining if the combined therapeutic gas flow rate and breathing gas flow rate decreased by the therapeutic gas flow rate when the flow regulating valve was switched to the alternative flow path.

In an eleventh embodiment, the method of confirming the proper functioning of gas delivery and injection module operation of the first through tenth embodiments may be modified in a manner which further comprises measuring a flow rate at two or more subsystem flow sensors, wherein the two or more subsystem flow sensors are upstream from and in fluid communication with the three-way valve; and comparing the flow rates measured at each of the two or more subsystem flow sensors to determine if the two or more subsystem flow sensors are in agreement.

In a twelfth embodiment, the method of confirming the proper functioning of gas delivery and injection module operation of the first through eleventh embodiments may be modified in a manner which further comprises calculating therapeutic gas blending ratio from the measured flow rate measured by at least one of the two or more subsystem flow sensors and from the breathing gas flow rate measured by the low pressure delivery flow sensor; and comparing the calculated therapeutic gas blending ratio to the measured concentration of therapeutic gas exiting the injection module.

In a thirteenth embodiment, the method of confirming the proper functioning of gas delivery and injection module operation of the first through twelfth embodiments may be modified in a manner which further comprises adjusting a subsystem flow control valve to be completely open to provide the stream of therapeutic gas at a maximum therapeutic gas flow rate.

Another aspect of the invention relates to a method to ensure the proper functioning of a therapeutic gas delivery system.

A first embodiment relates to a method to ensure the proper functioning of a therapeutic gas delivery system, comprising detecting a therapeutic gas source mated with a therapeutic gas supply subsystem, providing an initial purge of the therapeutic gas supply subsystem with gas from the therapeutic gas source, determining if the initial purge was successful, maintaining a shutoff valve down stream from the therapeutic gas source in a closed state, verifying that no flow is detected by one or more flow sensors down stream from the shutoff valve, and determining if flow is detected by one or more flow sensors down stream from the shutoff valve; and providing an alert if it is determined that the initial purge was not successful and/or if flow is detected by one or more flow sensors down stream from the shutoff valve.

In a second embodiment, the method to ensure the proper functioning of a therapeutic gas delivery system of the first embodiment may be modified in a manner which further comprises reading information associated with the therapeutic gas source to determine the identity, concentration, and/or expiration date of the therapeutic gas source, and verifying the therapeutic gas source mated with a therapeutic gas supply subsystem has the correct identity, concentration, and/or expiration date.

In a third embodiment, the method to ensure the proper functioning of a therapeutic gas delivery system of the first and/or second embodiments may be modified in a manner wherein the shutoff valve down stream from the therapeutic gas source in a closed state until the correct identity, concentration, and/or expiration date is verified.

The systems and methods may further comprise an alarm system to inform the user when a therapeutic gas source has reached a predetermined minimum run-time-to-empty. In various systems in which multiple therapy gas sources are engaged, the alarm system may also inform the user with a high-priority alarm when the total run-time-to-empty of the system has been reached.

Another aspect of the present invention relates to a therapeutic gas delivery system.

In a first embodiment, the therapeutic gas delivery system may include: at least one gas supply subsystem comprising; a gas source coupling configured to receive a therapeutic gas source and form a fluid flow connection with the therapeutic gas source; a therapeutic gas delivery system controller; and one or more display(s) configured to be in communication over a communication path with the therapeutic gas delivery system controller, wherein the one or more display(s) are configured to display a graphical, illustrative, or numerical indicator of at least one of a flow parameter and/or a system parameter.

In a second embodiment, the display may be configured to display a visual alarm.

In a third embodiment, the therapeutic gas delivery system controller may be configured to produce an audible alarm.

In a fourth embodiment, the display may be configured to display a bar graph, a chart, or a numerical display of a value of the at least one of the flow parameter and/or the system parameter.

In a fifth embodiment, the display may be configured to present a graphic of the therapeutic gas delivery system comprising indicators for flow of the therapeutic gas through the system.

In a sixth embodiment, the graphic of the therapeutic gas delivery system may include indicators for a status of the therapeutic gas source, a sample line, an injector module, expiratory tubing, an electronically controlled gas blending device, or combinations thereof.

In a seventh embodiment, the therapeutic gas may be nitric oxide.

In an eighth embodiment, the display may be configured to present a concentration and/or flow rate of nitric oxide and breathing gas through the system.

In a ninth embodiment, the therapeutic gas delivery system may include an electrical power supply connection, an internal battery, and a power button, wherein the power button includes a power indicator that visually indicates an electrical power supply status of the electrical power supply connection and a battery charge status of the internal battery.

In a tenth embodiment, one or more system parameters may include an electrical power supply status and a battery charge status.

In an eleventh embodiment, the therapeutic gas delivery system may include an alarm silence button, wherein the alarm silence button may include an alarm indicator that may visually indicate a status of one or more alarms of the therapeutic gas delivery system.

In a twelfth embodiment, the therapeutic gas delivery system may have a global status indicator, wherein the global status indicator visually may indicate one or more global statuses of the therapeutic gas delivery system.

In a thirteenth embodiment, one or more of the global statuses may include a delivery status and an alarm status.

In a fourteenth embodiment, the therapeutic gas delivery system may include an injector module outlet and a corresponding port indicator, wherein the port indicator may visually indicate a delivery status of the injector module outlet.

In a fifteenth embodiment, the therapeutic gas delivery system may have a secondary delivery subsystem outlet and a corresponding port indicator, wherein the port indicator may visually indicate a delivery status of the secondary delivery subsystem outlet.

In a sixteenth embodiment, the therapeutic gas source may include a first compressed gas cylinder, wherein the one or more flow or system parameters may include a first cylinder status, wherein the first cylinder status may visually indicate whether the first compressed gas cylinder is delivering therapeutic gas or is idle, wherein the first cylinder status may include a percentage of available therapeutic gas in the first compressed gas cylinder.

In a seventeenth embodiment, the first cylinder status may visually indicate if the first compressed gas cylinder is in a low priority alarm or a high priority alarm.

In an eighteenth embodiment, the therapeutic gas source may include a second compressed gas cylinder, wherein the one or more flow or system parameters may include a second cylinder status, wherein the second cylinder status may visually indicate whether the second compressed gas cylinder is delivering therapeutic gas or is idle, wherein the second cylinder status may include a percentage of available therapeutic gas in the second compressed gas cylinder.

In a nineteenth embodiment, the second cylinder status may visually indicate if the second compressed gas cylinder is in a low priority alarm or a high priority alarm.

In a twentieth embodiment, the one or more flow or system parameters may include at least one of a left cylinder status, right cylinder status, a patient line status, a sample gas sample line status, a delivery conduit status, an injector module status, a patient gas sample line status, and a breathing circuit expiratory limb status.

In a twenty-first embodiment, the one or more flow or system parameters may include at least one of a secondary delivery subsystem status and dose.

In a twenty-second embodiment, at least one gas supply system may include a secondary delivery subsystem, wherein the one or more displays may be configured to display the delivery of therapeutic gas through the secondary delivery subsystem.

In a twenty-third embodiment, the therapeutic gas delivery system may include a breathing circuit, wherein the one or more displays may be configured to display the delivery of therapeutic gas to the breathing circuit.

In a twenty-fourth embodiment, the breathing circuit may be a t-piece resuscitator circuit, an infant resuscitator circuit, or an infant t-piece resuscitator circuit.

In a twenty-fifth embodiment, at least one gas supply system may include a therapeutic gas delivery system, wherein the one or more displays may be configured to display the delivery of therapeutic gas through the therapeutic gas delivery system.

In a twenty-sixth embodiment, the therapeutic gas delivery system may include a breathing circuit, wherein the one or more displays may be configured to display the delivery of therapeutic gas to the breathing circuit.

In a twenty-seventh embodiment, the breathing circuit may be a conventional ventilator circuit, an anesthesia circuit, a spontaneous circuit, a nasal cannula circuit, a high frequency jet ventilation circuit, a high frequency oscillatory ventilator unfiltered circuit, a dual-limb transport ventilator circuit, or a single-limb transport ventilator circuit.

In a twenty-eighth embodiment, the breathing circuit may be a high frequency oscillatory ventilator filtered circuit, an infant flow circuit, a precision flow circuit, a continuous positive airway pressure system circuit, a high flow breathing circuit, a high flow infant circuit nasal cannula circuit, or a high flow nasal cannula oxygen therapy circuit.

Another aspect of the present disclosure relates to a therapeutic gas delivery system.

In a first embodiment the therapeutic gas delivery system may include: a first gas supply subsystem comprising a first compressed gas cylinder configured to supply therapeutic gas; a second gas supply subsystem comprising a second compressed gas cylinder configured to supply therapeutic gas; and a therapeutic gas delivery system controller, wherein the therapeutic gas delivery system receives therapeutic gas from the first compressed gas cylinder until the first compressed gas cylinder is depleted, wherein when the first compressed gas cylinder is depleted the therapeutic gas delivery system controller performs an automatic switchover such that the therapeutic gas delivery system receives therapeutic gas from the second compressed gas cylinder.

In a second embodiment, the automatic switchover may provide continuous uninterrupted delivery of therapeutic gas to a patient.

In a third embodiment, the therapeutic gas delivery system receiving therapeutic gas from the second compressed gas cylinder may provide time to disconnect the first compressed gas cylinder and connect a replacement compressed gas cylinder to the first gas supply subsystem.

In a fourth embodiment, the replacement compressed gas cylinder may include a cylinder concentration such that the cylinder concentration may be verified before the replacement compressed gas cylinder is connected.

In a fifth embodiment, the replacement compressed gas cylinder may include an expiration date such that the expiration date may be verified before the replacement compressed gas cylinder is connected.

In a sixth embodiment, the therapeutic gas delivery system may include at least one display in communication with the therapeutic gas delivery system controller, wherein the at least one display is configured to display a visual alarm when the first compressed gas cylinder is depleted.

In a seventh embodiment, the at least one display may be configured to display a visual alarm if a replacement compressed gas cylinder is not full with therapeutic gas.

In an eighth embodiment, the therapeutic gas delivery system controller may produce an audible alarm when the first compressed gas cylinder is depleted.

In a ninth embodiment, the therapeutic gas delivery system controller may produce an audible alarm if a replacement compressed gas cylinder is not full of therapeutic gas.

### BRIEF DESCRIPTION OF THE DRAWINGS

The features and advantages of the present invention will be more fully understood with reference to the following, detailed description when taken in conjunction with the accompanying figures, wherein:

FIG. 1 is an overview diagram of an exemplary therapeutic gas delivery system and patient breathing apparatus, in accordance with exemplary embodiments of the present invention;

FIG. 2 is a diagram of the exemplary therapeutic gas delivery system, in accordance with exemplary embodiments of the present invention;

FIG. 3 is a diagram of the portion of the exemplary therapeutic gas delivery system downstream of FIG. 2 and/or which couples to the patient breathing apparatus, in accordance with exemplary embodiments of the present invention;

FIGS. 4A-4C is a flow chart of an exemplary pre-use performance validation process, in accordance with exemplary embodiments of the present invention;

FIG. 5 is a diagram of an exemplary arrangement of a therapeutic gas delivery system for use during exemplary embodiments of a pre-use performance validation process;

FIG. 6 is a flow chart of an exemplary process for determining whether various sensors are properly calibrated, in accordance with exemplary embodiments of the present invention;

FIG. 7 illustrates one example of a therapeutic gas delivery system;

FIGS. 8A-8C illustrate examples of display graphics, which may be displayed on the therapeutic gas delivery system;

FIGS. 9A-9D illustrate examples of device icons, which may be displayed on the therapeutic gas delivery system;

FIGS. 10A-10B illustrate an exemplary embodiment of the secondary delivery subsystem operational flow;

FIGS. 11A-11C illustrate diagrams of example breathing circuits, which may be used with the secondary delivery subsystem;

FIG. 12 illustrates an exemplary embodiment of the therapeutic gas delivery system operational flow;

FIG. 13 illustrates an exemplary embodiment of flow tile information, which may be display on the therapeutic gas delivery system;

FIGS. 14A-14R illustrate diagrams of example breathing circuits, which may be used with the therapeutic gas delivery system;

FIGS. 15A-15H illustrate exemplary embodiments of a method for loading and/or replacing gas cylinders; and

FIG. 16 illustrates an example of a computing system.

### DETAILED DESCRIPTION

Several definitions that apply throughout the disclosure will now be presented:

The terms “therapeutic gas delivery system,” “therapeutic gas delivery device,” “NO delivery system,” “therapeutic gas system,” “NO gas delivery system,” “delivery system,” “gas delivery system,” and “EVOLVE” are used interchangeably in this disclosure.

The terms “backup delivery subsystem,” “secondary delivery subsystem,” “eblender,” and “eINOblender” are used interchangeably in this disclosure.

The terms “therapeutic gas source,” “cylinder,” “gas cylinder,” and “INOMAX cylinder” are used interchangeably in this disclosure.

The terms “nitric oxide,” “NO,” “therapeutic gas,” “therapeutic nitric oxide gas,” and “INOMAX” are used interchangeably in this disclosure.

The terms “delivery conduit,” “therapeutic gas delivery line,” “delivery line,” “delivery circuit,” and “INOMAX injector tube” are used interchangeably in this disclosure.

The terms “sample tee,” “sample-T,” and “gas sample tee” are used interchangeably in this disclosure.

The terms “humidifier outlet” and “humidifier water chamber outlet” are used interchangeably in this disclosure.

The terms “humidifier” and “humidifier control unit” are used interchangeably in this disclosure.

The terms “sample line inlet” and “patient gas sample input connection” are used interchangeably in this disclosure.

The terms “tiles” and “display graphics” are used interchangeably in this disclosure.

The term “breathing gas” means any gas delivered to a patient (e.g., air, O₂, therapeutic gas, or a mixture of therapeutic gas and air/O₂).

The terms “sample subsystem,” “sampling subsystem,” “sample system,” “sample gas subsystem,” “gas analyzing subsystem,” “gas analyzer subsystem,” “gas sampling subsystem,” “gas analyzer system,” gas analyzing system,” and “eINOcal” are used interchangeably in this disclosure.

In exemplary embodiments, systems and methods of the present invention provide enhanced safety improvements over current therapeutic gas delivery systems by at least enabling accurate and/or precise determination and/or usage of information indicative of the run-time-to-empty for the therapeutic gas source. In exemplary embodiments, a plurality of therapeutic gas sources may be affiliated with a therapeutic gas delivery system. Further, in at least some instances, the present invention may determine and/or use information indicative of the run-time-to-empty for a plurality of therapeutic gas sources affiliated with a therapeutic gas delivery system.

In exemplary embodiments, therapeutic gas delivery systems of the present invention may comprise at least one gas supply subsystem, at least one primary delivery subsystem, and/or at least one secondary delivery subsystem, wherein redundant systems and/or components provide parallel or supplemental data enabling cross verification of component operation, fallback functionality, and/or fail-safe protection of the patient and the system. In at least some embodiments, the present invention may provide simplified therapeutic gas delivery systems and methods of fail-safe protection and redundancy that may allow seamless transition to backup systems automatically, for example, without the need of extensive training of a user. Further, in exemplary embodiments, the present invention may mitigate risks associated with sudden termination of inhaled therapeutic gas delivery and/or incorrect delivery of therapy.

In one or more embodiments, therapeutic gas delivery systems of the present invention may comprise, amongst other things, at least one gas supply subsystem as well as at least one least one gas delivery subsystem. For example, therapeutic gas delivery systems of the present invention may comprise at least one gas supply subsystem and at least one delivery subsystem comprising at least one flow control channel, wherein the gas supply subsystem provides a first therapeutic gas source having a volume and/or containing a therapeutic gas at an initial pressure for delivery to a patient. For another example, therapeutic gas delivery systems of the present invention may comprise two or more gas supply subsystems, a primary delivery subsystem having at least one flow control channel comprising a plurality of valves and a plurality of flow sensors, and a secondary delivery subsystem having at least one flow control channel comprising a plurality of valves and a plurality of flow sensors, wherein the two or more gas supply subsystems provide a first therapeutic gas source having a volume and/or containing a therapeutic gas at an initial pressure for initial delivery of therapeutic gas to a patient, and at least a second therapeutic gas source having a volume and/or containing a therapeutic gas at an initial pressure for subsequent delivery of therapeutic gas to a patient when the pressure within the first therapeutic gas source falls below a predetermined, threshold value.

In various embodiments, the primary delivery subsystem and/or the secondary delivery subsystem control the flow rate of therapeutic gas to achieve the set dose being delivered to a patient in need of the therapeutic gas, and, in at least some instances, the therapeutic gas may be blended with air and/or oxygen before being received by the patient.

In exemplary embodiments, systems and methods may determine the length of time that a therapeutic gas source may continue delivering the therapeutic gas before having insufficient pressure/gas volume, also referred to as “run-time-to-empty”, for example, by calculating the volume and pressure of therapeutic gas available from the therapeutic gas source, for example by using the ideal gas law, and the rate at which the therapeutic gas is flowing from the therapeutic gas source. As used herein, “run-time-to-empty”, “RTE”, or the like means the estimated time a therapeutic gas source may continue to supply the therapeutic gas at a current flow rate until the pressure remaining in the therapeutic gas source reaches a threshold value at which the ability to control or maintain the flow rate may be affected.

In one or more embodiments, a therapeutic gas delivery system comprising two or more therapeutic gas sources may first supply therapeutic gas from the therapeutic gas source having the shorter run-time-to-empty value and/or minimum run-time pressure. In various embodiments, the therapeutic gas delivery system may seamlessly transition from a first therapeutic gas source to a second therapeutic gas source when the first therapeutic gas source has reached the intended run-time-to-empty value and/or minimum run-time pressure. For example, systems and methods may enable source gas cut-over (e.g., seamless transition) between at least two source gases (e.g., therapeutic gas being received for delivery from one gas source may be halted such that the therapeutic gas may be received for delivery from another gas source) when run-time-to-empty for a therapeutic gas source is below a minimum threshold and/or when desired. In one or more embodiments, cut-over may be accomplished without any interruption of therapeutic gas flow, where cut-over may involve controller actuated opening of a flow path to a subsequent therapeutic gas source before closing, immediately after closing, and/or in parallel with closing the flow path to the initial therapeutic gas source to avoid sudden interruption of gas inhalation therapy, which may also be referred to as “seamless transition.” In at least some embodiments, usage of the therapeutic gas source may not be allowed if the source does not have a minimum run-time pressure (e.g., pressure below 300 psi, not enough pressure to perform purges, pressure low or waning indicative of leak, etc.).

In various embodiments, the therapeutic gas source having the shorter run-time-to-empty value is used first to provide sufficient time to replace the exhausted therapeutic gas source before the second therapeutic gas source may become exhausted. In various embodiments, a user may be alerted to the run-time-to-empty value, a need to switch over to another therapeutic gas source, and/or the need to replace an effectively empty therapeutic gas source, for example, after switch-over to a second therapeutic gas source provided as a backup to avoid sudden discontinuation of the therapeutic inhalation therapy. In embodiments wherein the therapeutic gas delivery system is configured to engage multiple therapeutic gas sources, the program or algorithm incorporates the number of therapeutic gas sources connected to the system into the run-time-to-empty calculation. For example, run-time-to empty is calculated in the manner described above for each connected therapeutic gas source and the program or algorithm uses this data to calculate a total run-time-to-empty for the therapeutic gas delivery system for use of each therapeutic gas source sequentially. Sequential use of multiple therapeutic gas sources connected to the therapeutic gas delivery system means that a first therapeutic gas source is in fluid communication with the therapeutic gas supply system and at least a second therapeutic gas source is connected to another therapeutic gas supply system, but is shut off from fluid communication to one or more therapeutic gas delivery system(s).

Principles and embodiments or the present invention also relate to algorithms to obtain values from sensor(s), valve(s), regulator(s), and/or detector(s), and perform the calculations of run-time-to-empty based on the obtained values. In various embodiments, values may be communicated from sensors, valves, regulators, and/or detectors, to the therapeutic gas delivery system controller, where the value may be communicated as an analog or digital signal over a communication path that may be wired or wireless. In various embodiments, a value may be electrically communicated as an analog current and/or voltage, or as a digital sequence that is representative of the value, where the therapeutic gas delivery system controller may be configured to receive, interpret, and/or store the value, for example with A-to-D converters, buffers, direct memory access (DMA), as well as other hardware, software, and/or firmware that is known in the art.

In exemplary embodiments, an algorithm may determine the run-time-to empty (RTE) using gas pressure information, therapeutic gas source volume information, temperature information, and equations. RTE may be calculated by a therapeutic gas delivery system controller with information generated from using (i) Delivery NO flow sensors, (ii) Redundant (monitoring) flow sensors, (iii) Commands/Settings to NO control valve, (iv) Set dose+Injector module (IM) flow sensor reading (delivery or redundant monitoring flow sensor); and/or (v) Gas source contents pressure sensing.

In exemplary embodiments, RTE may account for (i) Purging (current and future) using therapeutic gas; (ii) System level leaks determined by high pressure or lower (32) pressure decay test; (iii) Residual pressure intended to be left in gas source (gas source not emptied completely); (iv) Concurrent delivery—secondary and Primary running at same time; (v) temperature (e.g., temperature changes may result in changes in pressure, etc.); (vii) filtering (e.g., undesired oscillating values may be filtered, RTE displayed may filter out oscillations, (vi) RTE life extension may immediately update upon changes in set dose; and/or (vii) Improved RTE accuracy with ambient temperature correction.

In one or more embodiments, run-time-to empty information and/or alarms may be provided to users of the therapeutic gas delivery system for one or more of the therapeutic gas sources. In various embodiments, run-time-to empty information and/or alarms may be displayed on a display screen affiliated with one of the one or more gas supply subsystems. In various embodiments, a separate display screen may be affiliated with each of the two or more gas supply subsystems, where each of the displays may be configured to present run-time-to empty information and/or alarms to a user.

Principles and embodiments of the present invention also generally relate to a therapeutic gas delivery system comprising automatic back-up systems that provide simple and easy to use therapeutic gas delivery in the event of failure of a primary gas delivery system, where a back-up system for manual ventilation (e.g., bagging, external manual ventilation device, assisted breathing apparatus, etc.) is sufficiently automated and simple to be utilized by personnel that are otherwise untrained on therapeutic gas delivery systems. In one or more embodiments, a therapeutic gas blending system is configured to provide a controlled gas flow rate to an external manual ventilation device (e.g., bag valve mask) for providing the same set dose to allow a patient to remain ventilated without discontinuation of inhalation therapy.

Delivery and Sampling System Overview

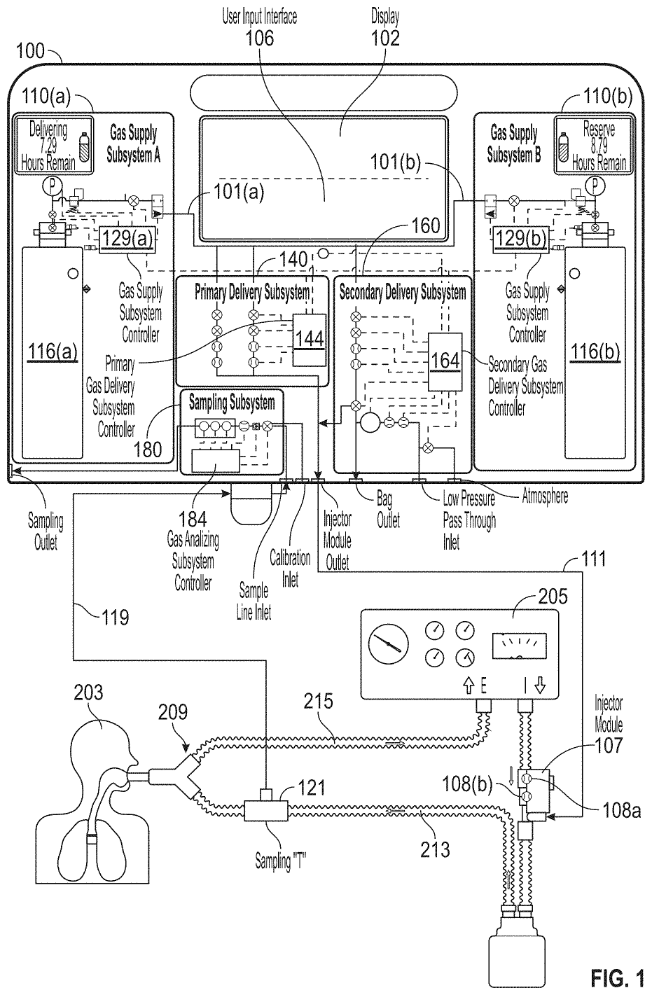

Referring to FIGS. 1-3, an exemplary system for delivering inhaled therapeutic nitric oxide gas (NO) to a patient is illustratively depicted. It will be understood that systems and methods of the present invention may use, modify, and/or be affiliated with any applicable system for delivering therapeutic gas to a patient. For example, systems and methods of the present invention may use, modify, and/or be affiliated with the delivery systems and/or other teachings of U.S. Pat. No. 5,558,083 entitled “NO Delivery System” and/or U.S. Pat. No. 5,752,504 entitled “System for Monitoring Therapy During Calibration”, the contents of both of which are incorporated herein by reference in their entireties.

Systems and methods are, at times, described as being directed towards inhaled nitric oxide (NO). This is merely for ease and is in no way meant to be a limitation. Of course the teachings disclosed herein may, when appropriate, be used for other therapeutic gas, such as, but not limited to, carbon monoxide (CO), hydrogen sulfide (H₂S), etc. Further, therapeutic gas may be supplied from one or more therapeutic gas sources that may be any source of therapeutic gas such as a therapeutic gas contained in a cylinder (e.g., a cylinder containing NO, H₂S), NO gas generator, or the like. Of course other sources of therapeutic gas may be used. For ease, at times, the therapeutic gas source is described as a cylinder, NO cylinder, and the like. This is merely for ease and is in no way meant to be a limitation.

In exemplary embodiments, a therapeutic gas delivery system 100 may be used to deliver therapeutic gas, such as NO, to a patient 203 who may be using an assisted breathing apparatus such as a ventilator 205 or other device used to introduce therapeutic gas to the patient, for example, a nasal cannula, endotracheal tube, face mask, bag valve mask, or the like. For ease, systems and methods of the present invention are described, at times, as being for use with a ventilator. This is merely for ease and is in no way meant to be a limitation. For example, for at least a ventilated patient 203, ventilator 205 may deliver breathing gas to patient 203 via inspiratory limb 213 of patient breathing circuit 209, while patient expiration may flow via an expiratory limb 215 of patient breathing circuit 209, at times, to ventilator 205. Of course other ventilator types are envisioned. For example, a single limb ventilator type system is envisioned that may have a combined inspiratory and expiratory limb.

In exemplary embodiments, systems and methods of the present invention may be used to wild stream blend therapeutic gas with inspiratory flow (e.g., provided from a ventilator, provided from an air and/or oxygen source, etc.). By way of example, described below in more detail, wild stream blending may be accomplished with an injector module 107 coupled to inspiratory limb 213 of breathing circuit 209 enabling NO to be delivered from therapeutic gas delivery system 100 and/or any subsystem (e.g., primary gas delivery subsystem, secondary gas delivery system, etc.) to injector module 107, via delivery conduit 111. This NO may then be delivered, via injector module 107, into inspiratory limb 213 of patient breathing circuit 209 affiliated with ventilator 205 being used to deliver breathing gas to a patient 203. By way of another example, described below in more detail, wild stream blending may be accomplished by blending NO with air and/or oxygen provided from a wall outlet (e.g., high pressure air and/or oxygen that may be provided from a wall outlet in a hospital or cylinder supply, low pressure air and/or oxygen that may be provided from a regulator that may receive air and/or oxygen from a wall outlet in a hospital, gas compressor outlet, etc.). In at least some instances, wild stream blending (e.g., NO with air and/or oxygen provided from a wall outlet) may occur within system 100. In exemplary embodiments, wild stream blending may occur internally within system 100 and/or external of system 100, for example, at injector module 107.

As used herein, “wild stream blended proportional”, “wild stream blending”, “ratio metric blending”, and the like, relates to stream blending, where the main flow stream is an uncontrolled (unregulated) stream that is referred to as the wild stream, and the component being introduced into the wild stream is controlled as a proportion of the main stream, which may typically be blended upstream (or alternatively downstream) of the main stream flowmeter. In various embodiments, the inspiratory flow may be the “wild stream” as the flow (e.g., from the ventilator) is not specifically regulated or controlled by the therapeutic gas delivery system, and the nitric oxide is the blend component, for example, that may be delivered as a proportion of the inspiratory flow through the delivery line and/or conduit 111.

In exemplary embodiments, to at least wild stream blend NO, injector module 107 may be affiliated with at least one flow sensor capable of measuring the mass and/or volume flow rate(s) of at least patient breathing gas in the inspiratory line of the patient breathing circuit. For example, injector module 107 may include one or more breathing circuit gas (BCG) flow sensors 108(a) and/or 108(b) that may measure and communicate to the NO delivery system and/or any subsystem (e.g. primary delivery subsystem, secondary delivery subsystem, etc.) the mass and/or volume flow rate(s) of at least patient breathing gas in the inspiratory line of the breathing circuit passing through injector module 107, and in turn to patient 203. BCG flow sensors may be bi-directional. BCG sensors may also operate via differential pressure measurements. Although shown as being at injector module 107, BCG flow sensors 108(a) and/or 108(b) may be placed elsewhere in the inspiratory limb 213, such as upstream of the injector module 107. Also, instead of receiving flow information from BCG flow sensors 108(a) and/or 108(b), the delivery system may receive flow information directly from the source of inspiratory flow (e.g., ventilator 205, high pressure air and/or oxygen that may be provided from a wall outlet in a hospital, low pressure air and/or oxygen that may be provided from a regulator that may receive air and/or oxygen from a wall outlet in a hospital, etc.) indicative of the flow of breathing gas from the source of inspiratory flow (e.g., ventilator 205, high pressure air and/or oxygen that may be provided from a wall outlet in a hospital, low pressure air and/or oxygen that may be provided from a regulator that may receive air and/or oxygen from a wall outlet in a hospital, etc.).

Therapeutic gas delivery system 100 may include, amongst other things, a first gas supply subsystem 110(a), a second gas supply subsystem 110(b), a primary gas delivery subsystem 140, a secondary gas delivery subsystem 160, and/or a gas analyzing subsystem 180. Therapeutic gas delivery system 100 may also include user interfaces such as display(s) 102 and/or user input interface(s) 106. Further, first gas supply subsystem 110(a) may have user interfaces such as display 112(a) and/or second gas supply subsystem 110(b) may have user interfaces such as display 112(b). Any of the user interfaces may include, but is not limited to buttons, keyboards, knobs, and/or touchscreens, to name a few and/or user input interfaces and/or displays may be combined such that information may be input by users and/or communicated to users. By way of example, central display 102, user input interface 106 and/or displays 112(a), 112(b) may receive and/or provide information indicative of desired settings from the user, such as, but not limited to, the patient's prescription (in mg/kg ideal body weight, mg/kg/hr, mg/kg/breath, mL/breath, gas source concentration, delivery concentration or set dose, duration, etc.), the patient's age, height, sex, weight, etc. User central display 102, user input interface 106 and/or display 112(a), 112(b) may be configured in at least some instances be used to confirm the desired patient dosing (e.g., user input desired dose of NO PPM) using a gas analyzing subsystem 180, as described in greater detail below. In various embodiments, the therapeutic gas delivery system 100 may be in communication with the medical facility's (e.g., hospital) patient information system, where the patient's information and/or prescription may be directly communicated from the patient information system to the therapeutic gas delivery system 100.

It will be understood that any of the elements of system 100 may be combined and/or further separated. For ease elements are, at times, described as being specific to subsystems. This is merely for ease and is in no way meant to be a limitation. Further, information communication paths are, at times, illustrated as dashed lines and/or fluid communication conduits are, at times, illustrated as solid lines. This is merely for ease and is in no way meant to be a limitation.

To at least deliver desired set doses of therapeutic gas to a patient, sample therapeutic gas being delivered to a patient, and/or perform other methods and operations, therapeutic gas delivery system 100 may include a system controller (not shown) and/or subsystems may include subsystem controllers such as, but not limited to, gas supply subsystem controller 129(a), gas supply subsystem controller 129(b), primary gas delivery subsystem controller 144, a secondary gas delivery subsystem controller 164, and/or a gas analyzing subsystem(s) controller 184. The system controller and/or any of the subsystem controllers may comprise one or more processors (e.g., CPUs) and memory, where the system controller and/or any of the subsystem controllers may comprise, for example, a computer system, a single board computer, one or more application-specific integrated circuits (ASICs), or a combination thereof. Processors may be coupled to memory and may be one or more of readily available memory such as random access memory (RAM), read only memory (ROM), flash memory, compact/optical disc storage, hard disk, or any other form of local or remote digital storage. Support circuits may be coupled to processors, to support processors, sensors, valves, analyzing systems, delivery systems, user inputs, displays, injector modules, breathing apparatus, etc. in a conventional manner. These circuits may include cache memory, power supplies, clock circuits, input/output circuitry, analog-to-digital and/or digital-to-analog convertors, subsystems, power controllers, signal conditioners, and the like. Processors and/or memory may be in communication with sensors, valves, analyzing systems, delivery systems, user inputs, displays, injector modules, breathing apparatuses, etc. Communication to and from the system controller may be over a communication path, where the communication path may be wired or wireless, and wherein suitable hardware, firmware, and/or software may be configured to interconnect components and/or provide electrical communications over the communication path(s).

In various embodiments, primary gas delivery subsystem controller 144 and secondary gas delivery subsystem controller 164 may be redundant controllers with duplicate hardware, software and/or firmware (e.g., architected to function with redundancies, etc.), where each subsystem controller may perform the operations of the other subsystem controller and take over in the event of a failure. In various embodiments, therapeutic gas delivery system controller comprises primary gas delivery subsystem controller 144 and secondary gas delivery subsystem controller 164, where primary gas delivery subsystem controller 144 and/or secondary gas delivery subsystem controller 164 may be master controllers and gas supply subsystem controller 129(a) and/or gas supply subsystem controller 129(b) may be slave controllers. In various embodiments, gas analyzer subsystem controller 184 may be a slave controller under primary gas delivery subsystem controller 144 and/or secondary gas delivery subsystem controller 164. Of course, other master slave configurations are and/or other controller configurations are envisioned.

In various embodiments, the therapeutic gas delivery system controller may comprise, but is not limited to, at least one of four subsystem controllers 144, 164, 129(a), and/or 129(b). In exemplary embodiments, each subsystem controller for each subsystem is in electrical communication with components of that subsystem, components of other subsystems and/or any other components affiliated with system 100.

For example, subsystem controller 129(a) may be in electrical communication with the components of a first gas supply subsystem 110(a) (e.g., received therapeutic gas source 116(a), therapeutic gas source valve 117(a), gas source connection valve 118(a), gas pressure sensor 120(a), pressure regulator 122(a), purge valve 124(a), shut off 126(a), gas source identifier 128(a), temperature sensor 130(a), gas source identifier reader 131(a), and/or gas source detector 132(a), etc.), and/or components of another subsystem such as second gas supply subsystem 110(b) (e.g., received therapeutic gas source 116(b), therapeutic gas source valve 117(b), gas source connection valve 118(b), gas pressure sensor 120(b), pressure regulator 122(b), purge valve 124(b), shut off 126(b), gas source identifier 128(b), temperature sensor 130(b), gas source identifier reader 131(b), and/or gas source detector 132(b), etc.), primary gas delivery subsystem 140 (e.g., first primary shut off valve 142(a), first primary high flow control valve 143(a), first primary delivery flow sensor 146(a), first primary confirmatory flow sensor 148(a), second primary shut off valve 142(b), second primary high flow control valve 143(b), second primary delivery flow sensor 146(b), and/or second primary confirmatory flow sensor 148(b), etc.), secondary gas delivery subsystem 160 (e.g., secondary shut off valve 162, secondary medium flow control valve 163, secondary delivery flow sensor 166, and/or a secondary confirmatory flow sensor 168, flow regulating valve 170, low pressure oxygen/air received flow sensor 174, low pressure oxygen/air received confirmatory flow sensor 176, low pressure oxygen/air received pressure sensor 178, and/or overpressure valve 179, etc.), gas analyzing subsystem(s) 180 (e.g., gas sensor 182, gas sensor 186, gas sensor 188, sample gas flow sensor 190; sample pump 192; and/or sample system valve(s) 194, etc.), and/or any other components affiliated with system 100 (e.g., injector module delivery flow sensor 108(a), injector module confirmatory flow sensor 108(b)).

For another example, subsystem controllers 129(b) is in electrical communication with the components of a second gas supply subsystem 110(b) (e.g., received therapeutic gas source 116(b), therapeutic gas source valve 117(b), gas source connection valve 118(b), gas pressure sensor 120(b), pressure regulator 122(b), purge valve 124(b), shut off 126(b), gas source identifier 128(b), temperature sensor 130(b), gas source identifier reader 131(b), and/or gas source detector 132(b), etc.), and/or components of another subsystem such as second gas supply subsystem 110(a) (e.g., received therapeutic gas source 116(a), therapeutic gas source valve 117(a), gas source connection valve 118(a), gas pressure sensor 120(a), pressure regulator 122(a), purge valve 124(a), shut off 126(a), gas source identifier 128(a), temperature sensor 130(a), gas source identifier reader 131(a), and/or gas source detector 132(a), etc.), primary gas delivery subsystem 140 (e.g., first primary shut off valve 142(a), first primary high flow control valve 143(a), first primary delivery flow sensor 146(a), first primary confirmatory flow sensor 148(a), second primary shut off valve 142(b), second primary high flow control valve 143(b), second primary delivery flow sensor 146(b), and/or second primary confirmatory flow sensor 148(b), etc.), secondary gas delivery subsystem 160 (e.g., secondary shut off valve 162, secondary medium flow control valve 163, secondary delivery flow sensor 166, and/or a secondary confirmatory flow sensor 168, flow regulating valve 170, low pressure oxygen/air received flow sensor 174, low pressure oxygen/air received confirmatory flow sensor 176, low pressure oxygen/air received pressure sensor 178, and/or overpressure valve 179, etc.), gas analyzing subsystem(s) 180 (e.g., gas sensor 182, gas sensor 186, gas sensor 188, sample gas flow sensor 190; sample pump 192; and/or sample system valve(s) 194, etc.), and/or any other components affiliated with system 100 (e.g., injector module delivery flow sensor 108(a), injector module confirmatory flow sensor 108(b)). In some examples, the flow regulating valve 170 may be a one-way valve (e.g., directional valve) or a 3 way valve.

For another example, subsystem controllers 144 is in electrical communication with the components of primary gas delivery subsystem 140 (e.g., first primary shut off valve 142(a), first primary high flow control valve 143(a), first primary delivery flow sensor 146(a), first primary confirmatory flow sensor 148(a), second primary shut off valve 142(b), second primary high flow control valve 143(b), second primary delivery flow sensor 146(b), and/or second primary confirmatory flow sensor 148(b), etc.) and/or components of another subsystem such as first gas supply subsystem 110(a) (e.g., received therapeutic gas source 116(a), therapeutic gas source valve 117(a), gas source connection valve 118(a), gas pressure sensor 120(a), pressure regulator 122(a), purge valve 124(a), shut off 126(a), gas source identifier 128(a), temperature sensor 130(a), gas source identifier reader 131(a), and/or gas source detector 132(a), etc.), second gas supply subsystem 110(b) (e.g., received therapeutic gas source 116(b), therapeutic gas source valve 117(b), gas source connection valve 118(b), gas pressure sensor 120(b), pressure regulator 122(b), purge valve 124(b), shut off 126(b), gas source identifier 128(b), temperature sensor 130(b), gas source identifier reader 131(b), and/or gas source detector 132(b), etc.), primary gas delivery subsystem 140 (e.g., first primary shut off valve 142(a), first primary high flow control valve 143(a), first primary delivery flow sensor 146(a), first primary confirmatory flow sensor 148(a), second primary shut off valve 142(b), second primary high flow control valve 143(b), second primary delivery flow sensor 146(b), and/or second primary confirmatory flow sensor 148(b), etc.), secondary gas delivery subsystem 160 (e.g., secondary shut off valve 162, secondary medium flow control valve 163, secondary delivery flow sensor 166, and/or a secondary confirmatory flow sensor 168, flow regulating valve 170, low pressure oxygen/air received flow sensor 174, low pressure oxygen/air outlet confirmatory flow sensor 176, low pressure oxygen/air received pressure sensor 178, and/or overpressure valve 179, etc.), gas analyzing subsystem(s) 180 (e.g., gas sensor 182, gas sensor 186, gas sensor 188, sample gas flow sensor 190; sample pump 192; and/or sample system valve(s) 194, etc.), and/or any other components affiliated with system 100 (e.g., delivery flow sensor 108(a), injector module confirmatory flow sensor 108(b)).

For another example, subsystem controller 164 is in electrical communication with the components of secondary gas delivery subsystem 160 (e.g., secondary shut off valve 162, secondary medium flow control valve 163, secondary delivery flow sensor 166, and/or a secondary confirmatory flow sensor 168, flow regulating valve 170, low pressure oxygen/air received flow sensor 174, low pressure oxygen/air received confirmatory flow sensor 176, low pressure oxygen/air received pressure sensor 178, and/or overpressure valve 179, etc.) and/or components of another subsystem such as first gas supply subsystem 110(a) (e.g., received therapeutic gas source 116(a), therapeutic gas source valve 117(a), gas source connection valve 118(a), gas pressure sensor 120(a), pressure regulator 122(a), purge valve 124(a), shut off 126(a), gas source identifier 128(a), temperature sensor 130(a), gas source identifier reader 131(a), and/or gas source detector 132(a), etc.), second gas supply subsystem 110(b) (e.g., received therapeutic gas source 116(b), therapeutic gas source valve 117(b), gas source connection valve 118(b), gas pressure sensor 120(b), pressure regulator 122(b), purge valve 124(b), shut off 126(b), gas source identifier 128(b), temperature sensor 130(b), gas source identifier reader 131(b), and/or gas source detector 132(b), etc.), primary gas delivery subsystem 140 (e.g., first primary shut off valve 142(a), first primary high flow control valve 143(a), first primary delivery flow sensor 146(a), first primary confirmatory flow sensor 148(a), second primary shut off valve 142(b), second primary high flow control valve 143(b), second primary delivery flow sensor 146(b), and/or second primary confirmatory flow sensor 148(b), etc.), gas analyzing subsystem(s) 180 (e.g., gas sensor 182, gas sensor 186, gas sensor 188, sample gas flow sensor 190; sample pump 192; and/or sample system valve(s) 194, etc.), and/or any other components affiliated with system 100 (e.g., injector module delivery flow sensor 108(a), injector module confirmatory flow sensor 108(b)).

For another example, subsystem controller 184 is in electrical communication with the components of gas analyzing subsystem(s) 180 (e.g., gas sensor 182, gas sensor 186, gas sensor 188, sample gas flow sensor 190; sample pump 192; and/or sample system valve(s) 194, etc.), and/or any other components affiliated with system 100 (e.g., injector module delivery flow sensor 108(a), injector module confirmatory flow sensor 108(b)) and/or components of another subsystem such as first gas supply subsystem 110(a) (e.g., received therapeutic gas source 116(a), therapeutic gas source valve 117(a), gas source connection valve 118(a), gas pressure sensor 120(a), pressure regulator 122(a), purge valve 124(a), shut off 126(a), gas source identifier 128(a), temperature sensor 130(a), gas source identifier reader 131(a), and/or gas source detector 132(a), etc.), second gas supply subsystem 110(b) (e.g., received therapeutic gas source 116(b), therapeutic gas source valve 117(b), gas source connection valve 118(b), gas pressure sensor 120(b), pressure regulator 122(b), purge valve 124(b), shut off 126(b), gas source identifier 128(b), temperature sensor 130(b), gas source identifier reader 131(b), and/or gas source detector 132(b), etc.), primary gas delivery subsystem 140 (e.g., first primary shut off valve 142(a), first primary high flow control valve 143(a), first primary delivery flow sensor 146(a), first primary confirmatory flow sensor 148(a), second primary shut off valve 142(b), second primary high flow control valve 143(b), second primary delivery flow sensor 146(b), and/or second primary confirmatory flow sensor 148(b), etc.), secondary gas delivery subsystem 160 (e.g., secondary shut off valve 162, secondary medium flow control valve 163, secondary delivery flow sensor 166, and/or a secondary confirmatory flow sensor 168, flow regulating valve 170, low pressure oxygen/air received flow sensor 174, low pressure oxygen/air received confirmatory flow sensor 176, low pressure oxygen/air received pressure sensor 178, and/or overpressure valve 179, etc.), and/or any other components affiliated with system 100 (e.g., injector module delivery flow sensor 108(a), injector module confirmatory flow sensor 108(b)).

In one or more embodiments, each subsystem controller 129(a), 129(b), 144, 164, 184 communicates with each of the other subsystem controllers 129(a), 129(b), 144, 164, 184 and at least therapeutic gas delivery system controllers 144, 164 are configured to detect faults, errors, and/or failures, including complete subsystem controller failure. In various embodiments, therapeutic gas delivery system controllers 144, 164 are configured to take over operation of another subsystem controller if and when a fault, error, and/or failure is detected.

The clock circuits may be internal to the system controller and/or provide a measure of time relative to an initial start, for example on boot-up. The system may comprise a real-time clock (RTC) that provides actual time, which may be synchronized with a time-keeping source, for example a network. The memory may be configured to receive and store values for calculations and/or comparison to other values, for example from sensor(s), pumps, valves, etc.

In exemplary embodiments, the memory may store a set of machine-executable instructions (or algorithms), when executed by processors, that may cause the therapeutic gas delivery system and/or any of the subsystems (e.g., functioning independently of one another, any of the subsystems functioning in concert, etc.) to perform various methods and operations.

For example, the delivery subsystems 140, 160 may perform methods to deliver a desired set dose of therapeutic gas (e.g., NO concentration, mg/kg/hr, NO PPM, etc.) to a patient in need thereof comprising: receiving and/or determining a desired set dose of therapeutic gas to be delivered to a patient that may be input by a user; measuring flow in the inspiratory limb of a patient breathing circuit; adjusting a flow control valve to change the amount of therapeutic gas flowing; delivering therapeutic gas containing NO to the patient during inspiratory flow; monitoring inspiratory flow or changes in the inspiratory flow; and/or varying the quantity (e.g. volume or mass) of therapeutic gas delivered in a subsequent inspiratory flow.

For another example, the gas analyzing subsystem 180 may perform methods to determine the concentration of target gas (e.g., NO, CO, etc.) being delivered to a patient comprising: actuating a sampling pump and/or opening a gas sampling valve (e.g., three way valve, directional valve, etc.) to obtain a gas sample from the inspiratory limb of a patient breathing circuit, the gas sample being of blended air and therapeutic gas (e.g., NO) being delivered to a patient; exposing the gas sample to gas sensors (e.g., catalytic type electrochemical gas sensors); obtaining information from the sensor indicative of the concentration of target gas (e.g., NO, nitrogen dioxide, oxygen) being delivered to the patient; and/or communicating to the user the concentration of the target gas.

For yet another example, the gas analyzing subsystem 180 may perform method to perform calibrations (e.g., baseline calibrations) of the gas sensor (e.g., catalytic type sensor, electrochemical gas sensor, NO sensor, etc.) comprising: actuating a sampling pump and/or opening a gas sampling valve (e.g., three way valve, directional valve, etc.) to obtain a gas sample of ambient air (e.g., conditioned room air); exposing the gas sample of ambient air to gas sensors (e.g., catalytic electrochemical NO gas sensors); obtaining information from the sensor indicative of concentration of target gas (e.g., NO) in the ambient air (e.g., 0 PPM NO); and/or generating a new calibration line and/or modifying an existing calibration line by, for example, replacing the initial and/or previous information indicative of zero concentration target gas (e.g., 0 PPM NO) with the obtained information indicative of zero PPM target gas and using the slope of the initial and/or previous calibration line (e.g., slope of initial and/or previous calibration line connecting the initial and/or previous zero and span calibration points). The machine-executable instructions may also comprise instructions for any of the other teachings described herein.

In exemplary embodiments, systems and methods of the present invention may include one or more gas supply subsystems (e.g., first gas supply subsystem 110(a), second gas supply subsystem 110(b), etc.) capable of receiving therapeutic gas (e.g., from a therapeutic gas source) and/or providing the therapeutic gas to a primary and/or secondary delivery subsystem.

First, second gas supply subsystem 110(a), 110(b) may include, but is not limited to, a receptacle (not shown) for receiving a therapeutic gas source 116(a), 116(b). When received, a therapeutic gas source valve 117(a), 117(b) of therapeutic gas source 116(a), 116(b) may be actuated enabling therapeutic gas to exit from therapeutic gas source 116(a), 116(b). In exemplary embodiments, first, second gas supply subsystem 110(a), 110(b) may include, but is not limited to, a gas source coupling 115(a), 115(b); a gas source connection valve 118(a), 118(b); a gas pressure sensor 120(a), 120(b); a pressure regulator 122(a), 122(b); a purge valve 124(a), 124(b); and/or a shut off 126(a), 126(b). In at least some instances, a gas source identifier 128(a), 128(b) and/or a temperature sensor 130(a), 130(b) may be affiliated with gas source 116(a), 116(b). Further, in at least some instance, a gas source identifier reader 131(a), 131(b); and/or a gas source detector 132(a), 132(b); may be used to determine whether or not a gas source has been received and/or loaded properly.

By way of example, gas source coupling 115(a), 115(b) may be configured to receive therapeutic gas source 116(a), 116(b), enabling a fluid flow connection with the therapeutic gas source, connection valve 118(a), 118(b) with system 100, wherein connection valve 118(a), 118(b) is configured to have at least an open state and a closed state. Further, gas pressure sensor 120(a), 120(b) may be adjacent to and in fluid communication with connection valve 118(a), 118(b), wherein connection valve 118(a), 118(b) provides a gas flow path 119(a), 119(b) from the connection valve 118(a), 118(b) to gas pressure regulator 122(a), 122(b). Following this configuration, the gas pressure sensor may be configured to measure a gas pressure at the gas source (e.g., between connection valve 118(a), 118(b) and therapeutic gas pressure regulator 122(a), 122(b), for example, at least when the connection valve 118(a), 118(b) and therapeutic gas source valve 117(a), 117(b) is in an open state, etc.). Further, the gas pressure sensor may be configured to measure gas pressure at therapeutic gas pressure regulator 122(a), 122(b) downstream from gas pressure sensor 120(a), 120(b), connection valve 118(a), 118(b). As used herein, “adjacent to” means abutting or adjoining a neighboring component, where an adjacent downstream component immediately follows the upstream component without other intervening components, and with minimal internal volume (e.g., dead space) between the upstream component and the downstream component. For example, a connection valve and/or gas pressure sensor may have short conduits leading to and from the actual mechanisms, so that even if an inlet of a gas pressure sensor were connected directly to an outlet of the connection valve there may still be a length of fluid flow path between the connection valve mechanism and the gas pressure sensor mechanism. Similarly, the fluid flow path may comprise a short length of conduit 119(a), 119(b) (e.g., tubing, channels, etc.) to which the connection valve 118(a), 118(b) and the gas pressure sensor 120(a), 120(b) may be coupled due to the type of unions used on the gas source valve and the gas pressure sensor. Further, in exemplary embodiments, all conduits placing any and/or all components in fluid connection with therapeutic gas may be minimized and/or eliminated such that “dead ends” (e.g., dead space between the component and the conduit) may be minimized and/or eliminated, for example, as these “dead ends” may be substantially difficult to purge and/or may cause NO₂ generation and/or NO₂ may be substantially difficult to purge from “dead ends”.

While the first gas supply subsystem 110(a) is depicted as being located on the left side of the drawing and the second gas supply subsystem 110(b) is depicted as being located on the right side of the drawing, this is for illustrative purposes of an exemplary embodiment, and should not be construed as a limitation, for which reference should be made to the claims. In addition, while the gas supply subsystems may be referred to as a first gas supply subsystem 110(a) and a second gas supply subsystem 110(b), this is not intended to connote sequence or preference, but is for ease of reference and should not be construed as a limitation, for which reference should be made to the claims. Further, while the gas supply subsystems may be referred to as a first and second gas supply subsystems, this should not be construed that there may only be two gas supply subsystems as additional gas supply subsystems are envisioned, rather it is for ease of reference and should not be construed as a limitation, for which reference should be made to the claims.

In various embodiments, the therapeutic gas source 116(a), 116(b) may be a compressed gas cylinder with an initial gas pressure of about 2000 psi to about 5000 psi having an NO concentration of about 2000 ppm to about 10000 ppm, an initial gas pressure of about 3000 psi having an NO concentration of about 4880 ppm, an initial gas pressure of about 2000 psi to about 5000 psi having an NO concentration of about 400 ppm to about 1600 ppm, and/or an initial gas pressure of about 1800 psi having an NO concentration of about 800 ppm. Of course other initial pressures and/or NO concentrations are envisioned. In one or more embodiments, therapeutic gas source 116(a), 116(b) may be a mini cylinder that may contain a pressurized therapeutic gas at a pressure in the range of about 2000 psi to about 300 psi, or at a pressure of about 3000 psi, where the mini cylinder weighs less than ⅓ the weight of a standard sized gas cylinder (e.g., about 30 lbs. to 50 lbs.) and/or the mini cylinder weights about 1.4 lbs. while providing the same or greater run-time-to-empty compared to previous cylinders (e.g., standard sized gas cylinders). In various embodiments, the lighter mini cylinder(s) enables easier manual cylinder distribution because it require less strength from a user to move and manipulate, and provides more efficient storage in a manner that takes up less physical space than larger standard cylinders. In various embodiments, the mini cylinder may contain a therapeutic gas having a concentration in the range of about 2000 ppm to about 10,000 ppm, or about 4000 ppm to about 10,000 ppm. In various embodiments, the therapeutic gas source is an NO mini cylinder having an NO concentration of about 2000 ppm to about 10000 ppm, and an initial gas pressure of about 3000 psi, or an NO concentration of about 4880 ppm and an initial gas pressure of about 3000 psi.

In exemplary embodiments, the gas supply system receptacle and therapeutic gas source may be configured such that only the desired therapeutic gas source 116(a), 116(b) may be coupled to the gas supply subsystem 110(a), 110(b). In at least some instances, to ensure that the desired gas source is being received, gas source coupling 115(a), 115(b) may be configured to mate with compatible coupling member 114(a), 114(b) of therapeutic gas source 116(a), 116(b). For example, systems and methods of the present invention may include and/or be modified such that they may work with any of the teaching in U.S. Pat. No. 8,757,148 entitled “Devices And Methods For Engaging Indexed Valve And Pressurized Canister Assembly With Collar And For Linear Actuation By Plunger Assembly Into Fluid Communication With Device For Regulating Drug Delivery”, the contents of which is incorporated by reference herein in its entirety. In one or more embodiments, the gas source coupling 115(a), 115(b) and/or matching coupling member 114(a), 114(b) comprises an indexed drug delivery device as described in U.S. Pat. No. 8,757,148. Systems and methods of the present invention may include and/or be modified such that they may work with any of the teaching in U.S. Pat. No. 8,757,148. In various embodiments, the gas source coupling 115(a), 115(b) and matching coupling member 114(a), 114(b) are polarized so a therapeutic gas source 116(a), 116(b) may only be coupled with the gas source coupling with a predetermined orientation. In various embodiments, the therapeutic gas source may be aligned by the gas source coupling, so a gas source identifier 128(a), 128(b) attached to the therapeutic gas source faces in a particular direction. In at least some instances, mechanical and visual guides may be used to aid in the loading of the therapeutic gas source into the receptacle.

In various embodiments, gas supply subsystem 110(a), 110(b) may comprise gas source identifier reader 131(a), 131(b) and/or temperature sensor 130(a), 130(b) that may be positioned within a bay and/or receptacle for receiving therapeutic gas source 116(a), 116(b). Gas source identifier reader 131(a), 131(b) and/or temperature sensor 130(a), 130(b) may be used to, amongst other things, obtain data from the gas source identifier 128(a), 128(b), and/or temperature values of the therapeutic gas source 116(a), 116(b). Further, in at least some instance, a gas source detector 132(a), 132(b) may be used to determine whether or not a therapeutic gas source 116(a), 116(b) has been received and/or mated properly.

In one or more embodiments, therapeutic gas source detector 132(a), 132(b) is operatively associated with the gas source coupling 115(a), 115(b), where the therapeutic gas source detector 132(a), 132(b) is configured to detect when the therapeutic gas source 116(a), 116(b) is properly received by the respective gas source coupling 115(a), 115(b). In various embodiments, the therapeutic gas source detector 132(a), 132(b) is configured to communicate a signal indicating the presence of the therapeutic gas source 116(a), 116(b) to the therapeutic gas delivery system controller and/or respective subsystem controllers 129(a), 129(b). In various embodiments, the therapeutic gas source detector 132(a), 132(b) may be for example a micro-switch, a limit switch, or a proximity detector (e.g., Hall Effect Sensor).

In exemplary embodiments, connection valve 118(a), 118(b) may also prevent loud noise or bang from rapid venting of high pressure gas from conduit/manifold/sample line 119 when removing therapeutic gas source 116(a), 116(b). Connection valve 118(a), 118(b), in at least some instances, may also function to keep air out of the high pressure manifold upstream of pressure regulator 122(a), 122(b).

In exemplary embodiments, pressure regulator 122(a), 122(b) may be configured to reduce the high pressure therapeutic gas from the therapeutic gas source (e.g., 2000 psi, 3000 psi, etc.) to an operating pressure (e.g., 20 psi, 30 psi, etc.).

In exemplary embodiments, primary gas delivery subsystem 140 may be in fluid communication with first gas supply subsystem 110(a) and/or second gas supply subsystem 110(b) such that NO may be received from either and/or both gas supply subsystems (e.g., via conduit 101(a), via conduit 101(b), etc.). Primary gas delivery subsystem 140 may be in fluid communication with a delivery gas pressure sensor(s) 109 (e.g., which may be shared between the primary and secondary delivery sub systems as shown) enabling pressure measurement of NO being supplied from either and/or both gas supply subsystems and/or the therapeutic gas pressure in conduit 101(a) and conduit 101(b). Further, NO received from either and/or both gas supply subsystems may be in fluid communication with a first primary flow control channel 141(a) (e.g., a high flow control channel) and/or a second primary flow control channel 141(b) (e.g., a low flow control channel) such that flow of NO may be controlled. First flow control channel 141(a) may be in fluid communication with a first primary shut off valve 142(a), a first primary high flow control valve 143(a), a first primary delivery flow sensor 146(a), and/or a first primary confirmatory flow sensor 148(a). Similarly, second flow control channel 141(b) may be in fluid communication with a second primary shut off valve 142(b), a second primary high flow control valve 143(b), a second primary delivery flow sensor 146(b), and/or a second primary confirmatory flow sensor 148(b).

In exemplary embodiments, gas delivery subsystem 140 may deliver therapeutic gas, at a desired set dose (e.g., a desired concentration) to a patient (e.g., via an injector module coupled to a patient breathing circuit affiliated with a ventilator). For example, gas delivery subsystem 140 may wild stream blend therapeutic gas (e.g., NO, etc.), via injector module 107, into patient breathing gas in breathing circuit 209, affiliated with ventilator 205, as a proportion of the patient breathing gas. To at least wild stream blend therapeutic gas (e.g. NO, etc.) into patient breathing gas, gas delivery subsystem 140 may receive NO from NO gas source 116(a) and/or NO gas source 116(b), via flow control channel 141(a) and/or flow control channel 141(b), and provide the therapeutic gas, via a delivery conduit 111 that may also be in fluid communication with an injector module 107, which in turn may also be in fluid communication with the inspiratory limb of breathing circuit 209 affiliated with ventilator 205. In various embodiments, therapeutic gas flowing through delivery conduit 111 may be the sum of therapeutic gas flowing through flow control channel 141(a) (e.g., a high flow control channel) and flow control channel 141(b) (e.g., a low flow control channel). Further, to at least wild stream blend therapeutic gas into patient breathing gas, breathing circuit gas flow information may be provided by sensors, such as flow sensor 108(a) and/or flow sensor 108(b) affiliated with injector module 107, in fluid communication with the breathing circuit and/or flow information may be received from the ventilator.

To regulate flow of NO through delivery conduit 111 to injector module 107, and in turn to a patient 203 receiving breathing gas from inspiratory limb 213 of patient breathing circuit 209, one or more flow control valves 143(a) and/or 143(b) (e.g., proportional valves, binary valves, etc.) may open enabling NO delivery to patient 203 by flowing NO received from at least one of the gas supply subsystems by the corresponding flow control channel to injector module 107, via delivery conduit 111, and in turn into inspiratory limb 213 of patient breathing circuit 209 and to patient 203. In at least some instances, therapeutic gas delivery system 100 (e.g., NO delivery system) may include one or more therapeutic gas flow sensors 146(a), 146(b), 148(a), and/or 148(b) that may measure the flow of therapeutic gas through flow control valves 143(a) and/or 143(b) and/or delivery conduit 111, in turn enabling measurement of the flow of therapeutic gas to injector module 107, and in turn to patient 203

In exemplary embodiments, therapeutic gas flow (e.g., NO gas flow) may be wild stream blended proportional to the breathing gas (e.g., air) flow to provide a desired set dose concentration of the therapeutic gas (e.g., NO) in the combined breathing gas and therapeutic gas. For example, a user may input a desired set dose and the delivery system may deliver this set dose to patient 203. Further, therapeutic gas delivery system 100 (e.g., NO delivery system) may execute, for example, using machine-executable instructions, a delivered concentration calculation that confirms that the desired concentration of the therapeutic gas (e.g., NO) is in the combined breathing gas and therapeutic gas using the known concentration of therapeutic gas source 116(a), 116(b); the amount of breathing gas flow in the patient circuit using information from BCG flow sensors 108(a) and/or 108(b) and/or from ventilator 205; and the amount of therapeutic gas flow in delivery conduit 111 going to injector module 107 (and in turn to patient 203) using information from therapeutic gas flow sensors 146(a), 146(b), 148(a), and/or 148(b).

With respect to at least the backup, or secondary, gas delivery subsystem, at times referred to as an “eblender,” “eINOblender,” or the like, of the present invention, some found previous backup systems to be difficult and intimidating, and required extensive training with regard to switch over from ventilator delivered therapeutic gas to manually delivered therapeutic gas. In exemplary embodiments, secondary gas delivery subsystem 160 provides a simple and/or automatic backup system for primary gas delivery subsystem 140, as well as a manual ventilation system as a simple and/or automatic backup system for ventilator 205 supplied breathing gas and patient breathing circuit 209. Further, in exemplary embodiments, the present invention provides an automatic backup, or secondary, delivery system (e.g., eblender), links dose settings of the secondary delivery subsystem to the dose set at a primary delivery subsystem so the patient dose remains at the desired set dose, provides monitoring or confirmation of a set dose, provides backup systems which may, if needed, function independently from the rest of the system.

In exemplary embodiments, similar to primary gas delivery subsystem 140, secondary gas delivery subsystem 160 may be in fluid communication with first gas supply subsystem 110(a) and/or second gas supply subsystem 110(b) such that NO may be received from either and/or both gas supply subsystems (e.g., via conduit 101(a), via conduit 101(b), via conduit 101(a) and conduit 101(b), etc.). Again similar to primary gas delivery subsystem 140, secondary gas delivery subsystem 160 may be in fluid communication a delivery gas pressure sensor(s) 109 (e.g., which may be shared between the primary and secondary delivery subsystems as shown) enabling pressure measurement of NO being supplied from either and/or both gas supply subsystems. Further, NO received from either and/or both gas supply subsystems may be in fluid communication with a secondary flow control channel 161(a) (e.g., a medium flow control channel) such that flow of NO may be controlled. Secondary flow control channel 161(a) may be in fluid communication with a secondary shut off valve 162, a secondary medium flow control valve 163, a secondary delivery flow sensor 166, and/or a secondary confirmatory flow sensor 168. Further, secondary flow control channel 161(a) may be in fluid communication with a flow regulating valve 170 that may control whether flow from secondary gas delivery system goes to injector module 107 or to another assisted breathing apparatus (e.g., a bag valve mask, etc.).

In one or more embodiments, secondary gas delivery subsystem 160 also comprises flow regulating valve 170 that may control whether flow from secondary gas delivery subsystem 160 goes to injector module 107 or to another assisted breathing apparatus (e.g., bag valve mask, etc.). In various embodiments, flow regulating valve 170 may be a three-way valve or directional valve that is configured to direct a gas flow stream to an injector module outlet or low pressure outlet port 167 for a bag valve mask. In various embodiments, the flow regulating valve 170 may include one or more proportional control valves, binary valves, directional valves, or a 3-way valve, where the valve(s) may be configured to direct the gas flow.

In exemplary embodiments, similar to primary gas delivery subsystem 140, secondary gas delivery subsystem 160 may deliver therapeutic gas, at a desired set dose (e.g., a desired concentration), to a patient (e.g., via an injector module coupled to a patient breathing circuit affiliated with a ventilator). For example, secondary gas delivery subsystem 160 may wild stream blend therapeutic gas (e.g., NO, etc.), via injector module 107, into patient breathing gas in breathing circuit 209, affiliated with ventilator 205, as a proportion of the patient breathing gas. To at least wild stream blend therapeutic gas (e.g. NO, etc.) into patient breathing gas, secondary gas delivery subsystem 160 may receive NO from a NO gas source 116(a) and/or NO gas source 116(b), via flow control channel 161(a) and flow regulating valve 170, and provide the therapeutic gas, via a delivery conduit 111 that may also be in fluid communication with an injector module 107, which in turn may also be in fluid communication with the inspiratory limb of breathing circuit 209 affiliated with ventilator 205. Further, to at least wild stream blend therapeutic gas into patient breathing gas, breathing circuit gas flow information may be provided by sensors, such as flow sensor 108(a) and/or flow sensor 108(b) affiliated with injector module 107, in fluid communication with the breathing circuit and/or flow information may be received from the ventilator.

To regulate flow of NO through delivery conduit 111 to injector module 107, and in turn to a patient 203 receiving breathing gas from inspiratory limb 213 of patient breathing circuit 209, at least one flow control valve 163 (e.g., proportional valves, binary valves, etc.) may open enabling NO delivery to patient 203 by flowing NO received from at least one of the gas supply subsystems by the corresponding flow control channel to injector module 107, via delivery conduit 111, to injector module 107, and in turn into inspiratory limb 213 of patient breathing circuit 209 and to patient 203. In at least some instances, therapeutic gas delivery system 100 (e.g., NO delivery system) may include one or more therapeutic gas flow sensors 166 and/or 168 that may measure the flow of therapeutic gas through the at least one flow control valve 163 and/or delivery conduit 111, in turn enabling measurement of the flow of therapeutic gas to injector module 107, and in turn to patient 203.

In exemplary embodiments, therapeutic gas flow (e.g., NO gas flow) may be wild stream blended proportional to the breathing gas (e.g., air) flow to provide a desired set dose concentration of the therapeutic gas (e.g., NO) in the combined breathing gas and therapeutic gas. For example, a user may input a desired set dose and the delivery system may deliver this set dose to patient 203. Further, therapeutic gas delivery system 100 (e.g., NO delivery system) may execute, for example, using machine-executable instructions, a delivered concentration calculation that confirms that the desired concentration of the therapeutic gas (e.g., NO) is in the combined breathing gas and therapeutic gas using the known concentration of therapeutic gas source 116(a), 116(b); the amount of breathing gas flow in the patient circuit using information from BCG flow sensors 108(a) and/or 108(b) and/or from ventilator 205; and the amount of therapeutic gas flow in delivery conduit 111 going to injector module 107 (and in turn to patient 203) using information from therapeutic gas flow sensors 166 and/or 168.

In exemplary embodiments, secondary gas delivery subsystem 160 may receive oxygen and/or air (e.g., from the low pressure outlet of an external gas supply such as a wall gas regulator, from a wall outlet, cylinder, etc.) that may be wild stream blended with NO, for example, from gas supply subsystem A and/or gas supply subsystem B as described above, which in turn may be delivered to an assisted breathing apparatus (e.g., a bag valve mask). By way of example, to at least wild stream blend NO with oxygen and/or air (e.g., from the low pressure outlet of a wall gas regulator, from a wall outlet, etc.) NO received from either and/or both gas supply subsystems may be in fluid communication with secondary flow control channel 161(a) (e.g., a medium flow control channel) such that flow of NO may be controlled. Further, a low pressure conduit 172 may receive low pressure oxygen and/or air (e.g., from the low pressure outlet of a wall gas regulator) via low pressure conduit pass through inlet (e.g., coupled to a low pressure delivery conduit from the low pressure outlet of a wall gas regulator), that may be in fluid communication with a filter, and this received low pressure air may be wild stream blended with NO from either and/or both gas supply subsystems, for example at blending junction 169. Low pressure conduit 172 may be in fluid communication with a low pressure oxygen/air received flow sensor 174, a low pressure oxygen/air received confirmatory flow sensor 176, and/or a low pressure oxygen/air received pressure sensor 178. Following the above example, flow regulating valve 170 (e.g., a three way valve, directional valve, etc.) may be actuated such that NO from secondary flow control channel 161(a) flows to blending junction 169 wherein the NO and oxygen and/or air may be wild stream blended, and in turn, this NO and air and/or oxygen may flow to the assisted breathing apparatus (e.g., a bag valve mask, etc.).

For ease, in at least this configuration, the assisted breathing apparatus is, at times, described as a bag valve mask. Of course other assisted breathing apparatus are envisioned such as, but not limited to, a bag valve mask, nasal cannula, face mask, etc. Accordingly, reference to a bag valve mask is merely for ease and is in no way meant to be a limitation.

In exemplary embodiments, secondary gas delivery subsystem 160 may deliver therapeutic gas, at a desired set dose (e.g., a desired concentration), to a patient, via a bag valve mask, by wild stream blending therapeutic gas (e.g., NO, NO from either and/or both gas supply subsystems, etc.) into low pressure oxygen and/or air (e.g., from the low pressure outlet of a wall gas regulator) as a proportion of low pressure oxygen and/or air. Further, to at least wild stream blend therapeutic gas into low pressure oxygen and/or air, flow information may be provided by sensors, such as flow sensor 174, flow sensor 176, and/or pressure sensor 178, in fluid communication low pressure conduit 172. For ease, only a low pressure oxygen and/or air/O₂ is described. This is merely for ease and is in no way meant to be a limitation, for example, as usage of high pressure oxygen and/or air is envisioned. For example, conduits, valves, flow sensors, etc. may be modified for high pressure and/or therapeutic gas delivery system 100 may include and/or function with a pressure regulator (e.g., to decrease the source pressure, etc.). Accordingly, one skilled in the art will appreciate how therapeutic gas delivery system 100 may function with high pressure oxygen and/or air.

As described above with respect to NO delivery to injector module 107, to regulate flow of NO through flow control channel 161(a) to a bag valve mask, and in turn to a patient 203, at least one flow control valve 163 (e.g., proportional valves, binary valves, etc.) may open enabling NO flow to blending junction 169. At blending junction 169 NO and low pressure oxygen and/or air may be wild stream blended and this NO and oxygen and/or air may in turn flow to a bag valve mask. In at least some instances, therapeutic gas delivery system 100 (e.g., NO delivery system) may include one or more therapeutic gas flow sensors 166 and/or 168 that may measure the flow of therapeutic gas through the at least one flow control valve 163 and/or flow control channel 161(a), in turn enabling measurement of the flow of therapeutic gas to blending junction 169.

In exemplary embodiments, therapeutic gas flow (e.g., NO gas flow) can be wild stream blended proportional to the low pressure oxygen and/or air flow to provide a desired set dose concentration of the therapeutic gas (e.g., NO) in the combined low pressure oxygen and/or air and therapeutic gas. For example, a user may input a desired set dose and the secondary gas delivery subsystem 160 may deliver this set dose to patient 203. Further, therapeutic gas delivery system 100 (e.g., NO gas delivery system) may execute, for example, using machine-executable instructions, a delivered concentration calculation that confirms that the desired concentration of the therapeutic gas (e.g., NO) is in the combined low pressure oxygen and/or air and therapeutic gas using the known concentration of therapeutic gas source 116; the amount of low pressure oxygen and/or air using information from flow sensors 174 and/or 176; and the amount of therapeutic gas flow from flow control channel 161 going to blending junction 169 using information from therapeutic gas flow sensors 166 and/or 168.

In exemplary embodiments, overpressure valve 179 may be in fluid communication with low pressure conduit 172 to, for example, ensure that the pressure in low pressure conduit 172 is not above a predetermined threshold. Overpressure valve 179 may be used to ensure that sensors in fluid communication with low pressure conduit 172 and/or low pressure conduit 172 itself is not damaged by being exposed to high pressure gas (e.g., that may be provided from the high pressure outlet of an oxygen and/or air source).

In at least some instances, system 100 may have fewer or additional delivery subsystems (e.g., primary gas delivery subsystem 140, secondary gas delivery subsystem 160, etc.) and/or system 100 and/or delivery subsystems may have fewer or additional flow control channels and associated elements. For ease, only a primary delivery subsystem having two flow control channels and associated elements (e.g., shut off valves, control valves, flow sensors, confirmatory flow sensors, etc.) and a secondary delivery subsystem having a single flow control channels and associated elements (e.g., shut off valves, control valves, flow sensors, confirmatory flow sensors, etc.) are shown. This is merely for ease and is in no way meant to be a limitation. For example, primary delivery subsystem may include any number of flow control channels, such as, a third flow control channel (not shown) that may be in fluid communication with associated elements (e.g., a third primary shut off valve, a third primary flow control valve, a third primary delivery flow sensor, and/or a third primary confirmatory flow sensor, etc.). For another example, secondary delivery subsystem may include any number of flow control channels, such as, a second flow control channel (not shown) that may be in fluid communication with associated elements (e.g., a second secondary shut off valve, a second secondary flow control valve, a second secondary delivery flow sensor, and/or a second secondary confirmatory flow sensor, etc.). For yet another example, system 100 may include a tertiary delivery subsystem (not shown) that may have any number of flow control channels, such as, a first flow control channel that may be in fluid communication with associated elements (e.g., a first tertiary shut off valve, a first tertiary flow control valve, a first tertiary delivery flow sensor, and/or a first tertiary confirmatory flow sensor, etc.), a second flow control channel that may be in fluid communication with associated elements (e.g., a second tertiary shut off valve, a second tertiary flow control valve, a second tertiary delivery flow sensor, and/or a second tertiary confirmatory flow sensor, etc.), and/or a third flow control channel that may be in fluid communication with associated elements (e.g., a third tertiary shut off valve, a third tertiary flow control valve, a third tertiary delivery flow sensor, and/or a third tertiary confirmatory flow sensor, etc.).

In at least some instances, flow control valves may control various ranges of flow (e.g., high flow, low flow, medium flow, etc.) and/or the same range of flows (e.g., one or more high flow valves, one or more low flow valves, one or more medium flow valves, etc.). For ease, flow control valves (e.g., flow control valve 143(a), flow control valve 143(b), flow control valve 163, etc.) are, at times, described as high flow control valves, low flow control valves, medium flow control valves, and the like. This is merely for ease and is in no way meant to be a limitation. Of course other ranges of flow and/or additional flow control valves and/or ranges are envisioned.

In at least some instances, flow control valves (e.g., flow control valve 143(a), flow control valve 143(b), flow control valve 163, etc.) may be any type of valve capable of controlling gas flow such as, but not limited to, proportional valves, binary valves, any combination or further separation thereof, and/or any other type of valve.

In at least some instances, therapeutic gas flow sensors 146(a), 146(b), 148(a), 148(b), 166, and/or 168 and flow control valves (e.g., flow control valve 143(a), flow control valve 143(b), flow control valve 163, etc.) in corresponding flow control channels may be configured such that the flow sensors may be upstream, downstream, and/or combinations thereof of the corresponding flow control valve(s). Therapeutic gas delivery system 100 is described, at times, as having flow sensors one corresponding confirmatory flow sensor. This is merely for ease and is in no way meant to be a limitation because, for example, more than one corresponding confirmatory flow sensor is envisioned.

In one or more embodiments, the therapeutic gas delivery system 100 may have one or more inlet ports and outlet ports, where the ports may be general ports to allow connecting and/or fluid communication of the system to external components (e.g., injector module outlet port), or dedicated ports that provide connection and/or fluid communication of external components to particular subsystem(s) and/or components to provide specific system functions (e.g., low pressure air inlet port, gas analyzing inlet port). In various embodiments, the inlet ports and outlet ports may comprise connectors, for example quick disconnect gas connectors, hose barb connectors, and hose couplings, to name a few. In exemplary embodiments, therapeutic gas delivery system 100 may comprise a primary outlet port (also referred to as an injector module outlet port) for connection to an injector module, a low pressure outlet port 167 for connection to a manual ventilation device, and a low pressure inlet port 165 for connection to a low pressure air/O₂ supply.

In exemplary embodiments, therapeutic gas delivery system 100 may allow a user to input a desired set dose of the therapeutic gas (e.g., NO in PPM) and the therapeutic gas delivery system may confirm that the desired set dose of the therapeutic gas is being delivered to the patient by calculating the delivery concentration (e.g., as described above) as well as using gas analyzing subsystem 180 to confirm the desired set dose of the therapeutic gas (e.g., NO) is being delivered to the patient. Gas analyzing subsystem 180 may include, but is not limited to numerous sensors such as, but not limited to, an electrochemical NO gas sensor 182, which may have a catalytic type electrode material with high catalytic activity for the electrochemical reactions of the sensor, a catalytic type electrochemical nitrogen dioxide gas sensor 186, and a galvanic type electrochemical oxygen gas sensor 188, to name a few; a sample gas flow sensor(s) 190; a sample pump(s) 192; sample system valve(s) 194; and/or controller 184. Sensors 182, 186, and 188 may be in series and/or parallel and/or may be in any order. For ease, sensors 182, 186, and 188 are illustratively depicted as being in series. This is merely for ease and is in no way meant to be a limitation. In various embodiments, the NO sensor may be an electrochemical sensor, which may comprise two electrodes, including a sensing and a counter electrode, separated by a thin layer of electrolyte.

In exemplary embodiments, gas analyzing subsystem 180 may sample and/or measure the concentration of various gases being delivered to a patient. The concentration of NO being delivered to patient 203 may be sampled and exposed to NO sensor 182, which in turn may output information indicative of the concentration of NO in the breathing gas (e.g., NO PPM). For example, a sample of the gas being delivered to the patient may be sampled via a sample line 119 that is in fluid communication with inspiratory limb 213 of breathing circuit 209 affiliated with ventilator 205 (e.g., breathing apparatus). Sample line 119 may be in fluid communication with inspiratory limb 213 via a sampling “T” 121 which may be coupled to inspiratory limb 213. This gas sample from the inspiratory limb, via sample line 119, may flow and/or be pulled to the gas sensors 182, 186, 188 (e.g., NO sensor 182, nitrogen dioxide gas sensor 186, oxygen gas sensor 188, etc.). Flow in sample line 119 may be regulated via valve 194 and/or sample pump 192. Sample line mass or volume flow may be measured using flow sensor 190. Sample line 119 may also be in fluid communication with a gas sample conditioner 196 that may condition the sample gas, for example, by extracting fluids, placing the sample at the appropriate humidity, removing contaminants from the sample, and/or may condition the sample gas in any other way as desired.

In exemplary embodiments, gas analyzing subsystem 180 may perform calibrations (e.g., baseline calibrations, span calibrations, etc.) of the gas sensor (e.g., catalytic type electrochemical gas sensor, etc.) by sampling and/or measuring the concentration of target gases in a controlled sample (e.g., baseline sample, span sample, etc.), where a span sample is a target gas (i.e., nitric oxide) with a specific known and controlled concentration within a range of interest (e.g., 10 PPM, 25 PPM, 50 PPM, 80 PPM, etc.) and/or where a baseline sample is a gas containing zero concentration of a target gas (i.e., conditioned ambient air containing zero nitric oxide). For example, a sample of ambient gas and/or span gas may be sampled via a sample line 119 and/or 198 that may be in fluid communication with valve 194. This gas sample may flow and/or be pulled to the gas sensors (e.g., NO sensor 182, etc.) wherein the flow may be regulated via valve 194 (e.g., a three way valve, a directional valve, etc.) and/or sample pump 192. Sample line flow may be measured using flow sensor 190.

In exemplary embodiments, sample line 119 may also be in fluid communication with a gas sample conditioner (not shown) that may condition the sample gas, for example, by extracting fluids, placing the sample at the appropriate humidity, removing contaminants from the sample, and/or may condition the sample gas in any other way as desired. For example, the ambient air used for the baseline calibration may be scrubbed of any undesirable gases using a scrubber material. By way of example, this scrubbing material may be an inline Potassium permanganate scrubber material capable of scrubbing the ambient air removing NO and NO₂. With the NO and NO₂ removed from the ambient air, the scrubbed air may be used for a zero calibration as these undesirable gases have been removed hence they are at 0 PPM. If needed, a similar technique (e.g., using an inline scrubber material) may be done for span gas.

### Therapeutic Gas Source Management

In exemplary embodiments, at least some aspect of the present invention relate to systems, methods, and/or process for, amongst other things, managing use of one or more therapeutic gas sources, receipt of therapeutic gas source, receiving information from therapeutic gas sources, performing run-time-to-empty calculations, providing information pertaining run-time-to-empty to users, and/or providing alarms, to name a few.

In one or more embodiments, therapeutic gas source 116(a), 116(b) may be received by receptacle/gas supply subsystem 110(a), 110(b). To be received by receptacle/gas supply subsystem 110(a), 110(b), coupling member 114(a), 114(b) of therapeutic gas source 116(a), 116(b) may be required to mate with gas source coupling 115(a), 115(b) of receptacle/gas supply subsystem 110(a), 110(b). After being received, therapeutic gas source 116(a), 116(b) may be actuated (opened) thereby placing therapeutic gas source 116(a), 116(b) in fluid communication with gas pressure sensor 120(a), 120(b), which measures the pressure of the gas in therapeutic gas source 116(a), 116(b).

In exemplary embodiments, when received by therapeutic gas delivery system 100, gas source identifier reader 131(a), 131(b) may read gas source identifier 128(a), 128(b), which has recorded thereon the actual measured concentration of the therapeutic gas in gas source 116(a), 116(b) and/or the manufacturer's target gas concentration for therapeutic gas source 116(a), 116(b). Gas source identifier 128(a), 128(b) may also have recorded thereon additional data such as, but not limited to, the wetted volume of the gas source, the identity of the therapeutic gas, and/or its expiration date, to name a few. Data recorded on gas source identifier 128(a), 128(b) and gas pressure measured by gas pressure sensor 120(a), 120(b) may be communicated to therapeutic gas delivery system controller and stored in memory. In exemplary embodiments, at least some of the information recorded on gas source identifier 128(a), 128(b) may be used for run-time-to-empty calculations.

In one or more embodiments, gas source identifier 128(a), 128(b) may be radio-frequency identification (RFID) tags with read/write (R/W) memory in the communicating component, used to transmit data to the system controller(s) via an RFID reader 131(a), 131(b), bar codes and/or QR codes.

In various embodiments, gas source identifier reader 131(a), 131(b) may be an imaging device (e.g., camera) for reading and communicating actual gas concentration data on a QR code, or a barcode scanner for reading and communicating actual gas concentration data on a barcode. In one or more embodiments, gas source identifier reader 131(a), 131(b) may be a component of the bay or receptacle for the engagement of the therapeutic gas source in the therapeutic gas supply subsystem 110(a), 110(b). The bay or receptacle may further include means for correctly aligning the gas source within the bay or receptacle for reading the actual therapy gas concentration data. Corresponding means for aligning may be incorporated in the therapy gas source via imaging camera, or an RFID reader for reading and communicating actual gas concentration data on an RFID tag, where the tag may be unreadable if facing in the wrong direction. In certain embodiments, the means for aligning may include a keying arrangement between the gas source (e.g., via the gas source valve body) and the bay or receptacle receiver, or markings on the bay or receptacle and the gas source to be aligned upon placement of the gas source into the therapeutic gas delivery system. Such means for gas source alignment may also be used to prevent attachment of an incorrect gas source to the therapy gas delivery system.

In one or more embodiments, shut off valve 126(a), 126(b), which may be located downstream from and in fluid communication with the purge valve 124(a), 124(b), may provide a gas barrier between the gas supply subsystem 110(a), 110(b) and the primary gas delivery subsystem 140 and/or secondary gas delivery subsystem 160, and may block gas flow to therapeutic gas conduit(s) 101(a), 101(b). Shut off valve(s) 126(a), 126(b) may be binary valve(s). In one or more embodiments, a therapeutic gas conduit 101(a), 101(b) may provide a gas flow path (e.g., an enclosed gas flow path, tubing, channel, etc.) at least from the at least one gas supply subsystem to at least one primary gas delivery subsystem (e.g., primary gas delivery subsystem 140, etc.) and/or the at least one secondary gas delivery subsystem (e.g., secondary gas delivery subsystem 160).

In various embodiments, gas conduit pressure sensor 109 is connected to and in fluid communication with therapeutic gas conduit(s) 101(a), 101(b), is configured to measure a gas pressure in therapeutic gas conduit(s) 101(a), 101(b) being delivered to primary gas delivery subsystem 140 and/or secondary gas delivery subsystem 160, and/or is configured to be in communication, via a communication path, with a therapeutic gas delivery system controller. In various embodiments, gas pressure sensor 120(a), 120(b) is on the high pressure side (e.g., 3000 psi) of therapeutic gas pressure regulator 122(a), 122(b), while gas conduit pressure sensor 109 is on the regulated/downstream pressure side (e.g., 30 psi) of therapeutic gas pressure regulator 122(a), 122(b).

In one or more embodiments, with therapeutic gas source 116(a), 116(b) received by system 100, NO may be provided from either and/or both gas supply subsystem 110(a), 110(b) and, in turn, be fluidly communicated with first flow control channel 141(a) (e.g., a high flow control channel) and/or second flow control channel 141(b) (e.g., a low flow control channel) such that flow of the therapeutic gas (e.g., NO) may be controlled. In various embodiments, a high flow control channel may be configured to supply higher flow rates and thereby higher doses more accurately, whereas a low flow control channel may be configured to supply lower flow rates and thereby lower doses more accurately.

In exemplary embodiments, system 100 may automatically activate when dose set and injector module flow (e.g., inspiratory flow, forward flow, etc.) are above a pre-determined threshold, which would be flow rates indicative of an operational ventilator. By way of example, primary gas delivery subsystem 140 and/or secondary gas delivery subsystem 160 may automatically activate when dose set and injector module flow are determined to be above a pre-determined threshold. This may be accomplished, because, as noted above, the therapeutic gas delivery system controller (e.g., primary gas delivery subsystem controller 144 and/or secondary gas delivery subsystem controller 164, etc.) may be configured to communicate with first, second primary shut off valve 142(a), 142(b); first, second primary flow control valve 143(a), 143(b); first, second primary delivery flow sensor 146(a), 146(b); first, second primary confirmatory flow sensor 148(a), 148(b); secondary shut off valve 162, secondary medium flow control valve 163, secondary delivery flow sensor 166, and/or secondary confirmatory flow sensor 168, flow regulating valve 170, injector module delivery flow sensor 108(a), and/or injector module confirmatory flow sensor 108(b). In at least some instances, the first, second primary shut off valve 142(a), 142(b); first, second primary flow control valve 143(a), 143(b); first secondary shut-off valve 162, and first secondary flow control valve 163 are normally closed.

In various embodiments, primary delivery subsystem controller 144 may compare flow rate values received from first primary delivery flow sensor 146(a) and first primary confirmatory flow sensor 148(a) for the therapeutic gas, and may provide an alarm, recommend replacing at least one of the sensors, perform verification processes (described below in greater detail) to confirm which sensor is not functioning properly, and/or provide flow information from the functioning flow sensor, etc. if therapeutic gas flow rates measured at first primary delivery flow sensor 146(a) and first primary confirmatory flow sensor 148(a) differ from each other by greater than a threshold amount of about 10%, or about 7%, or about 5%, or about 2.5%, or about 2%, or about 1%, or about 0.5%.

In various embodiments, primary delivery subsystem controller 144 may compare flow rate values received from second primary delivery flow sensor 146(b) and second primary confirmatory flow sensor 148(b) for the therapeutic gas, and may provide an alarm, recommend replacing at least one of the sensors, perform verification processes (described below in greater detail) to confirm which sensor is not functioning properly, and/or provide flow information from the functioning flow sensor, etc. if therapeutic gas flow rates measured at second primary delivery flow sensor 146(b) and second primary confirmatory flow sensor 148(b) differ from each other by greater than a threshold amount of about 10%, or about 7%, or about 5%, or about 2.5%, or about 2%, or about 1%, or about 0.5%.

In exemplary embodiments, the arrangement of first, second primary delivery flow sensor 146(a), 146(b) and/or first, second primary confirmatory flow sensor 148(a), 148(b) provides monitoring of the primary delivery subsystem that may consist of at least 3 sets of sensors for triangulation of failure, including injector module delivery flow sensor 108(a) and/or injector module confirmatory flow sensor 108(b), first, second primary delivery flow sensor 146(a), 146(b) and/or first, second primary confirmatory flow sensor 148(a), 148(b), and therapeutic gas sensor 182, where flow rate values from the flow sensors may be compared, ratio-metric calculations be performed and compared to the therapeutic gas sensor value to determine if any of these components have failed, or need service and/or calibration. Further, in at least some instances, therapeutic gas delivery system 100 may automatically perform verification processes (e.g., triangulation of failure, etc.) during delivery of therapeutic gas to a patient and/or if therapeutic gas sensor identifies a failed sensor, valve, or other component is identified then therapeutic gas delivery system 100 may use information from another sensor, valve, or other component that is functioning. By way of example, during delivery of therapeutic gas to a patient, therapeutic gas delivery system 100 may perform verification processes (e.g., triangulation of failure) and identify that a flow sensor is not functioning and therefor use flow information from a confirmatory flow sensors. Similar calculation and comparisons are described for a pre-use performance verification described herein.

### Secondary Delivery Subsystem

In exemplary embodiments, at least some aspect of the present invention relate to systems, methods, and/or process for, amongst other things, providing therapeutic gas from one or more sources, providing therapeutic gas from a primary delivery subsystem, providing therapeutic gas from a secondary delivery subsystem, providing therapeutic gas from a primary and secondary delivery subsystem, providing therapeutic gas to a ventilated patient, and/or providing therapeutic gas to an assisted breathing apparatus, to name a few.

In exemplary embodiments, as described above, therapeutic gas delivery system 100 may include a plurality of delivery subsystems capable of receiving therapeutic gas from a plurality of sources and deliver the received therapeutic gas to a patient in need thereof using various techniques (e.g., delivery to injector module from primary delivery subsystem, delivery to injector module from secondary delivery subsystem, delivery to injector module from primary delivery subsystem and secondary delivery subsystem, delivery to an external manual ventilation device from secondary delivery subsystem, delivery to an external manual ventilation device from primary delivery subsystem, etc.). To accomplish at least the above, therapeutic gas delivery system 100 may include primary gas delivery subsystem 140, which may comprise two flow control channels and secondary gas delivery subsystem 160, which may comprise a secondary flow control channel, such that a therapeutic gas delivery system 100 comprises three redundant flow control channels in fluid communication with therapeutic gas conduit(s) 101(a), 101(b).

In exemplary embodiments, NO received from either and/or both gas supply subsystems may be in fluid communication with a secondary flow control channel 161(a) (e.g., a medium flow control channel) such that flow of NO may be controlled. Secondary flow control channel 161(a) may be in fluid communication with a secondary shut off valve 162, a secondary medium flow control valve 163, a secondary delivery flow sensor 166, and/or a secondary confirmatory flow sensor 168. Further, secondary flow control channel 161(a) may be in fluid communication with a flow regulating valve 170, which may be a 3-way valve or directional valve that may control whether flow from the secondary gas delivery subsystem 160 goes to injector module 107 or to outlet port 167 to another external manual ventilation device (e.g., bag valve mask). In various embodiments, secondary gas delivery subsystem 160 may have its own purge valve in fluid communication with flow control channel 161(a).

In exemplary embodiments, flow regulating valve 170 may be oriented (e.g., in reverse), so that at least one of the flow controllers in the primary gas delivery subsystem may back up the flow controller in secondary system. In various embodiments, flow regulating valve 170 may switch from being closed or delivering a therapeutic gas to the low pressure outlet port 167 to delivering the therapeutic gas to the primary outlet and therapeutic gas delivery conduit 111 at the same dose as was being delivered by primary gas delivery subsystem 140.

In exemplary embodiments, the secondary delivery subsystem controller, primary delivery subsystem controller, and/or the system controller may detect problems (e.g. loss of communication with primary system) and, in at least some instances, respond to the detected problem. For example, delivery subsystem controller(s) 144 and/or 164 may detect a failure in one or more of the flow control channels of primary gas delivery subsystem 140, automatically switch therapeutic gas flow control to a flow control channel of secondary gas delivery subsystem 160, and switch flow regulating valve 170 to deliver the therapeutic gas to low pressure conduit 172 (e.g., primary outlet) and, in turn, to therapeutic gas delivery conduit 111. For another example, delivery subsystem controller(s) 144 and/or 164 may detect a failure in one of the two flow control channels of primary gas delivery subsystem 140 and automatically switch from the failed therapeutic gas flow control to the other functioning flow control channel of primary gas delivery subsystem 140 and/or change the flow of the functioning flow control channel to provide the desired set dose. Using at least the above technique the patient may be able stay on the ventilator with delivery at the same dose setting. For example, since the therapeutic gas is still delivered to therapeutic gas delivery conduit 111 and injector module 107, the gas analyzing subsystem still detects the amount of therapeutic gas being delivered by secondary gas delivery subsystem 160, and may display the amount to a user to allow continued monitoring of the delivered dose.

In various embodiments, secondary gas delivery subsystem 160 may have its own internal battery backup (not shown) separate from the main system battery (not shown). In various embodiments, two or more batteries may be able to power primary gas delivery subsystem 140 and secondary gas delivery subsystem 160, so in the event of a battery failure the other may be available.

In one or more embodiments, secondary delivery subsystem controller 164 and/or system controller may be configured to perform ratio-metric flow calculations for the concentration of therapeutic gas being delivered to the patient 203 based on the values from secondary delivery flow sensor 166 and/or secondary confirmatory flow sensor 168, and from injector module delivery flow sensor 108(a) and/or injector module confirmatory flow sensor 108(b), which measure ventilation flow rate in the breathing circuit or nasal cannula passing through the injector module. In exemplary embodiments, secondary delivery flow sensor 166 and/or secondary confirmatory flow sensor 168 provides monitoring of the secondary delivery subsystem that may consist of 3 sets of sensors for triangulation of failure, including injector module delivery flow sensor 108(a) and/or injector module confirmatory flow sensor 108(b), secondary delivery flow sensor 166 and/or secondary confirmatory flow sensor 168, and therapeutic gas sensor 182, where flow rate values from the flow sensors may be compared, the ratio-metric calculations done and compared to the therapeutic gas sensor value to determine if any of these components have failed.

In various embodiments, secondary delivery subsystem controller 164 may compare flow rate values received from secondary delivery flow sensor 166 and secondary confirmatory flow sensor 168 for therapeutic gas, and may provide an alarm, recommend replacing at least one of the sensors, perform verification processes (described below in greater detail) to confirm which sensor is not functioning properly, and/or provide flow information from the functioning flow sensor, etc. if the therapeutic gas flow rates measured at the secondary delivery flow sensor 166 and the secondary confirmatory flow sensor 168 differ from each other by greater than a threshold amount of about 10%, or about 7%, or about 5%, or about 2.5%, or about 2%, or about 1%, or about 0.5%.

In one or more embodiments, as described above, secondary gas delivery subsystem 160 also comprises two or more flow sensors 176, 174 along the gas flow path between the flow regulating valve 170 and a low pressure inlet port 165, where the two or more flow sensors 174, 176 are in fluid communication with each other and are located relative to each other in series, parallel, skewed, and/or any other configuration; pressure sensor 178 in fluid communication with the two or more flow sensors 174, 176, and/or low pressure outlet port 167. Further, the gas flow path from the inlet may intersect the gas flow path from the flow regulating valve 170 at blending junction 169. In various embodiments, secondary delivery flow sensor 166, and secondary confirmatory flow sensor 168 are in fluid communication with each other and are located relative to each other in series, parallel, skewed, and/or any other configuration.

In exemplary embodiments, secondary gas delivery subsystem 160 may automatically activate and/or deactivate when air/O₂ flow (e.g., low pressure air/O₂ from a wall outlet, from a compressor, etc.) is above a predetermined threshold and/or below a predetermined threshold. For example, if flow sensor(s) 176, 174 detect air/O₂ flow rates greater than pre-set threshold (e.g. 0.5 SLPM for 2 seconds, flow rates indicative of wall flow, etc.) then secondary flow control valve 163 may automatically activate to deliver the set dose. Further, if flow sensor(s) 176, 174 detect air/O₂ flow rates lower than pre-set threshold (e.g. 0 flow for 2 seconds) then secondary flow control valve 163 may automatically deactivate. Using at least the above, secondary gas delivery subsystem 160 may automatically activate and/or deactivate when a user (e.g., nurse, doctor, etc.) turns on and/or off air/O₂ flow. In at least some instances, therapeutic gas delivery system 100, may alert the user of deactivation of NO delivery, for example, in case Air/O₂ was mistakenly turned off and/or in case the low pressure tubing became disconnected from the secondary delivery system. Further, in exemplary embodiments, when flow is detected a prompt may be provided for the user to squeeze the bag valve mask a multiple times to perform a purge of the bag valve mask.

In exemplary embodiments, secondary gas delivery subsystem 160 may detect when and/or activate in response to squeezing of a valve mask bag. For example, manual activation may prompt the user to start air/O₂ flow at wall flowmeter and may trigger a prompt to squeeze the bag valve mask multiple times to purge NO₂ (e.g., that may be generated as NO delivery may begin automatically in response to air/O₂ flow detection, etc.) During each squeeze of the bag valve mask flow rates may be detected above a pre-set threshold (e.g., change in flow indicative of squeezing the bag valve mask) and secondary flow control valve 163 may automatically activate to deliver the set dose. Similarly, when no squeeze of the bag valve mask is detected (e.g., flow rates below a pre-set threshold indicative of no squeezing of the bag valve mask), then secondary flow control valve 163 may automatically deactivate to halt deliver of the set dose.

In exemplary embodiments, secondary gas delivery subsystem 160 may detect when a user (e.g., nurse, doctor, etc.) attached air/O₂ flow (e.g., low pressure air/O₂ from a wall outlet, from a compressor, etc.) incorrectly, for example, such that the bag valve mask is coupled to the inlet port rather than the outlet port and/or the air/O₂ flow is coupled to the outlet port 167 rather than the inlet port 165 and, in at least some instances, provide an alert. For example, secondary delivery flow sensor 166, secondary confirmatory flow sensor 168, low pressure flow sensor 174, and/or low pressure confirmatory flow sensor 176 (e.g., bi-directional flow sensors) may detect air/O₂ flow in reverse (e.g. hooked up backwards) and may provide an alarm if reverse flow is detected.

In exemplary embodiments, when the dose is set to 0, secondary gas delivery subsystem 160 may still automatically activate upon detection of activation conditions (e.g., such as those described above) and deliver default dose of 20 ppm NO when a system dose may be set to 0. In various embodiments, secondary gas delivery subsystem 160 dose may be set to a different dose than for the primary gas delivery subsystem 140, where a user may input separate doses for the primary gas delivery subsystem 140 and the secondary gas delivery subsystem 160. In various embodiments, secondary gas delivery subsystem 160 may detect elevated humidity or changes in gas density and compensate and/or provide an alarm.

In one or more embodiments, flow sensor 174 may be a low pressure delivery flow sensor and flow sensor 176 may be a low pressure confirmatory flow sensor. In various embodiments, secondary delivery subsystem controller 164 may compare the flow rate values received from low pressure delivery flow sensor 174 and low pressure confirmatory flow sensor 176 for the low pressure breathing gas, and may provide an alarm, recommend replacing at least one of the sensors, perform verification processes (described below in greater detail) to confirm which sensor is not functioning properly, and/or provide flow information from the functioning flow sensor, etc. if the breathing gas flow rates measured at the low pressure confirmatory flow sensor 176, and the low pressure delivery flow sensor 174 differ from each other by greater than a threshold amount of about 10%, or about 7%, or about 5%, or about 2.5%, or about 2%, or about 1%, or about 0.5%.

In exemplary embodiments, secondary gas delivery subsystem 160 may receive oxygen and/or air from a low pressure gas supply (e.g., from the low pressure outlet of a wall gas regulator, from a wall outlet, etc.) that may be wild stream blended with NO, for example, from gas supply subsystem A 110(a) and/or gas supply subsystem B 110(b) as described above, which in turn may be delivered to an assisted breathing apparatus (e.g., bag valve mask). In various embodiments, the low pressure gas supply may be a wall supply and/or a pressurized cylinder configured to provide air, oxygen, or a combination thereof. By way of example, to at least wild stream blend NO with oxygen and/or air (e.g., from the low pressure outlet of a wall gas regulator, from a wall outlet, etc.) NO received from either and/or both gas supply subsystems may be in fluid communication with secondary flow control channel 161(a) (e.g., a medium flow control channel) such that flow of NO may be controlled. Further, low pressure conduit 172 may receive low pressure oxygen and/or air (e.g., from the low pressure outlet of a wall gas regulator) via low pressure conduit pass through inlet port (e.g., coupled to a low pressure delivery conduit from the low pressure outlet of a wall gas regulator) and this received low pressure air may be wild stream blended with NO from either and/or both gas supply subsystems, for example at blending junction 169. Blending junction 169 may be configured to mix a therapeutic gas delivered by flow control channel 161(a) with a gas received at least one of the one or more inlet ports. Low pressure conduit 172 may be in fluid communication with low pressure oxygen/air received flow sensor 174, low pressure oxygen/air received confirmatory flow sensor 176, and/or low pressure oxygen/air received pressure sensor 178. Following the above example, flow regulating valve 170 may be actuated such that NO from secondary flow control channel 161 flows to blending junction 169 wherein the NO and oxygen and/or air may be wild stream blended, and in turn, this NO and air and/or oxygen may flow to the assisted breathing apparatus (e.g., bag valve mask, nasal cannula, etc.). In exemplary embodiments, a pressure relief valve 179 may be in fluid communication with low pressure conduit 172 to, for example, ensure that the pressure in low pressure conduit 172 is not above a predetermined threshold. In various embodiments, secondary delivery subsystem controller 164 may detect when pressure sensor 178 measures a pressure above or below a predetermined range, which may indicate a high pressure gas source has been attached to low pressure inlet port 165, or an assisted breathing apparatus (e.g., bag valve mask) has become disconnected from low pressure outlet port 167. Alarms may be provided when secondary delivery subsystem controller 164 detects that pressure sensor 178 measures a pressure above or below a predetermined range. Measured air/O₂ pass-thru flow rate may be displayed on display 102, 112(a), 112(b). Dosing and delivery info may be displayed on display 102, 112(a), 112(b) along with confirmation of delivery.

In exemplary embodiments, gas analyzing subsystem 180 may detect a failure of NO sensor 182, a nitrogen dioxide gas sensor 186, and/or an oxygen gas sensor 188, where the gas analyzer subsystem controller 184 may detect a failure of NO sensor 182 to ratio-metrically calculated value of NO concentration for one or more flow control channels. If a failure or error is detected at the gas analyzer, then rather than lose monitoring the therapeutic gas delivery system may display the ratio-metric delivered NO concentration from delivery or confirmatory sensors in place of the gas analyzer measured NO concentration and alert the user of the issue.

In at least some instances, gas analyzing subsystem 180 may require calibration before being operatively associated with an inspiratory limb 213 and/or injector module 107 to sample therapeutic gas(es) and/or during delivery of therapeutic gas to a patient to ensure the gas analyzing subsystem 180 is functioning properly. For example, gas analyzing subsystem 180 may perform calibrations (e.g., baseline calibrations, span calibrations, etc.) of the gas sensor (e.g., catalytic type electrochemical gas sensor, etc.) by sampling and/or measuring the concentration of target gases in a controlled sample (e.g., baseline sample, span sample, etc.), where a span sample is a target gas (i.e., nitric oxide) with a specific known and controlled concentration within a range of interest (e.g., 10 PPM, 25 PPM, 50 PPM, 80 PPM, etc.) and/or where a baseline sample is a gas containing zero concentration of a target gas (i.e., conditioned ambient air containing zero nitric oxide). For example, a sample of ambient gas and/or span gas may be sampled via a sample line 119 that may be in fluid communication with valve 194. This gas sample may flow and/or be pulled to the gas sensors (e.g., NO sensor 182, etc.) wherein the flow may be regulated via valve 194 (e.g., a three way valve, a directional valve, etc.) and/or sample pump 192. Sample line flow may be measured using flow sensor 190. A gas sample from ambient gas and/or span gas, via sample line 119, may flow and/or be pulled to the gas sensors (e.g., NO sensor 182). Flow in sample line 119 may be regulated via valve 194 (e.g., a three way valve, a directional valve, etc.) and/or sample pump 192. Sample line flow may be measured using flow sensor 190.

Therapeutic gas delivery system controller may be configured to execute a program or algorithm which calculates run-time-to-empty using the values received by the therapeutic gas delivery system controller and/or stored in memory from a temperature sensor 130(a), 130(b), a gas pressure sensor 120(a), 120(b), therapeutic gas pressure regulator 122(a), 122(b), flow sensor 146(a), 146,(b), 166, and gas source identifier reader 131 (therapy gas concentration, either actual or target). To obtain the run-time-to-empty value, the volume of therapeutic gas in the therapeutic gas source at a selected time-point during therapy may be calculated using the Boyle's Law or the Ideal Gas Law and the wetted volume of the therapeutic gas source. That is, using the temperature of the therapeutic gas, the therapeutic gas pressure, and the known wetted volume of the therapeutic gas source 116(a), 116(b), the pressure of water vapor at the measured temperature is subtracted from total gas source pressure to obtain the pressure of the dry therapeutic gas. Boyle's Law (Vₐ=p_{c}V_{c}/pₐ) or the Ideal Gas Law (PV=nRT) is applied to calculate the volume in liters of the dry therapy gas at the measured temperature. In various embodiments, the run-time-to-empty may be calculate continuously to display changes in gas source pressure, intermittently, or when a delivery dose is set or changed to reflect changes in the run-time-to-empty for the new set dose.

In various embodiments, oscillating run-time-to-empty values may not be displayed. To avoid oscillating run-time-to-empty values being displayed intermittent recalculation may be implemented to avoid rapid changes in pressure and/or temperature, and allow a specific run-time-to-empty value to be displayed for a period of time sufficient for a user to read the run-time-to-empty value.

An average therapeutic gas consumption rate may be derived using data obtained from periodic and/or continuous measurements of a) average L/min. measured by the flow controller or commanded to the flow controllers over a period of time, b) average ventilation flow rate measured by BCG flow sensors 108(a) and/or 108(b) over a period of time, or c) set dose in ppm and an average ventilation flow rate measured by BCG flow sensors 108(a) and/or 108(b) over a period of time, which gives an average therapy gas flow rate in L/min. to be delivered.

By way of example, calculation of average therapeutic gas delivery/consumption rate using set dose and an average ventilation flow rate over a period of time is calculated as follows:

QNOset₍ₙ₎={YNOset/(YNOcyl−YNOset)}Qᵢ(n) (SLPM)

### Where

QNOset=NO flow rate desired (SLPM)
Qₐ=Injector Module flow rate (SLPM)
YNOset is the delivery set-point, the user set NO
concentration value (ppm)
YNOcyl is NO cylinder concentration (ppm)

Run-time-to empty (RTE) for the selected time-point is then calculated from the volume of therapy gas in the therapy gas source and the consumption rate calculated by one of the above methods:

RTE=(Remaining cylinder volume-reserve volume-known purge sequences)/(average therapy gas consumption rate(primary+secondary)+known leak rate).

In exemplary embodiments, algorithms may be executed (e.g., using the above calculation) by therapeutic gas delivery system 100 which may be configured to leave some amount of gas pressure (i.e., gas volume) in the therapeutic gas source 116(a), 116(b), etc. (the “reserve volume”) rather than running the gas source to empty. For example, the gas source may be a cylinder that may be deemed “empty” to the user when the cylinder pressure reaches 300 psi, 200 psi or 30 psi. This minimum pressure may be the minimum residual pressure needed for the regulator to function, plus pressure loss through valves, conduits, etc upstream of the pressure regulator, and/or plus pressure required for purging. Further, this may be used to compensate for therapeutic gas delivery system 100 being configured to be for a therapeutic gas source that always has a pressure of at least, or more than, 30 psi. In various embodiments, run-time-to-empty calculations may also take into account use of therapeutic gas for anticipated purges due to for example a low set dose/flow rate.

In exemplary embodiments, the therapeutic gas delivery system controller may be configured to automatically reduce the delivery dose to conserve gas when run-time-to-empty calculations indicate the operating therapeutic gas source(s) 116(a), 116(b) is getting low and there is no back-up therapeutic gas source(s) 116(a), 116(b) available to supply the therapeutic gas at a sufficient pressure. Further, to provide the lower dose, the therapeutic gas delivery system 100 could ignore or bypass the minimum pressure threshold for the gas source, and continue delivering the therapeutic gas until the therapeutic gas source(s) 116(a), 116(b) is empty. In such instances, alarms may be provided. The above may be beneficial as a lower dose may be safer than discontinuation of therapy, therefore a reduced dose may be provided to the patient. In various embodiments, therapeutic gas delivery system controller may be configured to automatically reduce the delivery dose to conserve gas when high NO₂ is detected to reduce the amount of NO available to react with O₂. In various embodiments, the therapeutic gas may be provided concurrently from two or more therapeutic gas source(s) 116(a), 116(b) to provide a larger total volume of therapeutic gas at the lower pressure(s) until empty.

In one or more embodiments, as described above, the therapeutic gas delivery system controller may communicate the calculated run-time-to-empty for the current set dose to a central display 102 and/or to a status display 112(a), 112(b) associated with a particular gas supply subsystem 110(a), 110(b) to notify the user of the run time remaining for the particular therapeutic gas source 116(a), 116(b) in a particular receptacle. When run-time-to-empty reaches predetermined levels, the therapeutic gas delivery system controller may also communicate modified alarms to the central display 102 and/or to a status display 112(a), 112(b) to indicate varying levels of criticality. For example a high level alarm may indicate that a half hour of run time remains, a moderate level alarm may indicate that an hour of run time remains, and a low level alarm may indicate that an hour and a half of run time remains. For another example, therapeutic gas delivery system controller may activate an audible alarm on the therapy gas delivery system or transmit an alarm to a wireless device (e.g., smart phone) to notify the user of remaining run-time. In one or more embodiments, a therapeutic gas delivery system comprising two or more therapeutic gas sources may supply therapeutic gas from the therapeutic gas source having the shorter run-time-to-empty value. In various embodiments, the therapeutic gas delivery system may seamlessly transition from a first therapeutic gas source to a second therapeutic gas source when the first therapeutic gas source has reached the intended run-time-to-empty value. In various embodiments, the calculated run-time-to-empty for the current set dose and/or the various alarm levels may be communicated to a hospital information system. Alarms may sound when the therapeutic gas delivery system 100 is operating on only one therapeutic gas source 116(a), 116(b). Alarms may be triggered based on the run-time-to empty value and/or therapeutic gas source 116(a), 116(b) pressure measured at gas pressure sensor 120(a), 120(b). Such alarms may be audible and/or visual. In at least some instances the run-time-to-empty may be the combined run-time-to-empty for both therapeutic gas sources, for example, depicted as one value and/or in any other visual format (e.g., graph, chart, image, etc.)

In various embodiments, display(s) 102, 112(a), 112(b), etc., may provide visual representation (e.g., graphical representation, bar graph, etc.) to a user visually indicating the remaining amount of therapeutic gas available from the therapeutic gas source 116(a), 116(b). This may be beneficial as the user may see when a therapeutic gas source 116(a), 116(b) will need to be replaced. A user may anticipate change-over from an active therapeutic gas source to a second (e.g., unused, full) therapeutic gas source by observing the actual RTE value, or visual representation, shown on display(s) 102, 112(a), 112(b). The visual representation may be displayed alongside the RTE value for each gas source, or instead of a RTE value when a dose is not set or flow through a flow control channel or an injector module 107 is not detected. In addition, an alarm may be provided when a therapeutic gas source is getting low, or the therapeutic gas delivery system 100 is down to only one operating therapeutic gas source. The therapeutic gas delivery system 100 may provide an alarm and/or instructions for the user to replace the depleted therapeutic gas source with a full therapeutic gas source. Displaying the actual RTE value(s) and/or visual indicators (e.g., bar graph, alarms, etc.) may allow the user to be aware of the remaining run time for the gas sources without having to look for the reading on a pneumatic pressure gauge attached to the gas source regulator and/or such visual displays may make monitoring the therapeutic gas delivery system 100 easier and help to avoid errors due to misreading various gauges and mechanical settings. Having one or more displays showing a run-time-to-empty value on the front of the system may mitigate problems associated with users having very little, or no, warning before the pressure supplied by a therapeutic gas source is unable to satisfy input pressure requirements for therapeutic gas pressure regulator 122(a), 122(b) and/or flow control valve(s) 143(a), 143(b), 163 and/or sensors. In various embodiments, the display(s) 102, 112(a), 112(b) may provide redundancy by being configured to allow a user to operate the therapeutic gas delivery system 100 from any of the displays 102, 112(a), 112(b), for example where each display is a touch screen that accepts user input.

In exemplary embodiments, implementation of two therapeutic gas sources 116(a), 116(b) provides redundancy, where second therapeutic gas sources 116(b) may supply therapeutic gas to a patient 203 when the first therapeutic gas sources 116(a) becomes depleted. For example, therapy gas delivery to the patient is initiated from therapy gas source 116(a) and delivered to the patient as described above. Further, as the run-time-to empty reaches a minimum value predetermined by the user and/or the system 100, therapeutic gas delivery system controller may close shut off valve 126(a) and open shut off valve 126(b) to source therapy gas delivery from second therapy gas source 116(b).

In one or more embodiments, therapeutic gas delivery system controller may automatically adjust for varying gas source concentrations when changing over from a first therapeutic gas source 116(a) to a second therapeutic gas source 116(b) containing the same therapeutic gas at a different concentration. By way of example, to accomplish the above gas source concentration information may be provided by gas source identifier 128(b), which may have recorded thereon the target and/or actual measured concentration of the therapeutic gas in therapeutic gas source 116(b). Further, as discussed above, gas source identifier 128(b) may also have recorded thereon additional data such as the identity of the therapy gas and/or its expiration date. In exemplary embodiments, use of the higher concentration therapeutic gas source may require system 100 increase in the average injector module 107 flow rate before delivery of the therapeutic gas would begin, or a reduction in therapeutic gas flow rate through flow control valve(s) 143(a), 143(b), 163, to maintain the same set dose to the patient 203. Similarly, injector module 107 flow rate may be reduced, and/or therapeutic gas flow rate through flow control valve(s) 143(a), 143(b), 163, may be increased to maintain the same set dose to the patient 203 for a lower therapeutic gas source 116(b) concentration.

If the therapeutic gases in therapeutic gas source 116(a) and therapeutic gas source 116(b) have different concentrations, therapeutic gas delivery system controller may automatically instruct a purge of the therapeutic gas delivery system, in which gas from the succeeding therapeutic gas source 116(b) is flushed through the high pressure side of the system, by opening purge valve 124(b) to evacuate all of the higher or lower concentration therapeutic gas from the manifold before opening second shut off valve 126(b) to the rest of the system, in addition to oxygen trapped that may form into NO₂. In exemplary embodiments, purging to the atmosphere may be through a dedicated purge port in fluid communication with purge valve(s) 124(a), 124(b) to prevent exposure of the patient to purged gases (e.g., wrong concentration, contaminated, NO₂, etc.).

In exemplary embodiments, therapeutic gas delivery system controller may adjust parameters accordingly in the therapeutic gas delivery algorithm calculations to maintain the desired set dose taking into account the therapeutic gas concentration in therapy gas source 116(b). In at least some instances, if the therapeutic gas in the succeeding therapy gas source 116(b) is different from the therapeutic gas in therapeutic gas source 116(a), therapeutic gas delivery system controller may automatically orchestrate, instruct orchestration of, a purge of the therapeutic gas supply subsystem 110(a), 110(b) before opening shut off valve 126(b) to evacuate all of the preceding therapy gas from the remainder of the system. Therapeutic gas delivery system controller may then adjust parameters accordingly in the therapy gas delivery algorithm for therapy gas source 116(b) to deliver the correct set dose to the patient.

Pre-Use Verification Processes and/or Verification Processes

In exemplary embodiments, at least some aspect of the present invention relate to systems, methods, and/or process for, amongst other things, performing pre-use verifications by confirming the proper operation of a therapeutic gas delivery system 100, leaks, the proper functioning of the gas supply subsystem(s), gas delivery subsystem(s), and/or gas analyzer subsystem(s), and by extension the proper functioning of the valve(s), flow sensor(s), pressure sensor(s), detector(s), regulator(s), and/or subsystem controller(s), to name a few. In some examples, the pre-use checkout may be completed in approximately five minutes. For example, the pre-use checkout may be completed in less than about 1 minute, 2 minutes, 3 minutes, 4 minutes, 5 minutes, 6 minutes, 7 minutes, 8 minutes, 9 minutes, or 10 minutes. The decreased amount of time for pre-use checkout may allow for improved and faster patient care. The faster the therapeutic gas delivery system 100 has been checked and is available for delivering the therapeutic gas, the faster the patient may be treated. This is particularly important when the patient is in a critical condition (e.g. neonatal infant).

With respect to at least pre-use verifications of the present invention, some found previous pre-use procedures to be difficult and intimidating, and required extensive training. Exemplary embodiments of the present invention reduce and/or simplify the number and sequence of pre-use procedures and/or increases patient safety by eliminating and/or mitigating risks associated with previous pre-use procedures. For example, abnormalities and/or failures of elements of system 100 may result in sudden discontinuation of therapeutic gas and thereby a sudden removal of therapy to a patient, which may result in potential life threatening hazard (e.g., rebound hypertension); however, using systems, methods, and processes for, amongst other things, performing pre-use verifications may result in detection of an abnormality and/or failure during the pre-use performance verification test and mitigation of a potentially life threatening hazard. For example, detection of an abnormality and/or failure during the pre-use performance verification may effectively convert a potential hazard from the sudden removal of therapy to a delay of therapy (e.g., time to get another device), which may be much less severe.

Purging of system 100 may be important as air/O₂/contaminants may enter into components of system 100 configured to fluidly communicate NO. This may be problematic as NO may react with this air/O₂/contaminants, for example, generating NO₂. These air/O₂/contaminants may enter system 100 via physical connection of therapeutic gas source 116(a), 116(b) to gas supply subsystem 110(a), 110(b), for example, trapping air/O₂/contaminants between the therapeutic gas source valve 117(a), 117(b) and the connection valve 118(a), 118(b).

In at least some instances, after properly receiving and/or verifying therapeutic gas source 116(a), 116(b), the therapeutic gas delivery system controller may initiate a purge sequence of the conduit/manifold between the therapeutic gas source valve 117(a), 117(b) and the closed shut off valve 126(a), 126(b), wherein the purged gas may exit the conduit/manifold via opened purge valve 124(a), 124(b). In various embodiments, a purge sequence may be initiated within a fraction of a second and/or within 2 seconds of detecting a properly received therapeutic gas source 116(a), 116(b). This purge may avoid the therapeutic gas from coming into prolonged contact with trapped air/O₂/contaminants introduced, for example, by the fluid connection between gas source valve 117(a), 117(b) and connection valve 118(a), 118(b).

In one or more embodiments, a conduit/manifold between connection valve 118(a), 118(b) and closed shut off valve 126(a), 126(b) may be purged by opening purge valve 124(a), 124(b) when therapeutic gas source 116(a), 116(b) is removed, for example, as indicated by gas source detector 132(a), 132(b). This purge may be used to reduce pressure between connection valve 118(a), 118(b) and closed shut off valve 126(a), 126(b), and/or evacuate stale gas from the manifold. As used herein, “stale” means that the therapeutic gas source may have reacted with air/O₂, unacceptable levels of NO₂ may have built up in the manifold, and/or other contaminants (e.g., H₂O, rust, etc.) may have entered the manifold or accumulated over time. The purge may lower the high pressure in the manifold back to just below a minimum cutoff of 200 PSI pressure (residual pressure), such that insertion of a new therapeutic gas source will trigger a higher pressure reading at gas pressure sensor 120(a), 120(b). Therapeutic gas delivery system 100 may not rely on gas pressure sensor 120(a), 120(b) to detect the presence of therapeutic gas source 116(a), 116(b) in fluid communication with the conduit/manifold 119(a), 119(b) because the response time of pressure sensor 120(a), 120(b) may be too slow to initiate a purge quickly enough to avoid gas reactions, and/or connection valve 118(a), 118(b) may have retained a gas pressure within conduit/manifold 119(a), 119(b) commensurate with the pressure in the mated therapeutic gas source 116(a), 116(b) that prevents a pressure change from being measured.

In one or more embodiments, purging sequences may be initiated, for example, by the therapeutic gas system controller, when therapeutic gas source 116(a), 116(b) is received (e.g., coupling member 114(a), 114(b) of therapeutic gas source 116(a), 116(b) mated with gas source coupling 115(a), 115(b); load handle (not shown) operatively manipulated; etc.) and/or during delivery of therapeutic gas to a patient.

Further to air/O₂/contaminants that may enter when therapeutic gas source 116(a), 116(b) is received (e.g., via physical connection of therapeutic gas source 116(a), 116(b) to gas supply subsystem 110(a), 110(b), etc.), low rates of NO consumption may trigger the need for purging sequences from the therapeutic gas source 116(a), 116(b) and all gas conduits/components in use. This build-up of NO₂ may also occur if oxygen permeation rate through the soft elastomer materials of conduits and/or seals is sufficient for NO gas volume moving through the system at a low rate to react causing the NO₂ conversion rate to increase. Conduit lengths, seals, and dead spaces may be reduced or eliminated to keep molecule of NO leaving the gas source and heading towards the patient circuit moving at the fastest rate practically possible to reduce dwell time. In at least some instances purging sequences may be more frequent when therapeutic gas consumption rates are low.

In at least some instances, purging sequences may be initiated during delivery of therapeutic gas to a patient because, for example, the delivery dose may be sufficiently low that the flow rate of therapeutic gas through one or more of the flow control channels is sufficiently low to allow a build-up of NO₂ on the high pressure side of shut off valve 126(a), 126(b), and/or the upstream side of primary flow control valve 143(a), 143(b), and/or secondary flow control valve 163. As described above, these purging sequences of the gas flow path to a vent (e.g., opened purge valve) removes the built-up NO₂ and other contaminants.

Similarly, purging sequences may be initiated when therapeutic gas delivery system 100, first gas supply subsystem 110(a) and/or second gas supply subsystem 110(b), have not been in use for a prolonged and/or predetermined amount of time (e.g., 10 min, 30 min, 1 hr, 6 hours, 12 hours, 24 hours, etc.). Purging sequences may utilize gas from a gas source (e.g., therapeutic gas source, etc.) and/or the purge may utilize pressurized gas contained between connection valve 118(a), 118(b) and a closed shut off valve 126(a), 126(b). Purging sequences described herein may be triggered upon detection of no therapeutic gas source, for example, as indicated by gas source detector 132(a), 132(b), load handle and/or gas source identification sensor 128(a), 128(b).

In various embodiments, purging sequences described herein may enable system 100 to maintain receptacle/gas supply subsystem 110(a), 110(b) primed for receiving therapeutic gas source 116(a), 116(b) and/or primed for seamlessly transition from one therapeutic gas source to another therapeutic gas source. As described above, seamless transition may be anticipated based on pressure and/or RTE calculation for the active (i.e., in use) therapeutic gas source 116(a), 116(b). Further, in exemplary embodiments, the duration and/or volume of gas used for purging sequences may be reduced (e.g., mitigate therapeutic gas waste, mitigate the amount of therapeutic gas purged/wasted into the surrounding environment, etc.). By way of example, orifices of the purge valves may be calibrated such that purge flow rates may be known, and therefore the volume of gas used for purging sequences may be dependent on purge valve open time.

In one or more embodiments, purging sequences may comprise a series of intermittent openings of purge valve 124(a), 124(b), and/or all of the flow control channel valves for a period of about 1 second to about 10 seconds followed by a period of about 1 second to about 10 seconds during which purge valve 124(a), 124(b), and/or all of the flow control channel valves, are closed. This intermittent opening and closing may be repeated 5, 10, 15, 20 times. In various embodiments, purging sequences may be increased to prime the therapeutic gas source for use more quickly, for example, by using the therapeutic gas in a continuous purge that may last from about 1 minute to about 10 minutes, or any time in between.

In one or more embodiments, therapeutic gas delivery system 100 may not power down until therapeutic gas sources 116(a), 116(b) are removed (e.g., released from receptacle/gas supply subsystem 110(a), 110(b), etc.). To at least prevent build-up of NO₂ and/or reduce waste of therapeutic gas, therapeutic gas delivery system 100 may require removal of therapeutic gas sources 116(a), 116(b) before shutting therapeutic gas delivery system 100 off. In at least some instances, an alarm may be provided until all therapeutic gas sources 116(a), 116(b) are removed. After removal of the therapeutic gas sources 116(a), 116(b), purging of the now empty bay(s) may be conducted, as described above. In various embodiments, a purge may not be initiated if the run-time-to-empty value indicates the therapeutic gas source is low (e.g., in a medium or high alarm state) in order to conserve therapeutic gas for delivery to the patient. In exemplary embodiments, when powered on, if therapeutic gas delivery system 100 detects a cylinder has been received the system may initiate a purge and/or alert.

In exemplary embodiments, purging sequences may be initiated to purge fluid pathways downstream of shut off valve 126(a), 126(b) such as conduits (e.g., conduit 101(a), 101(b), 172, etc.), flow control channels (e.g., flow control channels 141(a), 141(b), 161, etc.), and/or any other fluid pathways and/or components downstream of shut off valve 126(a), 126(b). By way of example, to purge downstream, shut off valve 126(a), 126(b) may be opened while purge valve 124(a), 124(b) is closed, enabling flow of therapeutic gas from the gas supply subsystem to at least one of the one or more flow control channels 141(a), 141(b), 161 and in turn to an egress from therapeutic gas delivery system 100 (e.g., purge valve, outlet from therapeutic gas delivery system 100, etc.). In various embodiments, primary gas delivery subsystem 140 and/or secondary delivery subsystem may include at least one purge valve in fluid communication with flow control channel 141(a), flow control channel 141(b), flow control channel 161(a), and/or at least one purge valve in fluid communication with injector module 107. In various embodiments, the corresponding shut off valves for each of the flow control channels may be selectively and/or sequentially opened and closed to purge the flow control channels. By way of example, when one flow control channel has been purged, the associated shut off valve 142(a), 142(b), 162, may be closed and the shut off valve for the next flow control channel may be opened.

In one or more embodiments, system 100 may perform pre-use verification procedures and/or during delivery of therapeutic gas to a patient for leaks by pressurizing, and/or prompting a user to install a therapeutic gas source, the gas supply subsystem at least between connection valve 118(a), 118(b) and closed shut off valve 126(a), 126(b) to a pressure above atmospheric pressure, monitoring the pressure between connection valve 118(a), 118(b) and closed shut off valve 126(a), 126(b) with gas pressure sensor 120(a), 120(b) for a predetermined time period, and presenting an alarm if the pressure between connection valve 118(a), 118(b) and closed shut off valve 126(a), 126(b) decreases greater than an expected amount over a predetermined time period (e.g., decrease in pressure due to a leak, etc.). In various embodiments, the predetermined time period may be a fixed time period, for example 30 seconds, 5 min, 10 min, 15 min, 20 min, 30 seconds or the time period may be between the initiation of a pre-use verification procedure and completion of the pre-use verification procedure, embodiments of which are described herein. In various embodiments, a greater than expected amount may be any drop in pressure over a short period (e.g., 5 min, 10 min, 15 min) or a drop in pressure larger than previously seen for a known leak-tight system and/or tested system for longer periods of time (e.g., 30 seconds, 20 min, 30 min, time for check-out, etc.).

In at least some embodiments, system 100 may perform checks for leaks within system 100, for example, during pre-use verification and/or when delivering therapeutic gas (e.g., when delivering therapeutic gas to a patient, etc.). Similar to checking for leaks between connection valve 118(a), 118(b) and closed shut off valve 126(a), 126(b), system leaks may be identified by pressurizing, and/or prompting a user to install a therapeutic gas source, the system to a known pressure (e.g., pressure above atmospheric pressure, etc.), opening and/or closing valves within system 100 and monitoring the pressure between the various open and/or closed valves with pressure sensors. Further, in at least some instances, system 100 may perform checks for leaks within system 100 when delivering therapeutic gas (e.g., background leak checks) by monitoring pressure sensors affiliated with system 100 for decreases in pressure that are greater than an expected amount over a predetermined time period.

In at least some embodiments, checks for leaks performed by system 100 may factor in the therapeutic gas used for pre-use verification from both gas sources, purging, etc.

In one or more embodiments, gas flow rate measured at each of delivery flow sensors 146(a), 146(b), 166 may be compared against the gas flow rate through confirmatory flow sensors 148(a), 148(b), 168 in series with delivery flow sensors 146(a), 146(b), 166 for the associated flow control channel. In various embodiments, an alarm, recommend replacing at least one of the sensors, perform verification processes (described below in greater detail) to confirm which sensor is not functioning properly, and/or provide flow information from the functioning flow sensor, etc. may be provided if there is a discrepancy between the gas flow rate through the delivery flow sensor and the gas flow rate through the confirmatory flow sensor, where a discrepancy greater than a threshold amount of about 10%, or about 7%, or about 5%, or about 2.5%, or about 2%, or about 1%, or about 0.5% triggers an alarm.

Aspect of the present invention relates to a method of confirming the proper functioning of gas delivery and injector module operation. In certain embodiments, therapeutic gas delivery system controller may further comprise an automated performance verifications during delivery of therapeutic gas and/or pre-use performance verification algorithm that purge at least a portion of therapeutic gas delivery system 100 upon installation of therapeutic gas source 116(a), 116(b) and/or during delivery of therapeutic gas, and verifies operability of selected components of therapeutic gas delivery system 100 before use (e.g., pre-use) and/or during use (e.g., during delivery of therapeutic gas).

In one or more embodiments, pre-use performance verification and/or performance verification during delivery of therapeutic gas may comprise the therapeutic gas delivery system controller comparing the concentration of the therapeutic gas reported by the gas analyzing subsystem 180 to the ratio-metric calculation(s) based on the flow rate values reported by flow sensors 146(a), 146(b), 166, 148(a), 148(b), 168 for each of flow control channels 141(a), 141(b), 161. A result of the comparison showing a different gas analyzer value for one flow control channel may indicate that the flow control valve, sensor, and/or component associated with that flow control channel is not functioning properly, whereas a different gas analyzer value compared to the ratio-metric value for all flow control channels may indicate that the therapeutic gas sensor is out of calibration. The use of redundant flow sensors 146(a), 146(b), 166, 148(a), 148(b), 168 in each of the flow control channels allows the system and/or user to pinpoint which component may not be functioning through cross checking. In this manner, it may be determined if a flow valve 143(a), 143(b), 163 needs calibration or the gas analyzing subsystem 180 needs high calibration. In various embodiments, gas analyzer values and/or ratio-metric values within pre-set tolerance (e.g. +−20% of set dose) may be considered an acceptable variation. The redundant ratio-metric calculations for flow control channels 141(a), 141(b), 161 may provide a basis to correct the output of the gas analyzer without the need for calibration gas if the ratio-metric calculations are all in agreement with one another. The difference between the calculated ratio-metric values and the measured gas analyzer value indicates the amount by which the gas analyzer is out of calibration. The output of the gas analyzer may then be compensated for. The gas analyzing subsystem 180 may reference room air to prevent over-saturation during measurements. If a failure or error is detected at the gas analyzer, then rather than lose monitoring the device may display the ratio-metric delivered NO concentration from delivery or spy sensors in place of the gas analyzer measured NO concentration and alert the user of the issue.

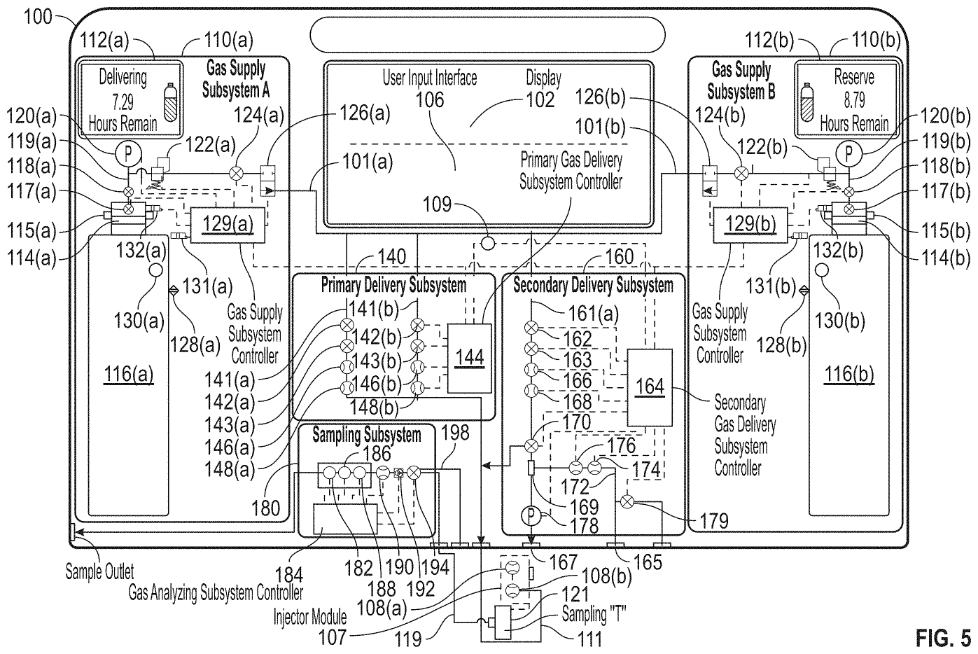

In various embodiments, a user may be instructed to connect the injector module 107 with a particular orientation to the low pressure outlet port 167 to test the injector module and secondary gas delivery subsystem 160, as shown for example in FIG. 5. In various embodiments, an alarm, recommendation of replacing at least one of the sensors, perform verification processes (described below in greater detail) to confirm which sensor is not functioning properly, and/or flow information from the functioning flow sensor, etc. may be provided if the breathing gas flow rates measured at low pressure confirmatory flow sensor 176, low pressure delivery flow sensor 174, injector module confirmatory flow sensor 108(b), or injector module delivery flow sensor 108(a) differs from the other measured breathing gas flow rates by greater than a threshold amount, where the threshold amount may be a difference of about 10%, or about 7%, or about 5%, or about 2.5%, or about 2%, or about 1%, or about 0.5% between two measured flow rates, or between any one of the sensor measured values and the average flow rate. The threshold amount may depend on the accuracy and tolerances of the flow sensors used in the system.

In various embodiments, pre-use performance verification and/or performance verifications during delivery of therapeutic gas may further comprise adjusting flow control valve 163 to provide a stream of therapeutic gas at an intended therapeutic gas flow rate; and determining if flow control valve 163 is properly functioning, where the subsystem flow control valve is in fluid communication with the low pressure outlet port. In various embodiments, a subsystem flow control valve may be adjusted to be completely open to provide the stream of therapeutic gas at a maximum therapeutic gas flow rate.

In one or more embodiments, the combined therapeutic gas flow rate and breathing gas flow rate may be measured at injector module delivery flow sensor 108(a) and injector module confirmatory flow sensor 108(b) in fluid communication with low pressure outlet port 167; and flow regulating valve 170 may be switched to divert the stream of therapeutic gas to an alternative flow path, where the flow regulating valve is upstream from and in fluid communication with the low pressure outlet port, and the subsystem flow control valve is upstream from and in fluid communication with the flow regulating valve 170, to determine if flow regulating valve 170 functioned properly by determining if the combined therapeutic gas flow rate and breathing gas flow rate decreased by the therapeutic gas flow rate when the flow regulating valve was switched to the alternative flow path. In various embodiments, the breathing gas flow rate may be measured at injector module delivery flow sensor 108(a) and injector module confirmatory flow sensor 108(b). In an exemplary embodiment, flow control valve 163 may be set to the highest flow rate, and a step change (e.g., increase) may be observe on injector module delivery flow sensor 108(a) and injector module confirmatory flow sensor 108(b). When the flow regulating valve 170 is switched to divert the gas flow from injector module 107, a decrease in gas flow rate may be detected downstream by injector module delivery flow sensor 108(a) and injector module confirmatory flow sensor 108(b). Similarly, when subsystem flow control valve 163 is set to a minimum or zero flow rate, a decrease in gas flow rate may be detected downstream by injector module delivery flow sensor 108(a) and injector module confirmatory flow sensor 108(b). This may be repeated several times. In some examples, the flow regulating valve 170 may be directional valve or a three-way valve.

In various embodiments, a flow rate may be measured at two or more secondary delivery subsystem flow sensors, wherein flow sensors 166, 168 are upstream from and in fluid communication with flow regulating valve 170; and the flow rates measured at each of the two or more subsystem flow sensors may be compared to determine if the two or more subsystem flow sensors are in agreement. In various embodiments, therapeutic gas blending ratio may be calculated from the measured flow rate measured by at least one of the two or more subsystem flow sensors and from the breathing gas flow rate measured by the low pressure delivery flow sensor; and comparing the calculated therapeutic gas blending ratio to the measured concentration of therapeutic gas exiting the injector module.

In various embodiments, each of the one or more shut off valves and/or flow control valves for each of the one of the one or more flow control channels may be selectively and/or sequentially opened and closed to confirm functionality and/or deliver a controlled flow of therapeutic gas to the injector module. In various embodiments, the gas analyzer confirms flow control channel(s) 141(a), 141(b) are functioning properly and providing the intended dose. Measurement of flow rates by redundant flow sensors may detect discrepancies between the flow controllers, flow sensors, and/or flow control channels. A purge of each flow control channel and delivery conduit 111 may also occur while the confirmation of flow control is being conducted. In various embodiments, the gas analyzer subsystem may reference room air while the purge is occurring.

In an alternative scenario the gas analyzer may be able to select to sample from within a pre-use verification port, so that the sample line does not need to be connected during performance verification.

In one or more embodiments, therapeutic gas may be delivered to the injection port of the injector module 107 through delivery conduit 111, a gas sample may be collected by sample tee 121 and directed to the gas analyzer to confirm the gas flow rate of therapeutic gas through flow control channel(s) 141(a), 141(b) provides an intended dose.

In various embodiments, system 100 may automatically compensate for different therapeutic gas source concentrations, for example, in response to pre-use verification. By way of example, system 100 may adjust flow valve 163 output during the performance verification to reduce the flow rate to half if the therapeutic gas source concentration is doubled.

In various embodiments, the system may instruct a user to disconnect the injector module from the low pressure outlet port and connect the injector module to ventilator breathing circuit inspiratory limb 213. In various embodiments, the direction of gas flow from a ventilator through the injector module may be confirmed by bi-directional flow sensors 108(a), 108(b) of injector module 107.

In various embodiments, the system may instruct a user to disconnect the main electrical feed to therapeutic gas delivery system 100 to check that the backup battery is charged and functioning.

In various embodiments, the system may go through a post-use/shut-down verification procedure which may comprise relaying patient information data to the medical facility's information system.

In various embodiments, the system may prompt a user to remove therapeutic gas source(s) 116(a), 116(b), and verify that therapeutic gas source(s) 116(a), 116(b) have been removed through gas source detector 132(a), 132(b). At such time, the system may go through a shut-down purge as discussed above.

In various embodiments, the system may prompt a user to clean injector module 107 and/or provide instructions for cleaning injector module 107. In various embodiments, the system may prompt a user if the system is due for service.

One or more embodiments of the present invention provide an exemplary pre-use performance verification procedures, in which the following steps and/or procedures may be performed to ensure the proper functioning of a therapeutic gas delivery system 100; determine if there are leaks; ensure the proper functioning of the gas supply subsystem(s), gas delivery subsystem(s), and/or gas analyzer subsystem(s), and by extension the proper functioning of the valve(s), flow sensor(s), pressure sensor(s), detector(s), regulator(s), and/or subsystem controller(s). However, it is to be understood that any of these steps may be omitted or performed in a different order, or additional steps may be performed in addition those specifically indicated below. Furthermore, some of these steps may be performed concurrently, particularly if the steps are performed by components in separate subsystems and/or at least some of these steps may be performed during delivery of therapeutic gas to a patient.

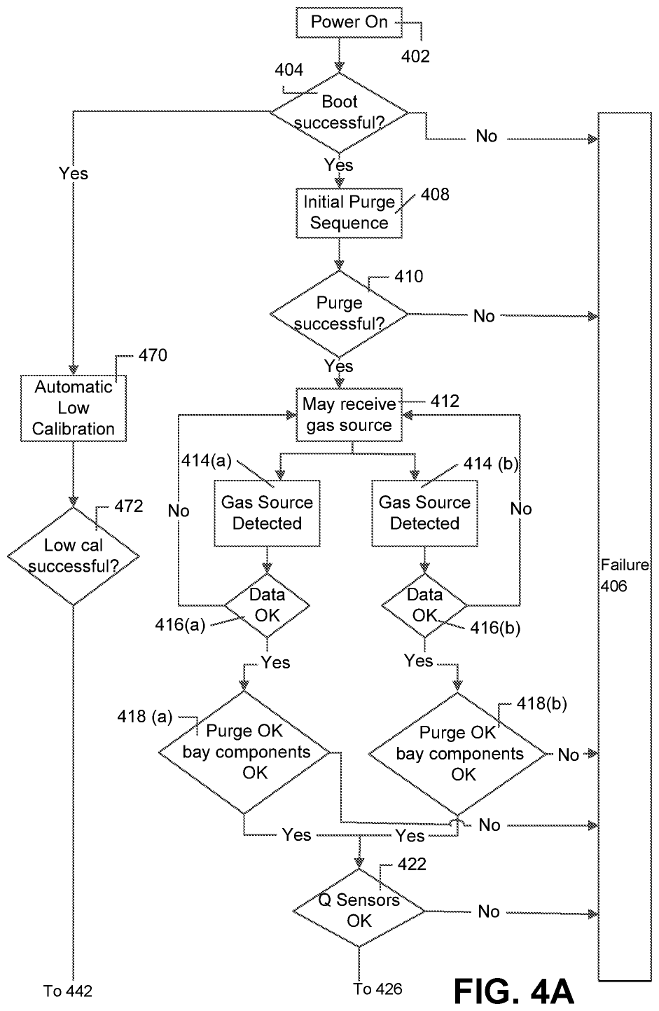

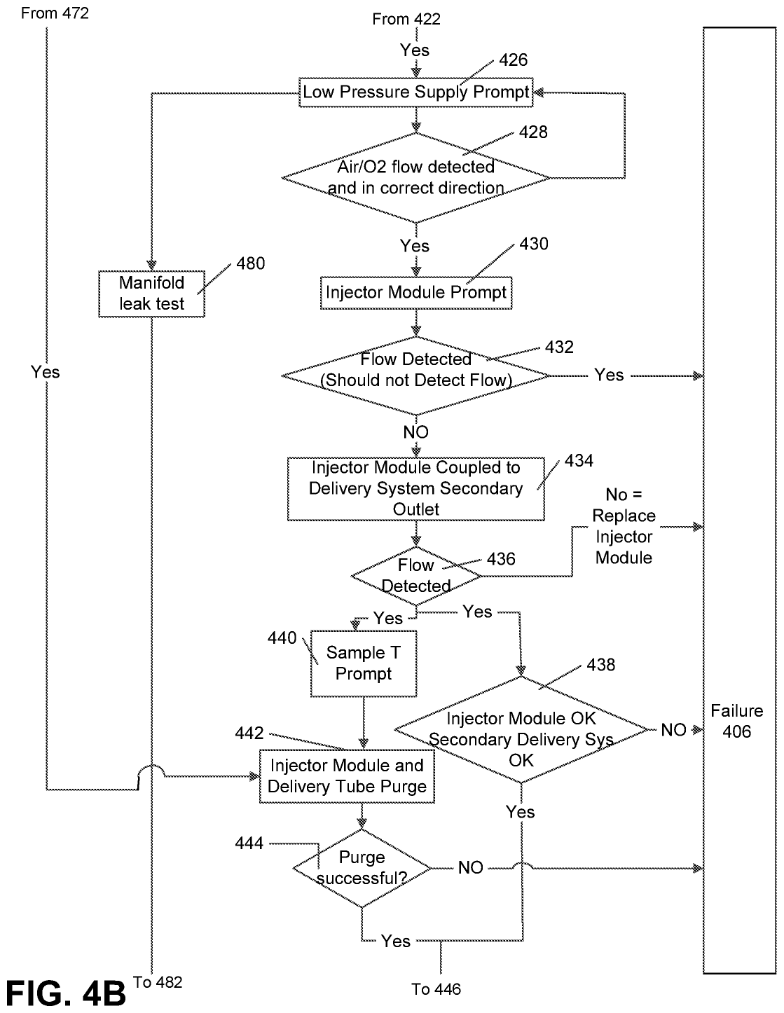

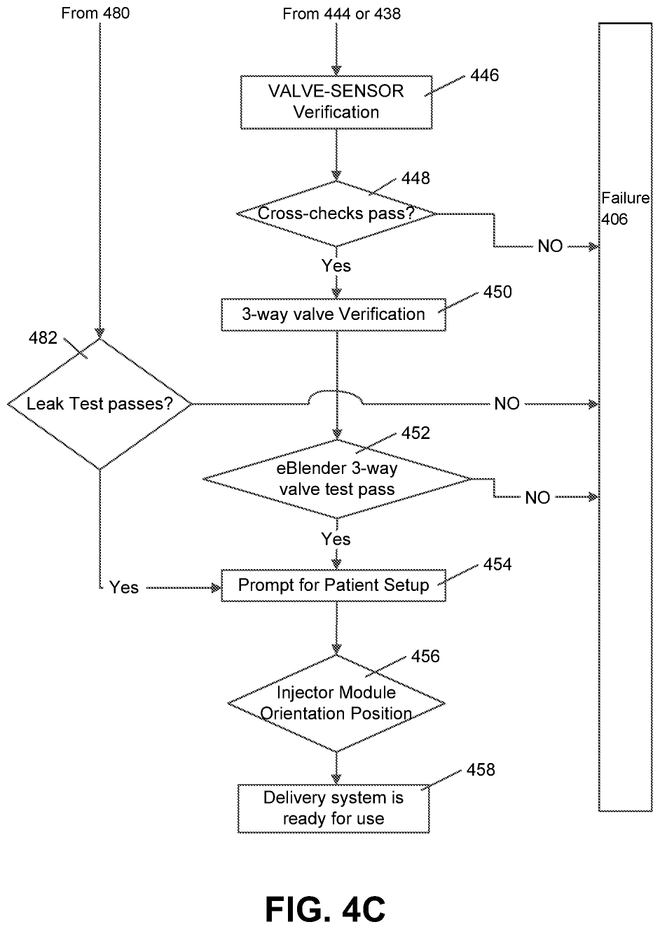

Referring to FIGS. 4A-4C, an exemplary pre-use performance verification procedure is depicted. At step 402, therapeutic gas delivery system 100 is started up (e.g., powered on by user, etc.). When started up, any and/or all subsystems (e.g., first gas supply subsystem 110(a), second gas supply subsystem 110(b), primary gas delivery subsystem 140, secondary gas delivery subsystem 160, and/or gas analyzing subsystem 180, etc.) may be booted up. At step 404, therapeutic gas delivery system 100 may confirm whether proper boot up of each subsystem occurred. If all subsystems properly boot then an initial purge sequence may begin, at step 408, and the purge may be verified as being successful, at step 410.

If any and/or all performance verifications process fail therapeutic gas delivery system 100 may undergo failure processes, at step 406, wherein therapeutic gas delivery system 100 may alarm the user (e.g., alarm provided on central display 102, user input interface 106, alarm provided on displays 112(a), 112(b), etc.), log the failure (e.g., store information in memory affiliated with system 100, for example, in an error log), indicate the source of failure and/or recommend a course of action (e.g., change setup, change component, etc.), shut down system if failure is critical, continue the performance verification process, and/or allow delivery of therapeutic gas to the patient, to name a few.

In exemplary embodiments, an initial purge sequence may be initiated by system 100, wherein residual pressure gas and/or gas in system 100 may be purged (e.g., via purge valves, via outlets, etc.). Residual pressure and/or gas may be from therapeutic gas sources that were previously received by therapeutic gas delivery system 100. For example, previously received therapeutic gas sources may be from a previous use of system 100 and/or from a user inserting a therapeutic gas source prior to turning on therapeutic gas delivery system 100. If the initial purge sequence was not successful, then therapeutic gas delivery system 100 may proceed to failure processes, at step 406. If the initial purge sequence is successful, therapeutic gas delivery system 100 may then receive the therapeutic gas source, at step 412, for example, as described above.

For ease, the exemplary pre-use performance verification procedures are depicted as being for two cylinders. This is merely for ease and is in no way meant to be a limitation. Similar techniques are envisioned for therapeutic gas delivery systems capable of receiving therapeutic gas from any number of sources.

At step 414(a), 414(b), received therapeutic gas sources may be detected by therapeutic gas delivery system 100 (e.g., using the techniques described above). In one or more embodiments, therapeutic gas source 116(a), 116(b) may be received by receptacle/gas supply subsystem 110(a), 110(b). To be received by receptacle/gas supply subsystem 110(a), 110(b), coupling member 114(a), 114(b) of therapeutic gas source 116(a), 116(b) may be required to mate with gas source coupling 115(a), 115(b) of receptacle/gas supply subsystem 110(a), 110(b). After being received, therapeutic gas source 116(a), 116(b) may be actuated (opened) thereby placing therapeutic gas source 116(a), 116(b) in fluid communication with gas pressure sensor 120(a), 120(b), which measures the pressure of the gas in therapeutic gas source 116(a), 116(b).

In one or more embodiments, therapeutic gas source 116(a), 116(b) may be automatically detected when a load handle (not shown) is operatively manipulated to release and/or lock therapeutic gas source 116(a), 116(b) with gas supply subsystem 110(a), 110(b) and/or gas source detector 132(a), 132(b) detects a therapeutic gas source. In various embodiments, gas source detector 132(a), 132(b) may be operatively associated with the load handle, where gas source detector 132(a), 132(b) detects when a load handle has been operatively manipulated. In various embodiments, gas source detector 132(a), 132(b) may be operatively associated with the gas source coupling 115(a), 115(b), where the gas source detector 132(a), 132(b) detects when matching coupling member 114(a), 114(b) of therapeutic gas source 116(a), 116(b) has been mated with the gas source coupling 115(a), 115(b).

At step 416(a), 416(b), data may be read in to confirm the correct cylinder has been received, for example, using the techniques described above. In exemplary embodiments, when received by therapeutic gas delivery system 100, gas source identifier reader 131(a), 131(b) may read gas source identifier 128(a), 128(b), which has recorded thereon the actual measured concentration of the therapeutic gas in gas source 116(a), 116(b) and/or the manufacturer's target gas concentration for therapeutic gas source 116(a), 116(b). Gas source identifier 128(a), 128(b) may also have recorded thereon additional data such as, but not limited to, the wetted volume of the gas source, the identity of the therapeutic gas, and/or its expiration date, to name a few. Data recorded on gas source identifier 128(a), 128(b) and gas pressure measured by gas pressure sensor 120(a), 120(b) may be communicated to therapeutic gas delivery system controller and stored in memory.

In various embodiments, the therapeutic gas delivery system controller may maintain shut off valve 126(a), 126(b) in a closed state until completion of verification analysis of the therapeutic gas source data, and keep therapeutic gas source closed off from the gas delivery subsystems downstream from shut off valve 126(a), 126(b) if incorrect information is detected (e.g. expired gas source, concentration out of range, wetted volume out of range, wrong therapeutic gas, etc.)

In one or more embodiments, the therapeutic gas delivery system controller may prompt a user to install a therapeutic gas source if an incorrect therapeutic gas source is received. By way of example, the presence of a correct or incorrect therapeutic gas source 116(a), 116(b) received by gas supply subsystem 110(a), 110(b) may be determined by analyzing therapeutic gas source data on and/or affiliated with gas source identifier 128(a), 128(b), which may be received by gas source identifier reader 131(a), 131(b). In exemplary embodiments, at any time during use (e.g., during pre-use verification procedures, during delivery of therapeutic gas to a patient, etc.), data (e.g., therapeutic gas source data) on and/or affiliated with gas source identifier 128(a), 128(b) may be analyzed, for example, by the therapeutic gas delivery system controller, to determine if the wrong therapeutic gas is coupled to the system, the therapeutic gas is expired, the therapeutic gas is the wrong concentration, the therapeutic gas source contains the correct therapeutic gas, the therapeutic gas is at sufficient pressure, etc.

In at least some embodiments, the therapeutic gas delivery system controller may prompt a user to install a therapeutic gas source if the received therapeutic gas source is determined to be empty and/or does not meet the minimum threshold (e.g., minimum threshold pressure). By way of example, the therapeutic gas delivery system controller may detect that gas supply subsystem 110(a), 110(b) is empty and/or does not meet the minimum threshold pressure using information communicated from gas pressure sensor 120(a), 120(b) indicative of the pressure of a received therapeutic gas source 116(a), 116(b).

In one or more embodiments, therapeutic gas delivery system 100 detects when a therapeutic gas source is installed and reads the affiliated information from the therapeutic gas source identifier attached to the gas source. In various embodiments, the therapeutic gas delivery system will confirm that the information from the therapeutic gas source identifier matches the expected identifier characteristics of the therapeutic gas. In exemplary embodiments, if the affiliated information from the therapeutic gas source identifier is found acceptable, the therapeutic gas delivery system may initiate a performance verification process during delivery of therapeutic gas.

At step 418(a), 418(b), after properly receiving and/or verifying therapeutic gas source 116(a), 116(b), the therapeutic gas delivery system controller may purge the system; verify the purge was successful by, for example, analyzing the concentration of the therapeutic gas and/or measuring the current detected through valves; and/or check all other related therapeutic gas delivery system components. If not successful and/or checks of other related components fail then therapeutic gas delivery system 100 may proceed to failure processes, at step 406.

At step 422, any and/or all flow sensors (e.g., flow sensors and corresponding confirmatory flow sensors, etc.) may be verified as no flow measurements should be seen because no gas flow has been initiated. If flow is measured (e.g., when no flow should be measured), therapeutic gas delivery system 100 may proceed to failure processes, at step 406, for example, as this may be indicative of a leak and/or sensor failure.

At step 426, therapeutic gas delivery system 100 may prompt users to attach a low pressure gas supply to the low pressure inlet port and, in at least some instances, set the low pressure gas supply flow to a known flow rate (e.g., 10 SLPM, etc.).

At step 428, flow may be detected and if flow is measured in the wrong direction (e.g., user attached low pressure supply to the low pressure outlet port, etc.) the user may be prompted re-attach the low pressure gas supply (e.g., returning to step 426). In exemplary embodiment, a flow of air/O₂ should be detected by the low pressure delivery flow sensor 174 and the low pressure confirmatory flow sensor 176. In various embodiments, the air/O₂ flow source to check the flow sensors 174, 176, 108(a), 108(b) may be air/O₂ from a regulated wall supply, a compressed gas cylinder supply, or a pump, which may be internal or external to the therapeutic gas delivery system 100. A pump, regulated wall supply, or compressed gas cylinder supply may be connected and/or activated by user. A pump may provide waveforms to test the dynamic measurement range of the flow sensors. Bi-directional pass-thru sensors may verify correct setup of air/O₂ inlet connection for the performance verification.

In various embodiments, low pressure outlet port 167 is used for both connection to an assisted breathing apparatus for delivery of therapeutic gas and/or for connection of an injector module 107 for the pre-use verification procedure. Use of the same low pressure outlet port 167 for both functions provides a means to simplify (e.g., reducing and/or eliminating operator error, etc.) the pre-use verification procedures with fewer user steps for check-out of primary delivery, backup delivery and monitoring systems. Low pressure outlet port 167 may also serve as storage location for injector module 107 by providing a known and obvious location for the injector module to be located when not in use. In various embodiments, low pressure inlet port 165 and low pressure outlet port 167 may comprise connectors, for example quick disconnect gas connectors, hose barb connectors, and hose couplings, or the low pressure outlet port 167 comprise an adaptor configured and dimensioned to connect directly to the injector module. In various embodiments, a disposable and/or sterilizable adapter that connects to the injector module may be used to connect to low pressure outlet port 167 for the performance verification. This allows for separation of the device, which is not sterilized, and injector module 107 which may be sterilized.

At step 430, therapeutic gas delivery system 100 may prompt users to attach the injector module 107 such that, at step 432, no flow should be seen by injector module delivery flow sensor 108(a) and/or injector module confirmatory flow sensor 108(b). For example, the user may be prompted to place the injector module in electrical communication with therapeutic gas delivery system 100 while the injector module is not exposed to gas flow. If flow is detected by injector module delivery flow sensor 108(a) and/or injector module confirmatory flow sensor 108(b) the user may be instructed to replace the injector module as one of the flow sensors may be working improperly, for example, at step 406.

At step 434, therapeutic gas delivery system 100 may prompt users to attach the injector module 107 to low pressure outlet port 167, as depicted in FIG. 5, such that low pressure flow may be detected by at least injector module delivery flow sensor 108(a) and/or injector module confirmatory flow sensor 108(b), at step 436. For example, a user may be instructed to attach the injector module 107 to the low pressure outlet port for testing. If flow is not detected by injector module delivery flow sensor 108(a) and/or injector module confirmatory flow sensor 108(b) the user may be instructed to replace the injector module as one of the flow sensors may be working improperly, for example, at step 406. In various embodiments, the direction of gas flow through the injector module may be determined by bi-direction flow sensors 108(a), 108(b).

In one or more embodiments, performance verification may comprise attaching an injector module at low pressure outlet port 167; attaching a low pressure gas supply to low pressure inlet port 165, where the low pressure gas supply (e.g., regulated hospital wall outlet/external supply/cylinder) provides a flow of breathing gas at a breathing gas flow rate, and where the low pressure inlet port is in fluid communication with the low pressure outlet port; measuring the breathing gas flow rate from the low pressure gas supply at low pressure delivery flow sensor 174 and/or at low pressure confirmatory flow sensor 176, where low pressure delivery flow sensor 174 and low pressure confirmatory flow sensor 176 are in fluid communication with the low pressure inlet port and the low pressure outlet port; measuring the breathing gas flow rate from the low pressure gas supply at injector module delivery flow sensor 108(a) and/or injector module confirmatory flow sensor 108(b), wherein the injector module delivery flow sensor and the injector module confirmatory flow sensor are in fluid communication with low pressure outlet port 167; and determining if one of the breathing gas flow rates measured at low pressure confirmatory flow sensor 176, low pressure delivery flow sensor 174, injector module confirmatory flow sensor 108(b), or injector module delivery flow sensor 108(a) differs from the other measured breathing gas flow rates by greater than a threshold amount. Air/O₂ flow rate may be in the range of about 0-60 SLPM, and may be detected as flowing in a forward direction. Placing the delivery flow sensors (e.g. injector module sensors and flow sensors) and confirmatory flow sensors in series facilitates detection of a single flow sensor in the fluid flow path that is not working and/or providing readings that do not match the others.

In exemplary embodiments, therapeutic gas delivery system 100 may determine when injector module 107 has been coupled to low pressure outlet port backwards. This may be accomplished because, amongst other things, the injector module delivery flow sensor and/or the injector module confirmatory flow sensor may be bi-directional flow sensors configured to determine the direction of gas flow through the injector module 107. In various embodiments, the injector module delivery flow sensor and the injector module confirmatory flow sensor are arranged in located relative to each other in series, parallel, skewed, and/or any other configuration.

At step 438, in exemplary embodiments, therapeutic gas delivery system 100 may deliver air/O₂ through secondary gas delivery subsystem 160 (e.g., received from a therapeutic gas source) to injector module 107 to at least verify injector module delivery flow sensor 108(a), injector module confirmatory flow sensor 108(b), flow sensor 174, and/or confirmatory flow sensor 176. In this configuration, the same flow of gas should be detected by each of injector module delivery flow sensor 108(a), injector module confirmatory flow sensor 108(b), flow sensor 174, and/or confirmatory flow sensor 176. If any flow sensors are found to not be functioning properly (e.g., measuring a different flow rate than two other flow sensors) then therapeutic gas delivery system 100 may undergo failure processes, at step 406.

By way of example, in exemplary embodiments, after performance verification has confirmed that injector module confirmatory flow sensor 108(b) and injector module delivery flow sensor 108(a) are both functioning properly, and low pressure delivery flow sensor 174 and low pressure confirmatory flow sensor 176 are both functioning properly, secondary delivery flow sensor 166 and secondary confirmatory flow sensor 168 may be tested. In various embodiments, the gas flow rate may be measured by secondary delivery flow sensor 166 and secondary confirmatory flow sensor 168 and compared to the incremental gas flow rate measured by injector module delivery flow sensor 108(a) and injector module confirmatory flow sensor 108(b).

At step 440, therapeutic gas delivery system 100 may prompt users to attach the gas sampling downstream from the injector module, as illustrated in FIG. 5. For example, a user may be instructed to attach a sample-T to the outlet of injector module 107, where the sample-T may divert at least a portion of the gas exiting injector module 107 to the gas analyzing subsystem 180. The sample-T may be downstream from injector module 107 flow sensor(s) 108(a), 108(b).

At step 442, in various embodiments, flow of the therapeutic gas through one or more of flow control channels 141(a), 141(b), 161 may purge air out of delivery conduit 111, injector module 107, and/or any conduits upstream from and/or in fluid communication with delivery conduit 111 and/or injector module 107. For example, therapeutic gas delivery system 100 may purge delivery conduit 111, injector module 107, and/or any conduits upstream from and/or in fluid communication with delivery conduit 111 and/or injector module 107 by providing therapeutic gas from flow control channel 141(a) and/or any other flow control channel. In exemplary embodiments, during purges the gas analyzer may reference room air (e.g., mitigate exposure to high concentration NO, perform calibration, etc.).

At step 444, therapeutic gas delivery system 100 may confirm if the purge, at step 442, was successful by detecting the purge with any of the flow sensors in fluid communication with the conduit where the purge flowed therapeutic gas through and/or by taking a sample of the purge flow, via the sample T connected to the injector module, using the gas analyzing subsystem 180. If not successful then therapeutic gas delivery system 100 may proceed to failure processes, at step 406.

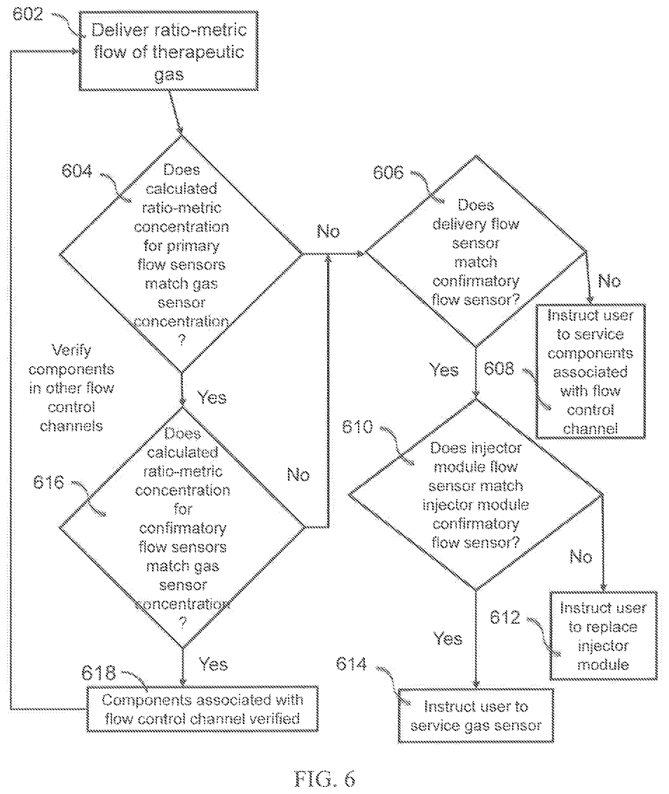

At step 446, in exemplary embodiments, therapeutic gas delivery system 100 may perform verification processes of any and/or all flow sensors affiliated with first gas supply subsystem 110(a), a second gas supply subsystem 110(b), a primary gas delivery subsystem 140, a secondary gas delivery subsystem 160, and/or a gas analyzing subsystem 180. By way of example, referring to FIG. 6, one or more embodiments of the present invention provide an exemplary processes (e.g., triangulation of failure that may be used for pre-use performance verification, triangulation of failure that may be used for performance verification during delivery of therapeutic gas, etc.) for determining whether various sensors are properly calibrated by cross-checking with other sensors.

At step 602, the therapeutic gas delivery system 100 delivers a ratio-metric flow of therapeutic gas according to a dose set by the user or according to a predetermined dose that is part of the pre-use performance verification procedure. Of course, similar techniques may be used for performance verification during delivery of therapeutic gas. The ratio-metric flow may be provided by the components in fluid communication with first primary flow control channel 141(a) (e.g. first primary control valve 143(a), first primary delivery flow sensor 146(a) and first primary confirmatory flow sensor 148(a)), the components in fluid communication with second primary flow control channel 141(b) (e.g. second primary control valve 143(b), second primary delivery flow sensor 146(b) and second primary confirmatory flow sensor 148(b)), the components in fluid communication with secondary flow control channel 161(a) (e.g. secondary flow control valve 163, secondary delivery flow sensor 166, and secondary confirmatory flow sensor 168)), etc. In one or more embodiments, components associated with one flow control channel is operated and verified, followed by operation and verification of a second set of components, followed by operation and verification of a third set of components, etc., until all relevant components have been verified.

At step 604, the primary delivery subsystem controller 144 and/or secondary gas delivery subsystem controller 164 compares the NO concentration measured by gas sensor 182 to the ratio-metric concentration calculated using the therapeutic gas flow reported by the delivery flow sensor 146(a), 146(b), 166, the NO concentration in the gas cylinder, the breathing gas flow reported by injector module delivery flow sensor 108(a), and/or flow sensor 174, 176. By way of example, the ratio-metric concentration for a given set of sensors is calculated as follows:

YNOcalc=(QNOmeasYNOcyl)/(QNOmeas+Qi)

### Where

YNOcalc=calculated ratio-metric NO concentration (ppm)
QNOmeas=measured NO flow rate (SLPM)
YNOcyl=NO cylinder concentration (ppm)
Qᵢ=injector module flow rate (SLPM)

In the above equation, QNOmeas may be provided by first primary delivery flow sensor 146(a), second primary delivery flow sensor 146(b), first primary confirmatory flow sensor 148(a), second primary confirmatory flow sensor 148(b), secondary delivery flow sensor 166 or secondary confirmatory flow sensor 168, and Qᵢ may be provided by injector module delivery flow sensor 108(a), injector module confirmatory flow sensor 108(b), flow sensor 174 or flow sensor 176, depending on which flow sensors are being verified.

If the calculated ratio-metric concentration does not match the NO concentration measured by gas sensor 182, then the flow information from delivery flow sensor 146(a), 146(b), 166 is compared to the flow information from its respective confirmatory sensor 148(a), 148(b), 168 at step 606. If the flow information from delivery flow sensor 146(a), 146(b), 166 does not match the flow information from confirmatory sensor 148(a), 148(b), 168, then step 608 provides that the user may be instructed to service the components in fluid communication with the flow control channel being verified, which includes the delivery flow sensor 146(a), 146(b), 166, the respective confirmatory sensor 148(a), 148(b), 168 and/or respective the control valve 143(a), 143(b), 163. Furthermore, if during therapy, the device may fail over to an alternate flow control channel or secondary delivery subsystem. If the flow information from delivery flow sensor 146(a), 146(b), 166 matches the flow information from confirmatory sensor 148(a), 148(b), 168, then the flow information from injector module delivery flow sensor 108(a) or flow sensor 174 is compared to the flow information from injector module confirmatory flow sensor 108(b) or flow sensor 176 at step 610. If the flow information injector module delivery flow sensor 108(a) or flow sensor 174 does not match the flow information from injector module confirmatory flow sensor 108(b) or flow sensor 176, then step 612 provides that the user may be instructed to replace the injector module 107. Furthermore, if during therapy, in one or more embodiments the device may use confirmatory flow sensor 108(b) for flow control and/or delivery. If the flow information from injector module delivery flow sensor 108(a) or flow sensor 174 matches the flow information from injector module confirmatory flow sensor 108(b) or flow sensor 176, then step 614 provides that the user may be instructed to service (e.g. calibrate, replace) gas sensor 182 and/or the device may display the ratio-metric calculated concentrations to the user.

If the calculated ratio-metric concentration matches the NO concentration measured by gas sensor 182, then the primary delivery subsystem controller 144 and/or secondary gas delivery subsystem controller 164 compares the NO concentration measured by gas sensor 182 to the ratio-metric concentration calculated using the therapeutic gas flow reported by the confirmatory sensor 148(a), 148(b), 168, the NO concentration in the gas cylinder, and the breathing gas flow reported by injector module confirmatory flow sensor 108(b) or flow sensor 176 at step 616.

If the calculated ratio-metric concentration for the confirmatory sensors does not match the NO concentration measured by gas sensor 182, then the flow information from delivery flow sensor 146(a), 146(b), 166 is compared to the flow information from its respective confirmatory sensor 148(a), 148(b), 168 at step 606. If the flow information from delivery flow sensor 146(a), 146(b), 166 does not match the flow information from confirmatory sensor 148(a), 148(b), 168, then step 608 provides that the user may be instructed to service the components in fluid communication with the flow control channel being verified, which includes the delivery flow sensor 146(a), 146(b), 166, the respective confirmatory sensor 148(a), 148(b), 168 and/or respective the control valve 143(a), 143(b), 163. Furthermore, if during therapy, the device may fail over to an alternate flow control channel or secondary delivery subsystem. If the flow information from delivery flow sensor 146(a), 146(b), 166 matches the flow information from confirmatory sensor 148(a), 148(b), 168, then the flow information from injector module delivery flow sensor 108(a) or flow sensor 174 is compared to the flow information from injector module confirmatory flow sensor 108(b) or flow sensor 176 at step 610. If the flow information from injector module delivery flow sensor 108(a) or flow sensor 174 does not match the flow information from injector module confirmatory flow sensor 108(b) or flow sensor 176, then step 612 provides that the user may be instructed to replace the injector module 107. Furthermore, if during therapy, in one or more embodiments the device may use confirmatory flow sensor 108(b) for flow control and/or delivery. If the flow information from injector module delivery flow sensor 108(a) or flow sensor 174 matches the flow information from injector module confirmatory flow sensor 108(b) or flow sensor 176, then step 614 provides that the user may be instructed to service (e.g. calibrate, replace) gas sensor 182 and/or the device may display the ratio-metric calculated concentrations to the user.

If the calculated ratio-metric concentration for the confirmatory sensors matches the NO concentration measured by gas sensor 182, then the components in fluid communication with the flow control channel are successfully verified as provided at step 618. The components in fluid communication with the other flow control channels may then be verified by starting at step 602. Once all relevant components have been verified, then the performance verification may proceed further as provided by FIGS. 4A-4C.

Referring back to FIGS. 4A-4C, at step 448, if any and/or all performance verification processes, at step 446, were not successful then therapeutic gas delivery system 100 may proceed to failure processes, at step 406. If successful then performance verification processes may verify three-way 171, at step 450.

Therapeutic gas delivery system 100 may verify flow regulating valve 170, which may be a three-way valve or a directional valve by actuating it such that an initial flow rate (e.g., zero flow) is delivered to the injector module; actuating the flow regulating valve 170 so another set flow rate (e.g. 1 SLPM) is delivered to the injector module; detecting the change seen at the injector module using injector module delivery flow sensor 108(a) and/or injector module confirmatory flow sensor 108(b); and/or then actuating flow regulating valve 170 such that the initial flow rate (e.g., zero flow) returns.

By way of example, therapeutic gas delivery system 100 may verify the flow regulating valve 170 by actuating a three way valve to deliver low pressure air/O₂ at an initial flow rate (e.g., wall flow 10 SLPM); then actuating the flow regulating valve 170 to deliver therapeutic gas through secondary gas delivery subsystem 160, via flow control channel 161, at secondary delivery flow rate (e.g. 1 SLPM); detecting the incremental change seen at the injector module using injector module delivery flow sensor 108(a) and/or injector module confirmatory flow sensor 108(b) (e.g., flow increase of about 10%); and/or then actuating flow regulating valve 170 such that the therapeutic gas flow, NO, flows to 111, and is delivered downstream to the injector module flow sensors and the initial flow rate (wall flow 10 SLPM) returns (e.g., as the incremental NO flow is no longer measured by the injector module flow sensors). In at least some instances, during verification of flow regulating valve 170, the gas analyzer may be exposed to room air, for example, to prevent over-saturation of NO sensor (e.g., from 4880 ppm high concentration).

At step 452, if any and/or all performance verification processes, at step 450, were not successful then therapeutic gas delivery system 100 may proceed to failure processes, at step 406. If successful then performance verification processes prompt the user to connect the injector module and/or sample T to the patient breathing circuit (e.g., as depicted in FIGS. 1-3) and/or connect the external manual ventilation device (e.g., bag valve mask) to outlet 170, at step 454.

At step 456, therapeutic gas delivery system 100 may verify injector module 107 is facing the correct direction and/or in the correct position in the breathing circuit.

In exemplary embodiments, therapeutic gas delivery system 100 may determine when injector module 107 has been inserted into a breathing circuit 209 backwards. This may be accomplished because, amongst other things, the injector module delivery flow sensor and/or the injector module confirmatory flow sensor may be bi-directional flow sensors configured to determine the direction of gas flow through the injector module 107. In various embodiments, the injector module delivery flow sensor and the injector module confirmatory flow sensor are arranged in located relative to each other in series, parallel, skewed, and/or any other configuration.

In various embodiments, the system may guide a user through system setup at the bedside (e.g., at the bedside of a patient and/or intended patient, etc.), which may comprise providing instructions on secondary delivery subsystem connections (e.g., attachment of a valve-mask assembly), injector module 107 connections into the breathing circuit and verify the correct orientation, humidity/temp levels, etc., and on sample T placement in the breathing circuit. In various embodiments, the bi-directional flow sensors in the injector module may indicate gas flow direction and verify the correct orientation to the user.

If injector module 197 is oriented such that it is not facing the correct direction, system 100 may prompt the user to re-position injector module 107 such that it is facing the correct direction.

At step 458, if the injector module is positioned properly in the breathing circuit, therapeutic gas delivery system 100 may then be ready for use (e.g., ready to delivery therapeutic gas to a patient).

In exemplary embodiments, at any time during use of therapeutic gas delivery system 100, the gas analyzer and/or system 100 may initiate a low calibration, at step 470, as described above. For ease, step 470 is shown as occurring after step 404. This is merely for ease and is in no way meant to be a limitation. At step 472, if the low calibration is not successful then therapeutic gas delivery system 100 may retry the low calibration and/or proceed to failure processes, at step 406. If the low calibration is successful it then the sensor is calibrated and may be used during delivery of therapeutic gas to a patient and/or during any relevant steps in the pre-use verification processes (e.g., step 442, etc.).

In exemplary embodiments, at any time during use of therapeutic gas delivery system 100, system 100 may initiate a manifold leak test, at step 480, as described above. At step 482, if the manifold leak test is not successful then therapeutic gas delivery system 100 may proceed to failure processes, at step 406. If the manifold leak test is successful it then the manifold may be used during delivery of therapeutic gas to a patient and/or during any relevant steps in the pre-use verification processes (e.g., step 454, etc.).

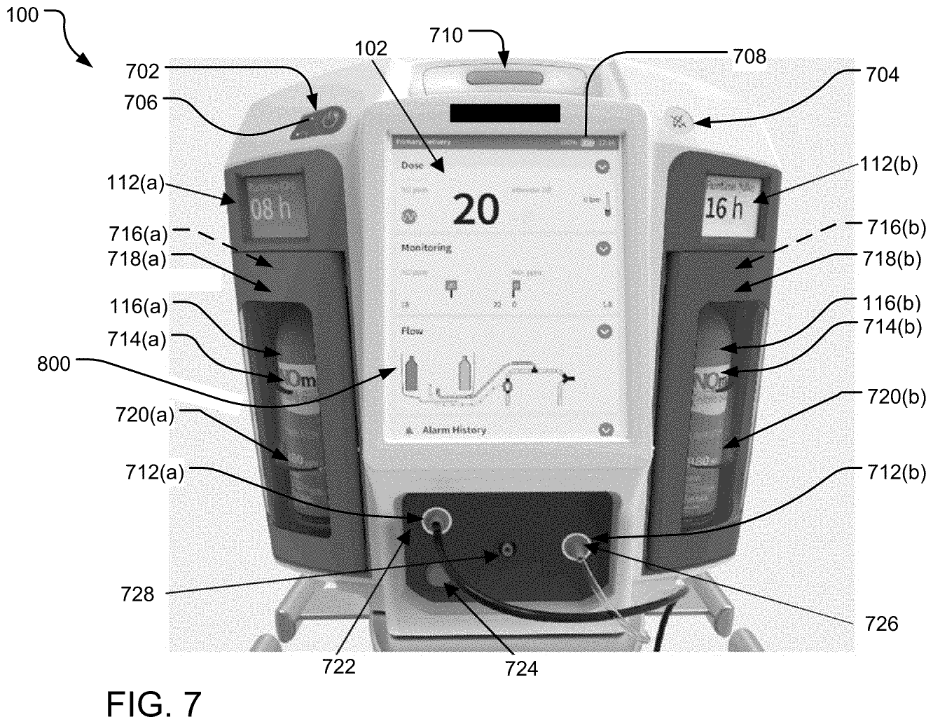

FIG. 7 illustrates one example of a therapeutic gas delivery system 100. The therapeutic gas delivery system 100 may deliver therapeutic gas such as, for example, nitric oxide for inhalation.

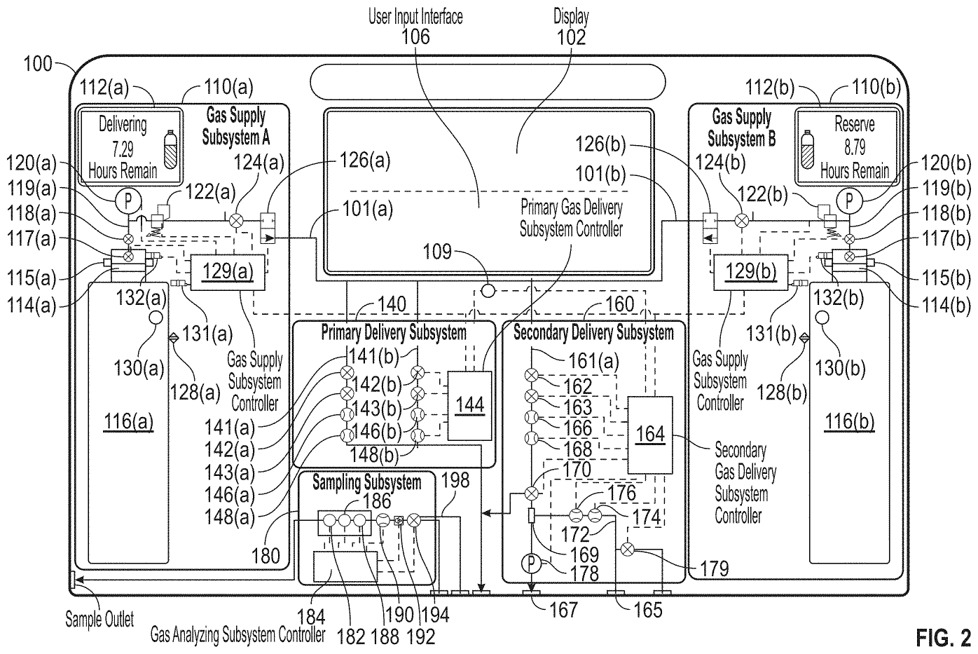

The therapeutic gas delivery system 100 illustrated in FIG. 7 may include one or more same or similar elements as the therapeutic gas delivery system 100 illustrated in FIG. 1, FIG. 2, and/or FIG. 5. For example, the therapeutic gas delivery system 100 may include a central display 102 (e.g., main display), a display 112(a) (e.g., “A” satellite display, “left” satellite display), and/or a display 112(b) (e.g., “B” satellite display, “right” satellite display). In some examples, the central display 102, the display 112(a), and/or the display 112(b) may be a user interface. For example, the central display 102, the display 112(a), and/or the display 112(b) may be a Graphical User Interface (GUI). The GUI (e.g., central display 102, display 112(a), and/or display 112(b)) may include a touchscreen GUI. For example, the central display 102 may be a touchscreen interface with which the user interacts with the therapeutic gas delivery system 100. In at least one example, the central display 102 may be a liquid-crystal display (LCD).

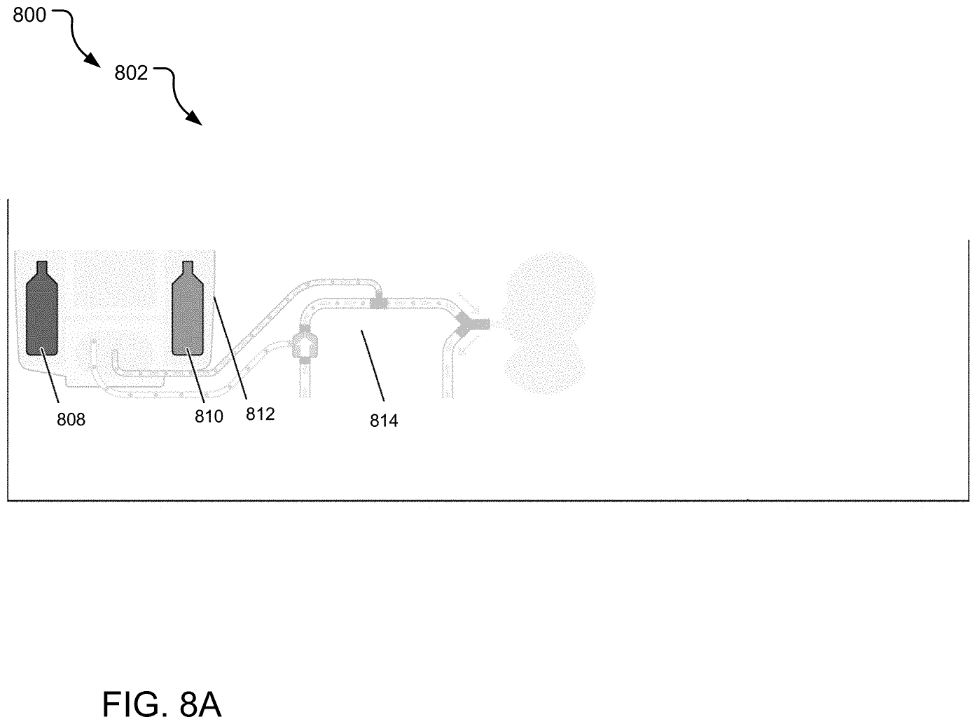

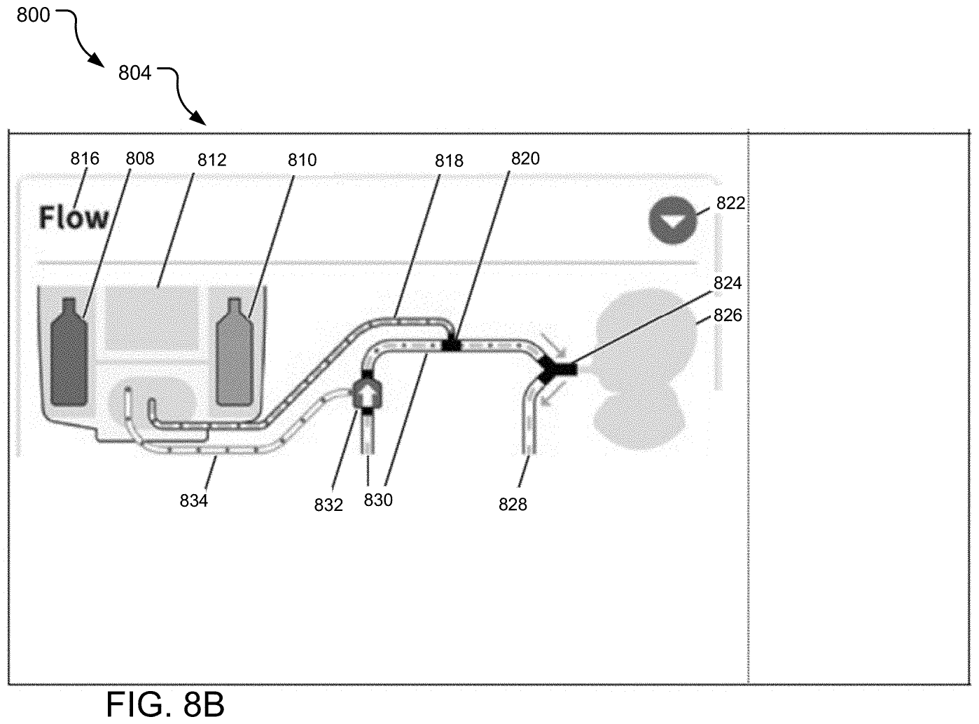

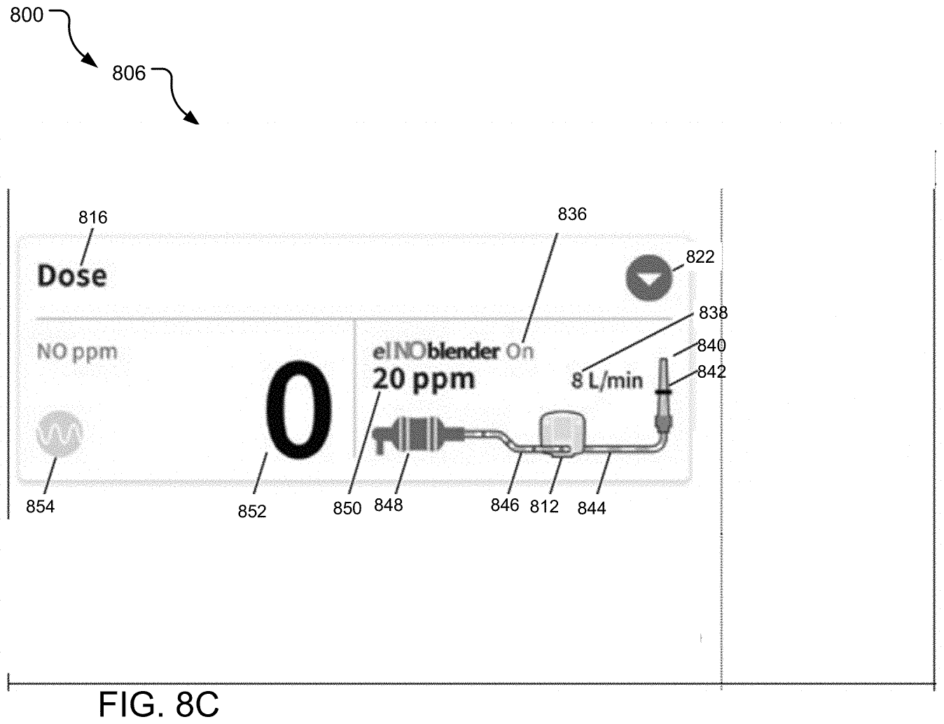

The central display 102, display 112(a), and/or display 112(b) of the therapeutic gas delivery system 100 may provide, or otherwise display, a visual representation of one or more flow parameters (e.g., flow status) and/or system parameters (e.g., cylinder status). In some aspects, the central display 102 may display a graphical, illustrative, or numerical indicator of one or more flow or system parameters. In some embodiments, as illustrated for example in FIGS. 8A-8C, the central display 102 may display one or more display graphics 800 (e.g., main display graphics, tiles). For example, the central display 102 may display a cylinder status graphic 802 (as illustrated in FIG. 8A), a flow tile 804 (as illustrated in FIG. 8B), a dose tile 806 (as illustrated in FIG. 8C), a monitoring tile, an alarm history tile, a treatment history tile, a patient profile tile, a system maintenance tile, a system settings tile, and/or a help tile. FIGS. 8A-8C are discussed in further detail below.

Continuing with FIG. 7, the therapeutic gas delivery system 100 may include one or more user interface buttons, such as an on/shutdown button 702 and/or an alarm silence button 704. In some examples, an on/shutdown button 702 and an alarm silence button 704 may both be located on the front-side of the therapeutic gas delivery system 100. In some examples, an on/shutdown button 702 and an alarm silence button 704 may both be located near the top side of the therapeutic gas delivery system 100.

The on/shutdown button 702 may select a state of powered on or a state of shutdown/off for the therapeutic gas delivery system 100. For example, when the therapeutic gas delivery system 100 is shutdown, selecting or pressing the on/shutdown button 702 may turn the therapeutic gas delivery system 100 on. When the therapeutic gas delivery system 100 is turned on, selecting the on/shutdown button 702 may shutdown or turn off the therapeutic gas delivery system 100.

The on/shutdown button 702 may include a visual indicator 706 that indicates the status of the electric power supply and/or the status of the internal battery of the therapeutic gas delivery system 100. For example, the visual indicator 706 may be an LED light. For example, the visual indicator 706 may be illuminated, unilluminated, and/or flashing to visually indicate a corresponding power supply status and/or a corresponding battery charge status. In an example, the visual indicator 706 may be illuminated when the power supply is connected and/or the internal battery is charging or fully charged. In another example, the visual indicator 706 may be unilluminated when the power supply is disconnected and/or the battery is not charging and/or dead. The visual indicator may be flashing when the battery is charging. In some examples, when it is illuminated, the visual indicator 706 may display a specific color to indicate a specific power supply status and/or battery charge status. In some examples, one or more colors may be constantly displayed. In other examples, one or more colors may be flashing.

In some examples, when the therapeutic gas delivery system 100 is shut down and the therapeutic gas delivery system 100 is not connected to an electric power supply, the visual indicator 706 may be unilluminated to indicate that the internal battery is not charging.

In some examples, when the therapeutic gas delivery system 100 is shut down but the therapeutic gas delivery system 100 is connected to an electric power supply, the visual indicator 706 may be illuminated. For example, the visual indicator 706 may display a constant first color light to indicate that the internal battery is charging or a constant second color light to indicate that the internal battery is not charging. In an example, the first color may be amber and the second color may be green.

In some examples, when the therapeutic gas delivery system 100 is powered on, the central display 102 may display the power supply status and/or battery charge status. For example, a status bar 708 on the central display 102 may display the power supply status and/or battery charge status. In some examples, the visual indicator 706 may be unilluminated when the status bar 708 displays the power supply status and/or battery charge status. In some aspects, the status bar 708 may include an illustrative indicator to indicate that the therapeutic gas delivery system 100 is connected to an electric power supply. In one example, the illustrative indicator may be a lightning bolt. In some aspects, the status bar 708 may include a graphical representation and/or a numerical representation to indicate battery power remaining, battery level indicator, and/or a charge status. In some examples, the graphical representation may be a bar graph displaying a percentage of the battery power remaining, battery level indicator, and/or charge status. In another example, the numerical representation may be a number indicating a percentage of the battery power remaining, battery level indicator, and/or charge status.

The alarm silence button 704 may silence one or more alarms of the therapeutic gas delivery system 100. The alarms may be audible and/or visual alarms. For example, when an alarm activates on the therapeutic gas delivery system 100, selecting the alarm silence button 704 may silence the alarm for a pre-determined period of time. For example, the alarm silence button 704 may be selected by pressing or pushing the alarm silence button 704. In some examples, the pre-determined period of time may be approximately one minute, approximately two minutes, approximately three minutes, approximately four minutes, or approximately five minutes. In at least one example, selecting the alarm silence button 704 may silence an alarm for approximately two minutes. In some examples, the therapeutic gas delivery system 100 may be configured so that one or more alarms may not be silenced by selecting the selecting the alarm silence button 704. For example, the therapeutic gas delivery system 100 may be configured so that a secondary alarm for a device power failure or a main alarm speaker failure may not be silenced by the alarm silence button 704. In at least one example, selecting the alarm silence button 704 may silence some alarms but not other alarms. For example, selecting the alarm silence button 704 may silence low priority alarms, but not high priority alarms such as a secondary alarm for a device power failure.

The alarm silence button 704 may include a visual indicator that indicates the status of one or more alarms of the therapeutic gas delivery system 100. For example, the visual indicator for the alarm silence button may be an LED light. For example, the visual indicator may be illuminated, unilluminated, and/or flashing to visually indicate a corresponding alarm status when an alarm occurs in the therapeutic gas delivery system 100. For example, the alarm status may be no alarm, high priority alarm, or low priority alarm. In some examples, when it is illuminated, the visual indicator may display a specific color (e.g., red, yellow) to indicate a specific alarm status. In some examples, one or more colors may be constantly displayed. In other examples, one or more colors may be flashing. For example, the visual indicator of the alarm silence button 704 may be unilluminated to indicate that there is no active alarm in the therapeutic gas delivery system 100. As another example, the visual indicator may flash a first color to indicate a high priority alarm. As another example, the visual indicator may display a constant second color to indicate a low priority alarm. In an example, the first color may be red, and the second color may be yellow.

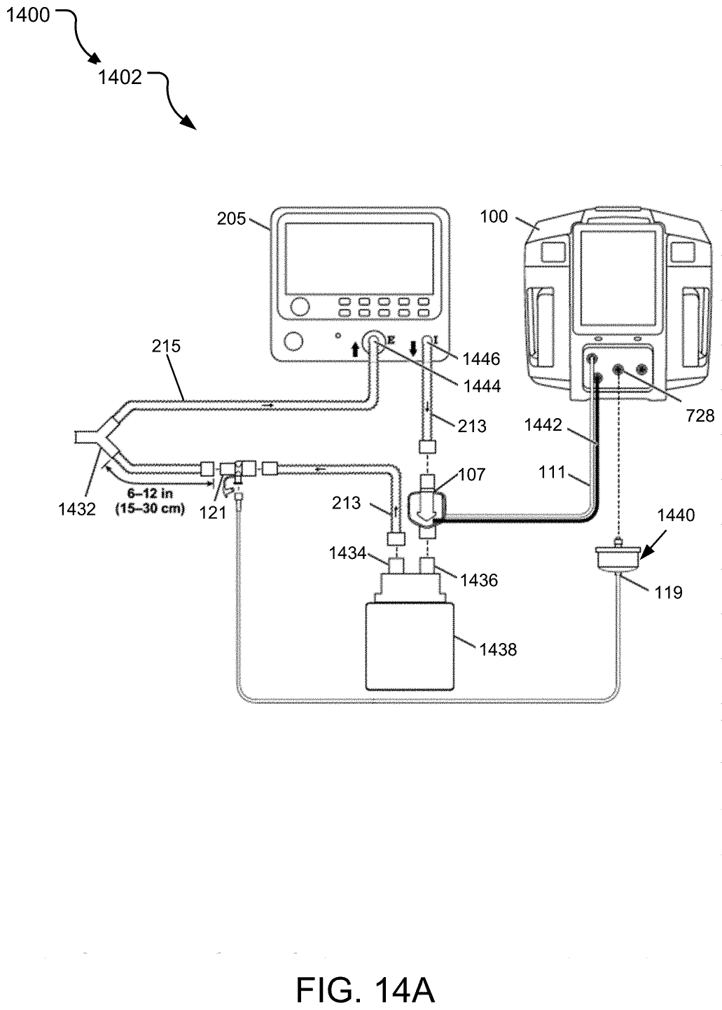

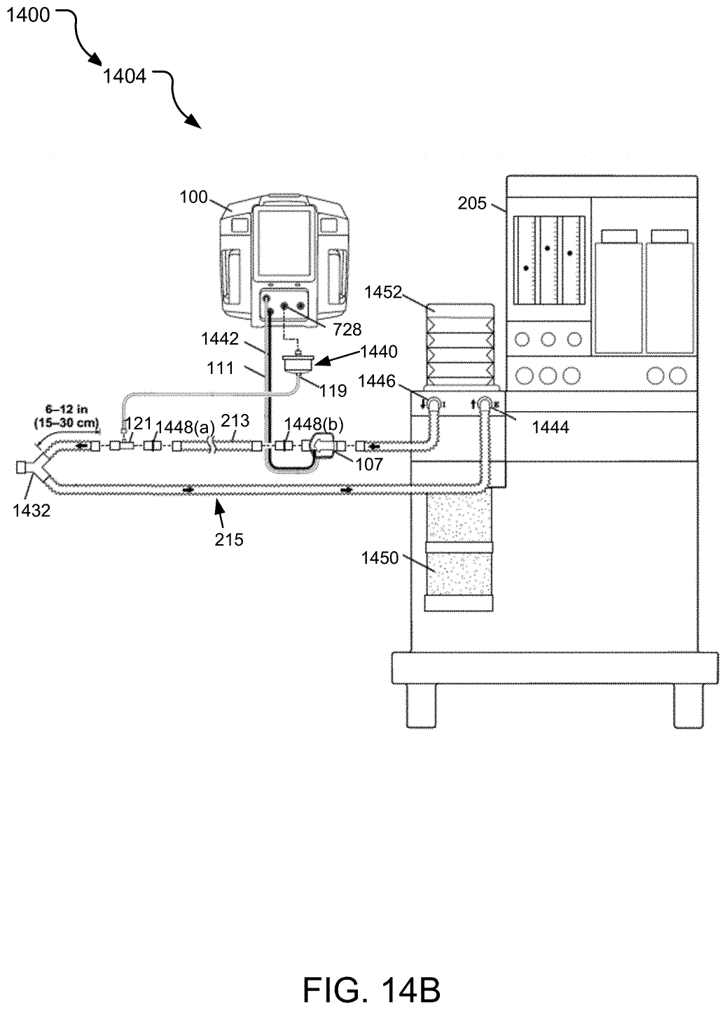

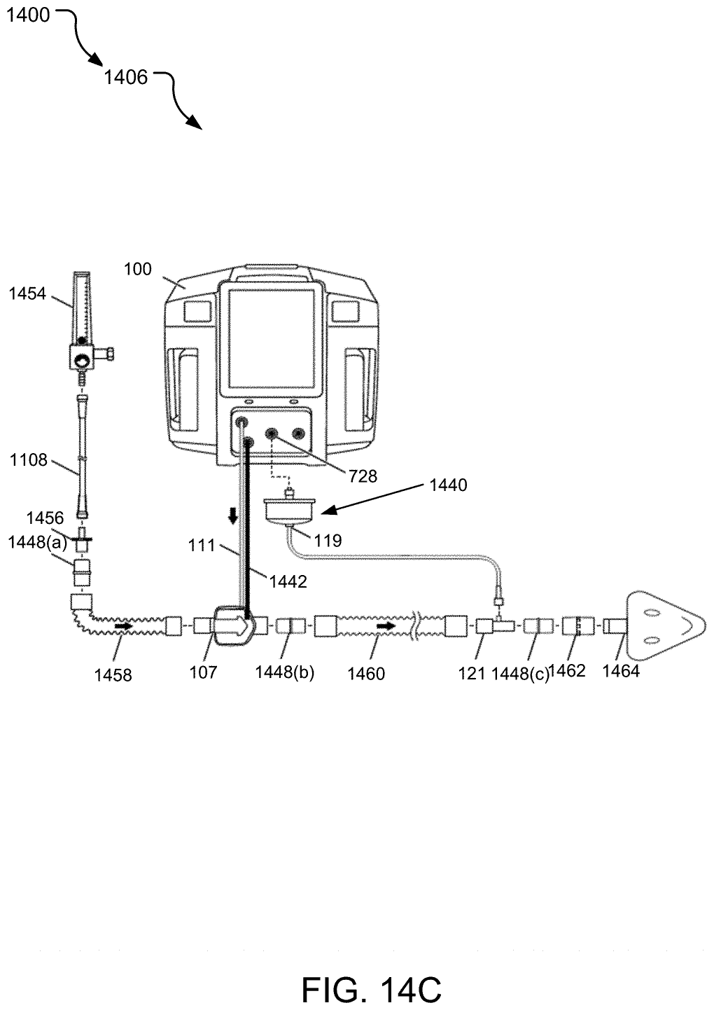

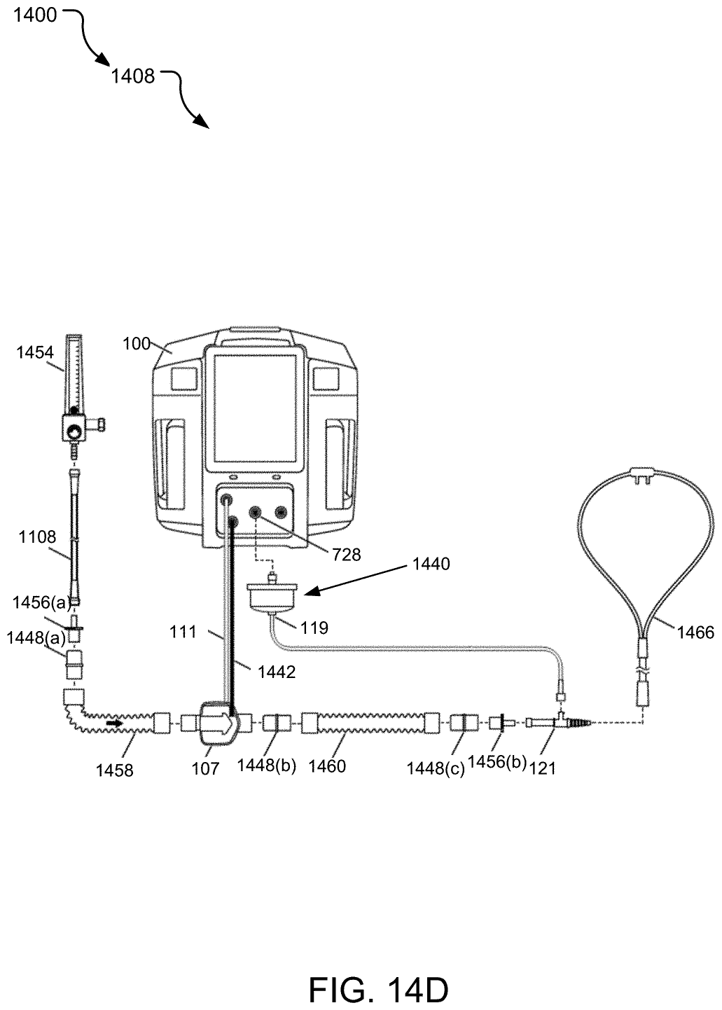

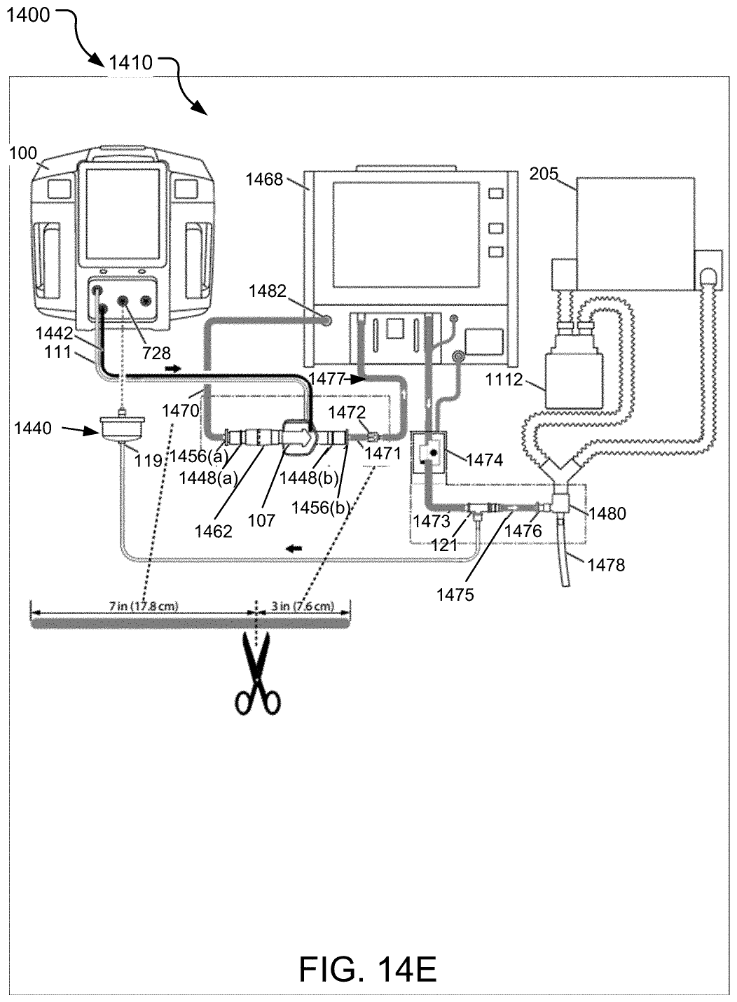

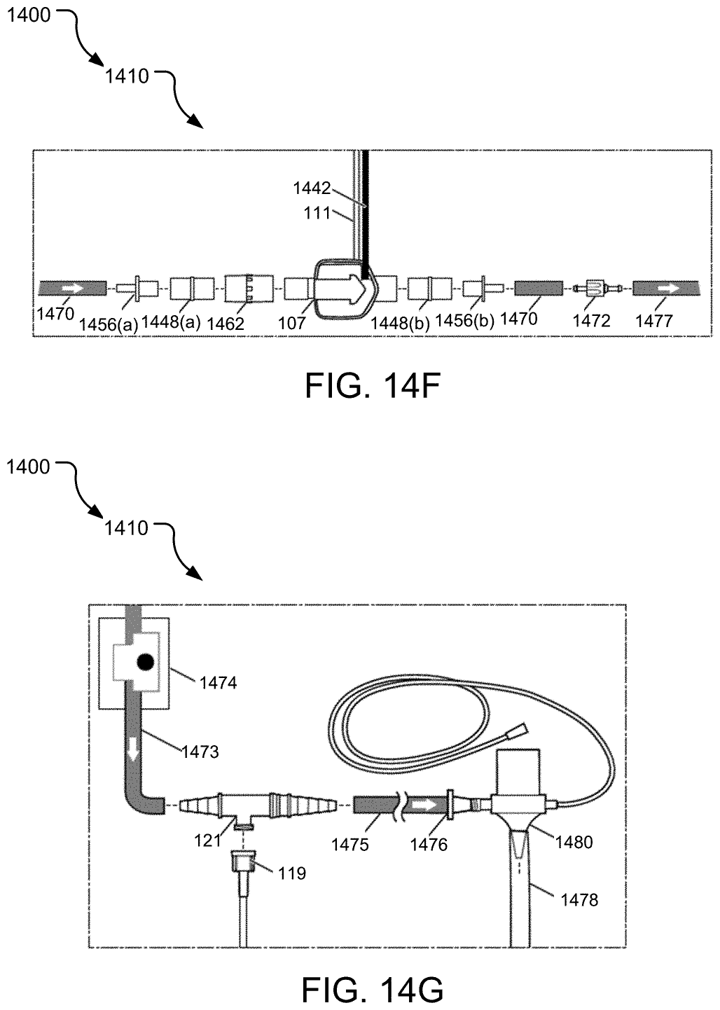

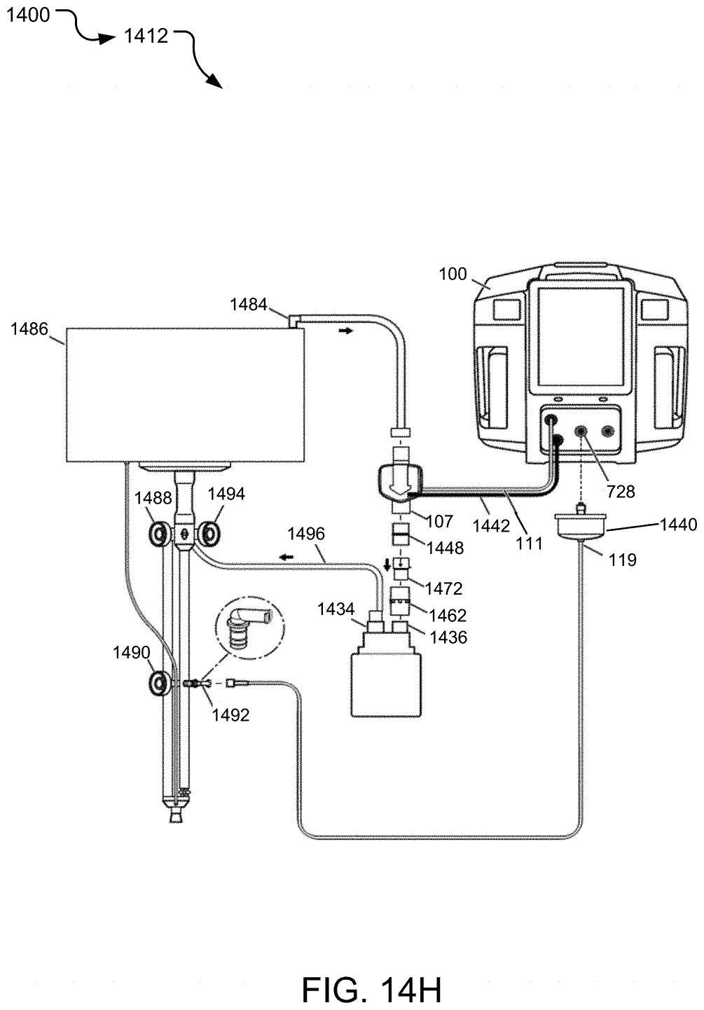

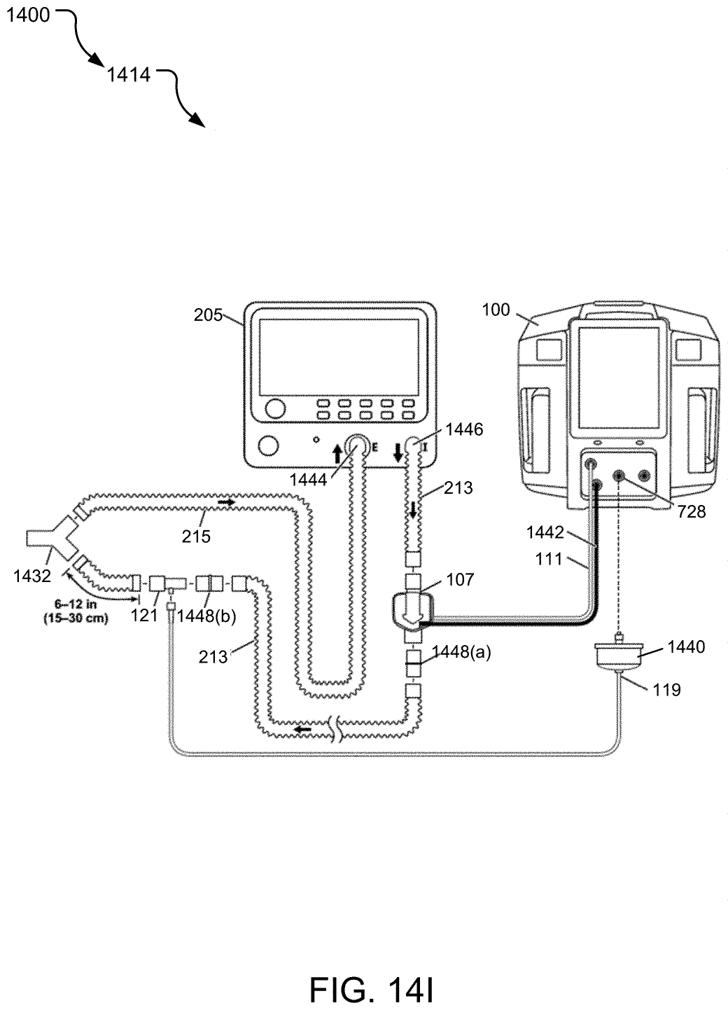

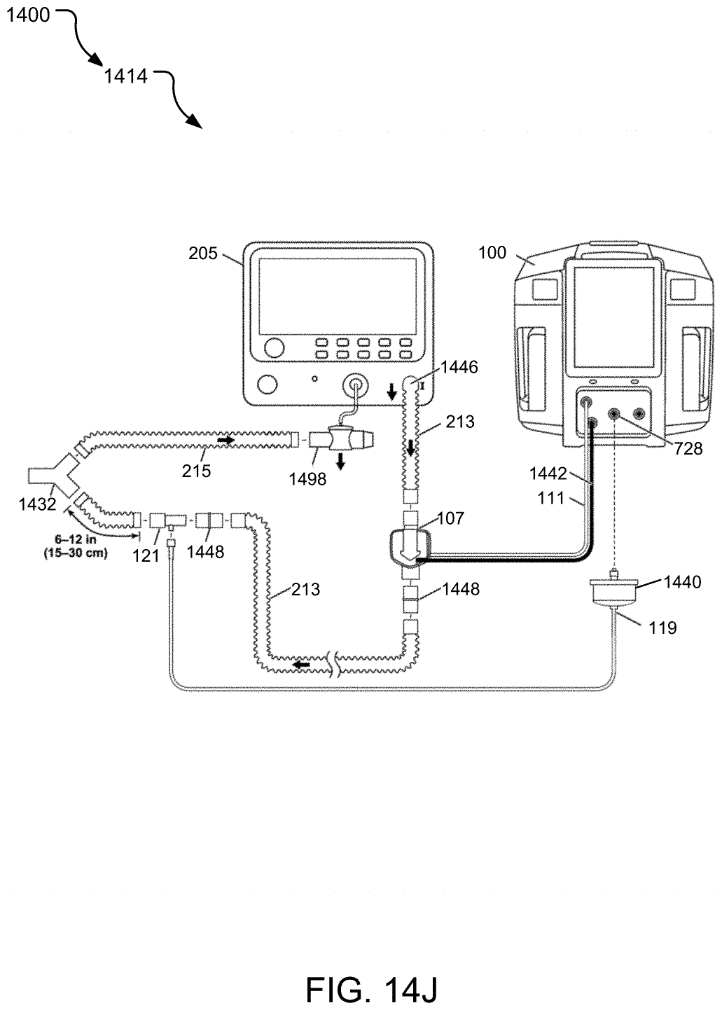

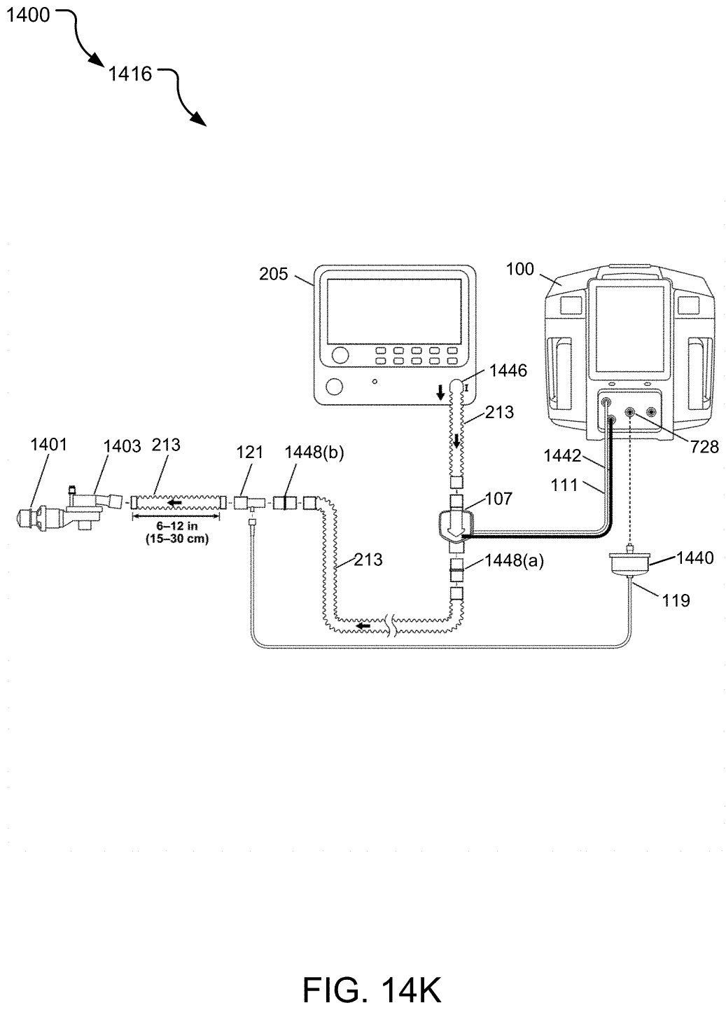

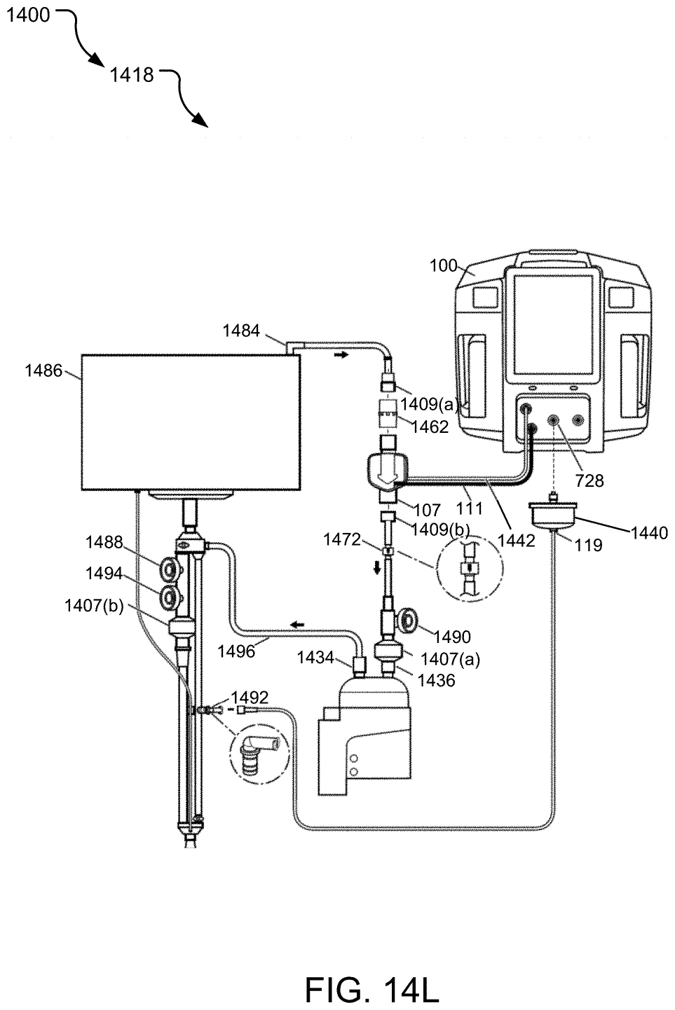

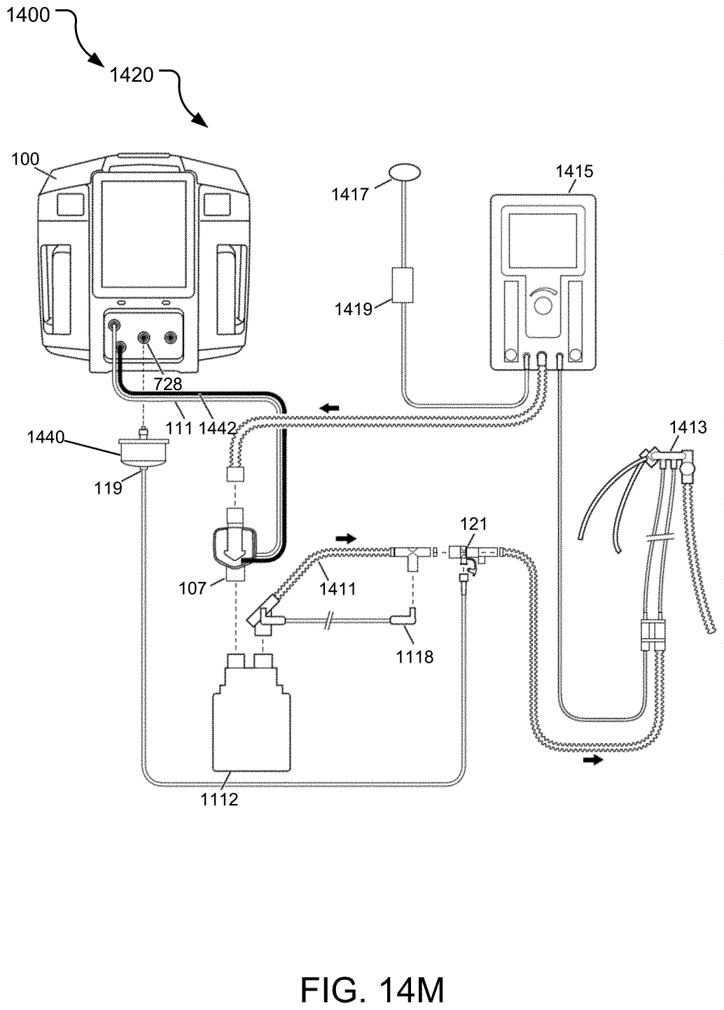

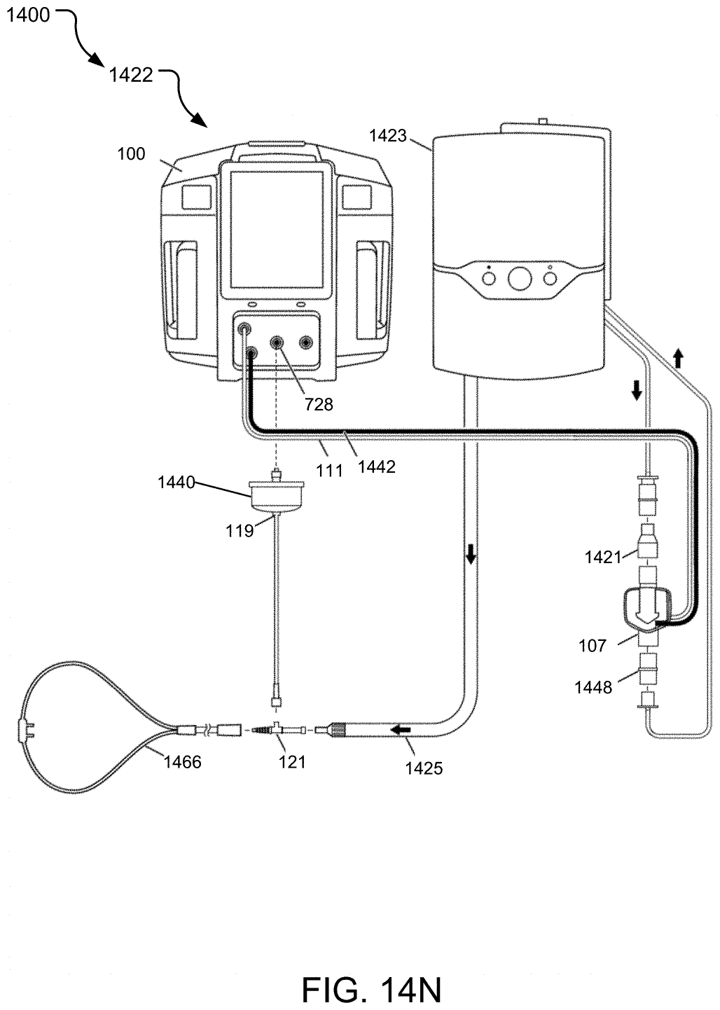

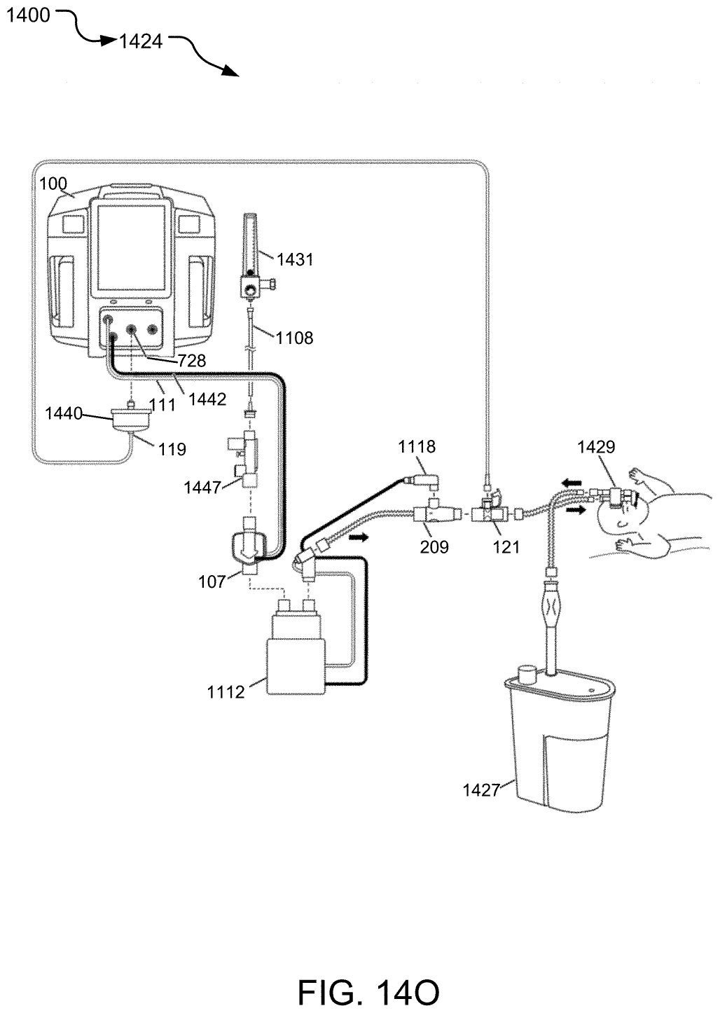

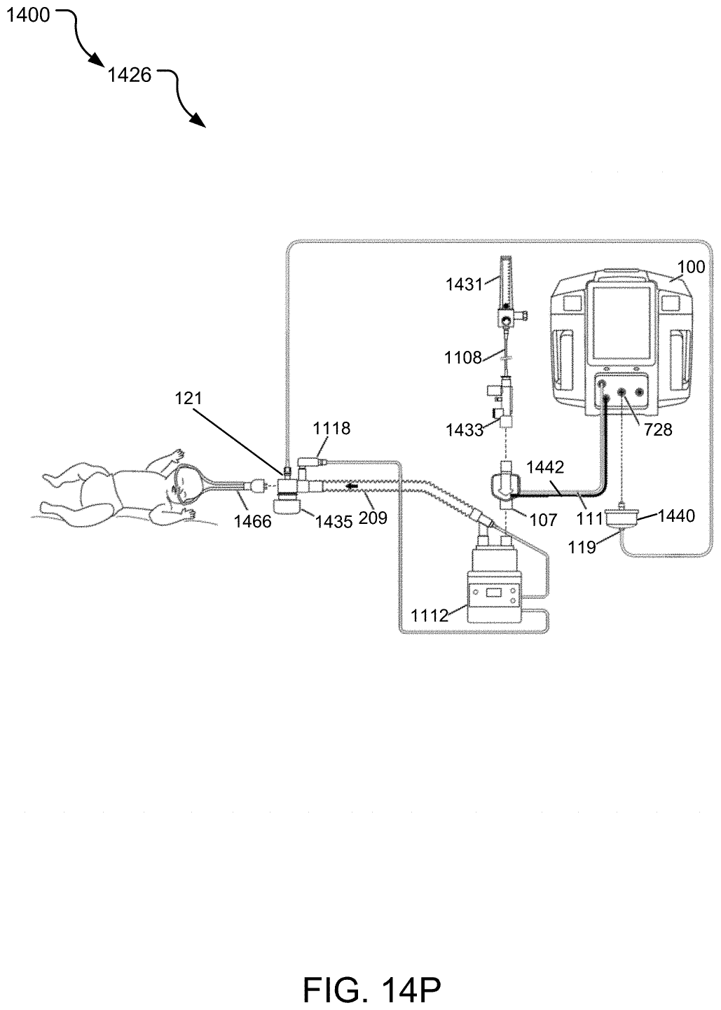

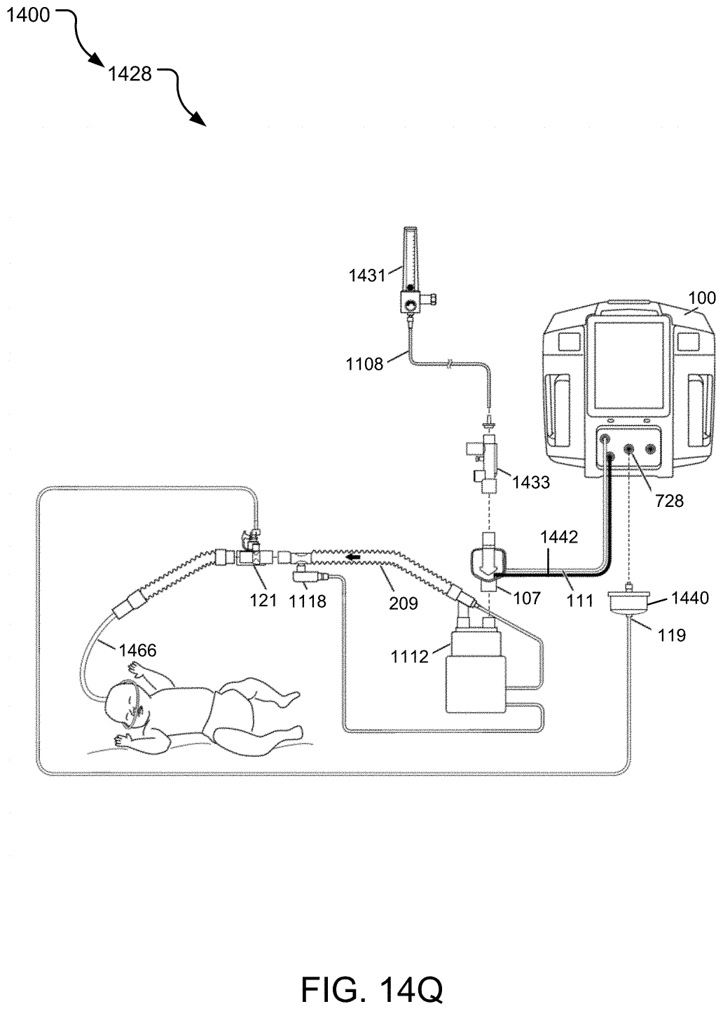

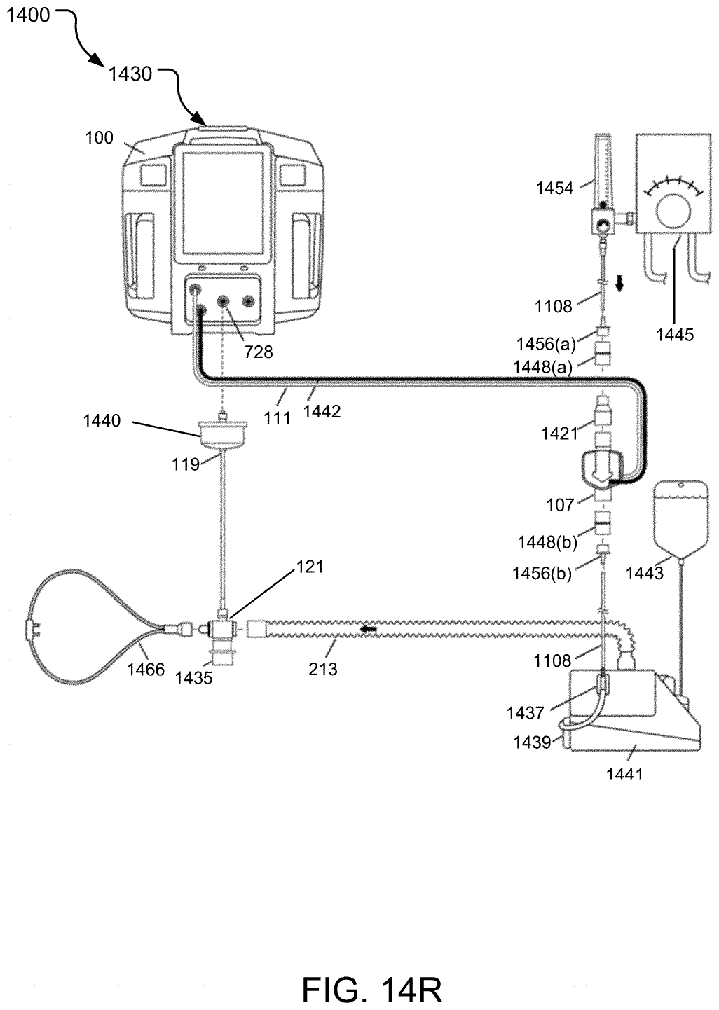

Continuing with FIG. 7, the therapeutic gas delivery system 100 may include an injector module outlet 722, an injector module cable socket 724 for electrical connection of the injector module to a power source, a secondary delivery subsystem outlet 726, and/or a sample line inlet 728 to connect to a patient gas sample line. In some examples, the injector module outlet 722, injector module cable socket 724, secondary delivery subsystem outlet 726, and/or sample line inlet 728 may be located on the front side of the therapeutic gas delivery system 100. These components are discussed in FIGS. 14A-14R, which are examples of breathing circuits 1400 that include the therapeutic gas delivery system 100.

Continuing with FIG. 7, the therapeutic gas delivery system 100 (e.g., EVOLVE delivery device) may include a global status indicator 710 and/or one or more port indicators 712 (e.g., 712(a), 712(b)). In some examples, the global status indicator 710 and/or one or more port indicators 712 may be located on the front-side of the therapeutic gas delivery system 100. The indicators may provide for a convenient way of displaying a status to a user in a quick and efficient manner.

The global status indicator 710 may include a visual indicator (e.g., an LED light) that indicates the global status (or statuses) of the therapeutic gas delivery system 100. For example, the global status indicator 710 may be illuminated, unilluminated, and/or flashing to visually indicate a corresponding global status (e.g., not alarming or delivering, delivering, low priority alarm, high priority alarm). In some examples, the global status indicator 710 may display a color when it is illuminated. In some examples, one or more colors may be constantly displayed. In other examples, one or more colors may be flashing. In some examples, the global status indicator 710 may be located on the handle of the therapeutic gas delivery system 100.

In some examples, the global status indicator 710 may indicate that the status of the therapeutic gas delivery system 100 is not alarming and/or not delivering (e.g., not delivering therapeutic gas). For example, when the therapeutic gas delivery system 100 is not alarming or not delivering, the global status indicator 710 may be unilluminated. In some examples, the global status indicator 710 may indicate that status of the therapeutic gas delivery system 100 is delivering (e.g., delivering therapeutic gas). For example, when the therapeutic gas delivery system 100 is delivering, the global status indicator 710 may display a first color. In some examples, the global status indicator 710 may indicate that the status of the therapeutic gas delivery system 100 is a low priority alarm. For example, when the therapeutic gas delivery system 100 is in a low priority alarm, the global status indicator 710 may display a constant second color. In some examples, the global status indicator 710 may indicate that the status of the therapeutic gas delivery system 100 is a high priority alarm. For example, when the therapeutic gas delivery system 100 is in a high priority alarm, the global status indicator 710 may display a flashing third color For example, the first, second, and third colors may be symbolic of a level of alarm. The level of alarm may also be conveyed when the global status indicator 710 is illuminated, unilluminated, or flashing. In an example, the first color may be teal, the second color may be yellow, and the third color may be red.

The one or more port indicators 712 (e.g., 712(a), 712(b)) may include one or more visual indicators (e.g., LED light), which may indicate the status of one or more corresponding ports (e.g., port status of injector module outlet 722, port status of secondary delivery subsystem outlet 726) of the therapeutic gas delivery system 100. For example, each port indicator 712 may be illuminated, unilluminated, and/or flashing to indicate a port status (e.g., delivering therapeutic gas, not delivering therapeutic gas) of the corresponding port. In some examples, the port indicator 712 may display a color when it is illuminated. In some examples, one or more colors may be constantly displayed. In other examples, one or more colors may be flashing. In some examples, the one or more colors may be indicative of a port status. In an example, the one or more colors may be teal, green, and/or red. In some examples, each port indicator 712 may be located at (e.g., a ring-shape around) the corresponding port of the therapeutic gas delivery system 100.

In some examples, a port indicator 712(a) may be located at the injector module outlet 722 and may indicate whether the injector module outlet 722 is delivering therapeutic gas. In some examples, a port indicator 712(b) may be located at the secondary delivery subsystem outlet 726 and may indicate whether the secondary delivery subsystem outlet 726 is delivering therapeutic gas.

In some examples, a port indicator 712 (e.g., 712(a), 712(b)) may be illuminated to indicate that the corresponding port is delivering therapeutic gas or unilluminated to indicate that the corresponding port is not delivering therapeutic gas. For example, to indicate therapeutic gas delivery though only the injector module outlet 722, port indicator 712(a) may be illuminated and port indicator 712(b) may be unilluminated. To indicate therapeutic gas delivery though only the secondary delivery subsystem outlet 726, port indicator 712(b) may be illuminated and port indicator 712(a) may be unilluminated. To indicate therapeutic gas delivery through both the injector module outlet 722 and the secondary delivery subsystem outlet 726 (e.g., primary delivery and secondary delivery subsystem delivery), both port indicator 712(a) and port indicator 712(b) may be illuminated. To indicate no therapeutic gas delivery, both port indicator 712(a) and port indicator 712(b) may be unilluminated.

In other examples, a port indicator 712 that is unilluminated may indicate that the corresponding port is delivering therapeutic gas, whereas a port indicator 712 that is illuminated may indicate that the corresponding port is not delivering therapeutic gas. In some examples, a port indicator 712 may be illuminated to indicate whether the corresponding port is delivering therapeutic gas. For example, a port indicator 712 may display a color (e.g., teal, green) to indicate that the corresponding port is delivering therapeutic gas. A port indicator 712 may display a different color (e.g., red) to indicate that the corresponding port is not delivering therapeutic gas.

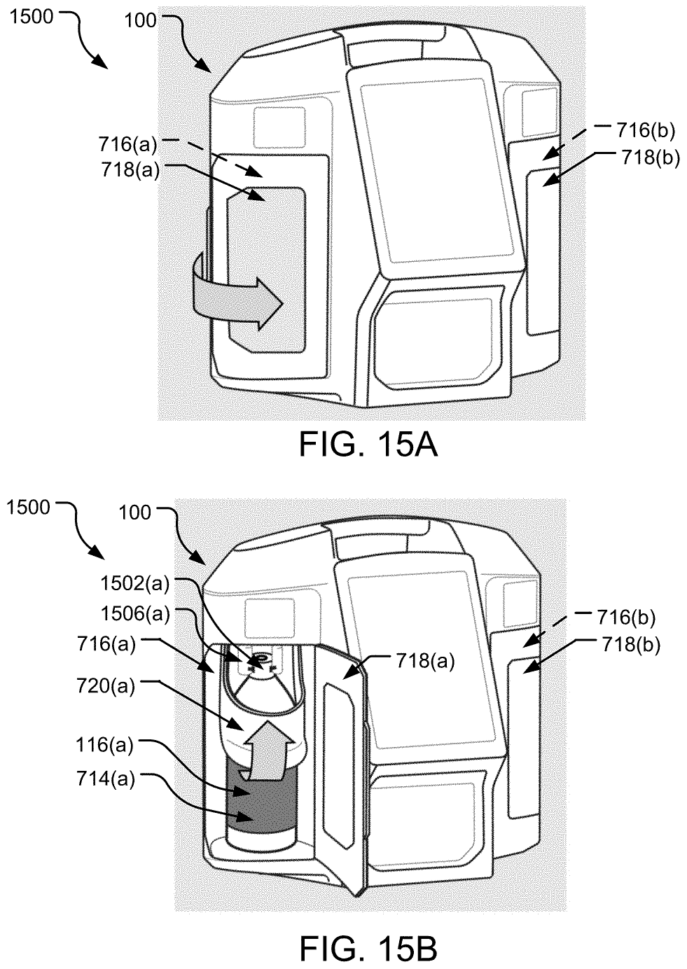

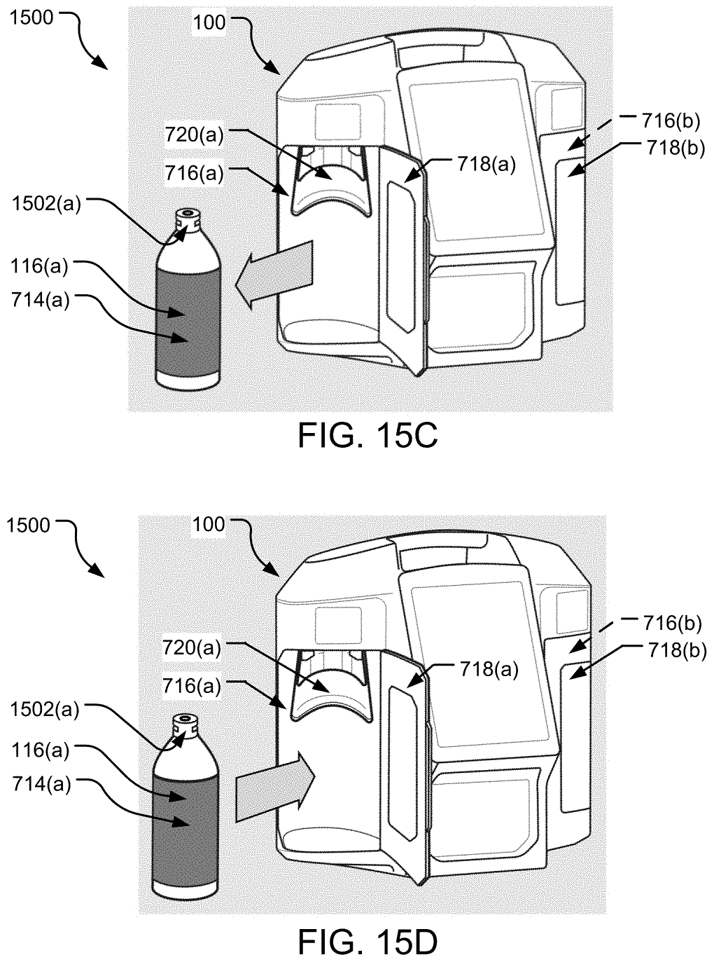

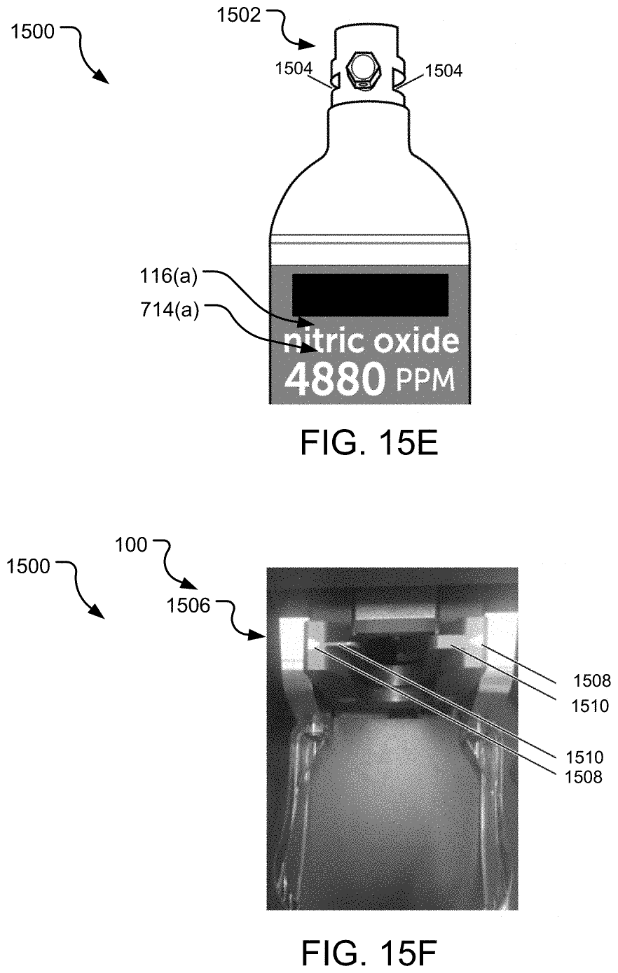

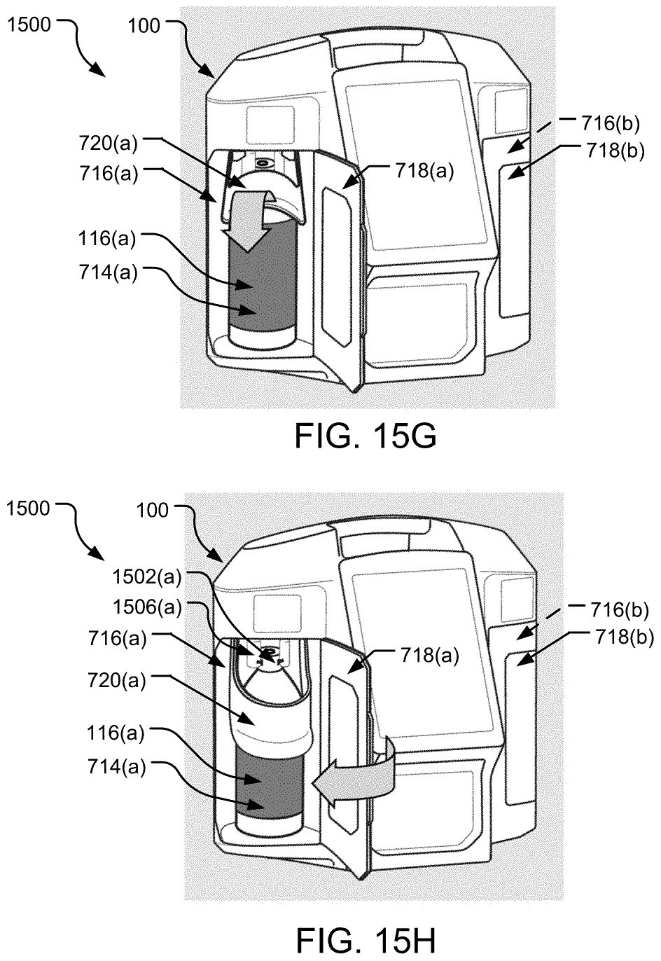

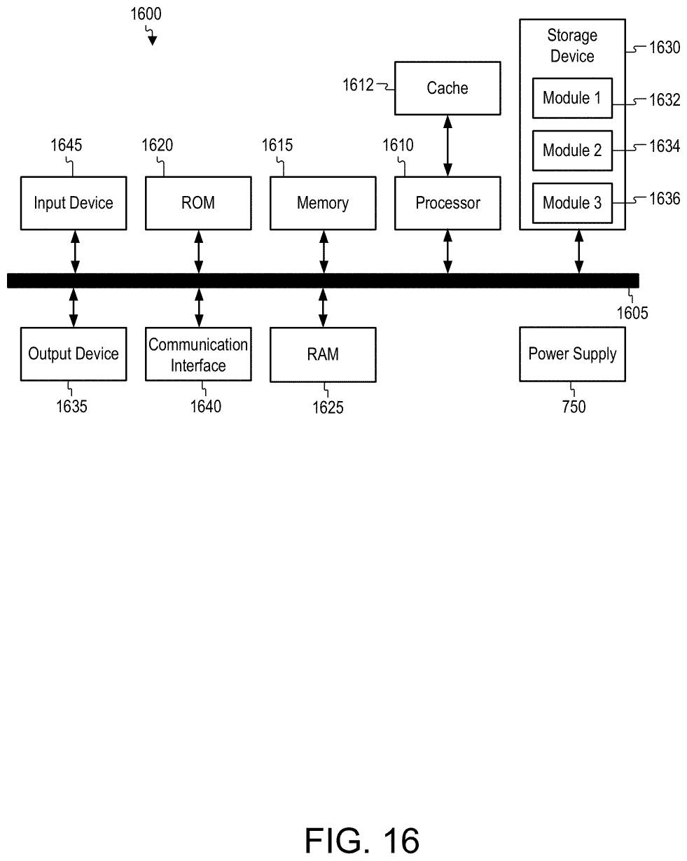

Continuing with FIG. 7, the therapeutic gas delivery system 100 may include one or more therapeutic gas sources 116 (e.g., 116(a), 116(b)), as previously discussed. In some examples, each therapeutic gas source 116(a), 116(b) may be a compressed gas cylinder 714 (e.g., 714(a), 714(b)). Each compressed gas cylinder 714(a), 714(b) may be received within a cylinder bay 716 (e.g., 716(a), 716(b)). A door 718 (e.g., 718(a), 718(b)) of the cylinder bay 716(a), 716(b) may be operable to open and close with respect to each respective bay 716. For example, the door 718 may enclose each bay 716 when the door 718 is closed or allow access to the bay 716 when the door 718 is open. Each compressed gas cylinder 714 (e.g., 714(a), 714(b)) may include a lever 720 (e.g., 720(a), 720(b)) (e.g., a cylinder valve lever). Each compressed gas cylinder 714 may be loaded into and/or replaced from each respective bay 716, as illustrated for example in FIGS. 15A-15H.

FIGS. 8A-8C illustrate examples of display graphics 800. The display graphics 800 may display, for example, system parameters and/or flow parameters. The display graphics 800 may include, for example, a cylinder status graphic 802 (as illustrated in FIG. 8A), a flow tile 804 (as illustrated in FIG. 8B), and/or a dose tile 806 (as illustrated in FIG. 8C). Additional examples of display graphics include a monitoring tile, an alarm history tile, a treatment history tile, a patient profile tile, a system maintenance tile, a system settings tile, and/or a help tile. In some aspects, the central display 102, status display 112(a), and/or status display 112(b) of the therapeutic gas delivery system 100 may display one or more display graphics 800. For example, as previously discussed with respect to FIG. 7, one or more display graphics 800 may be displayed on the central display 102.

FIG. 8A illustrates one example of a cylinder status graphic 802. The cylinder status graphic 802 may include, for example, a left cylinder status 808, a right cylinder status 810, a therapeutic gas delivery system graphic 812, and/or a breathing circuit graphic 81. The left cylinder status 808 may display the status of a left compressed gas cylinder and the right cylinder status 810 may display the status of a right compressed gas cylinder. For example, the left cylinder status 808 (as illustrated in FIG. 8A) may indicate the status of compressed gas cylinder 714(a) (as illustrated in FIG. 7) and the right cylinder status 810 may indicate the status of compressed gas cylinder 714(b).

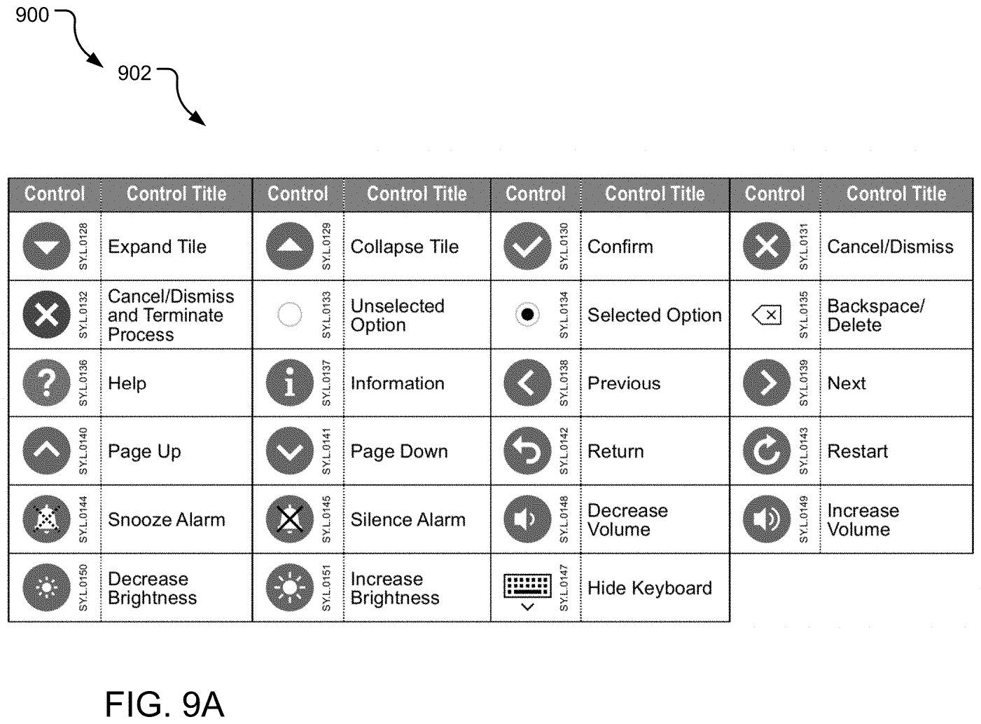

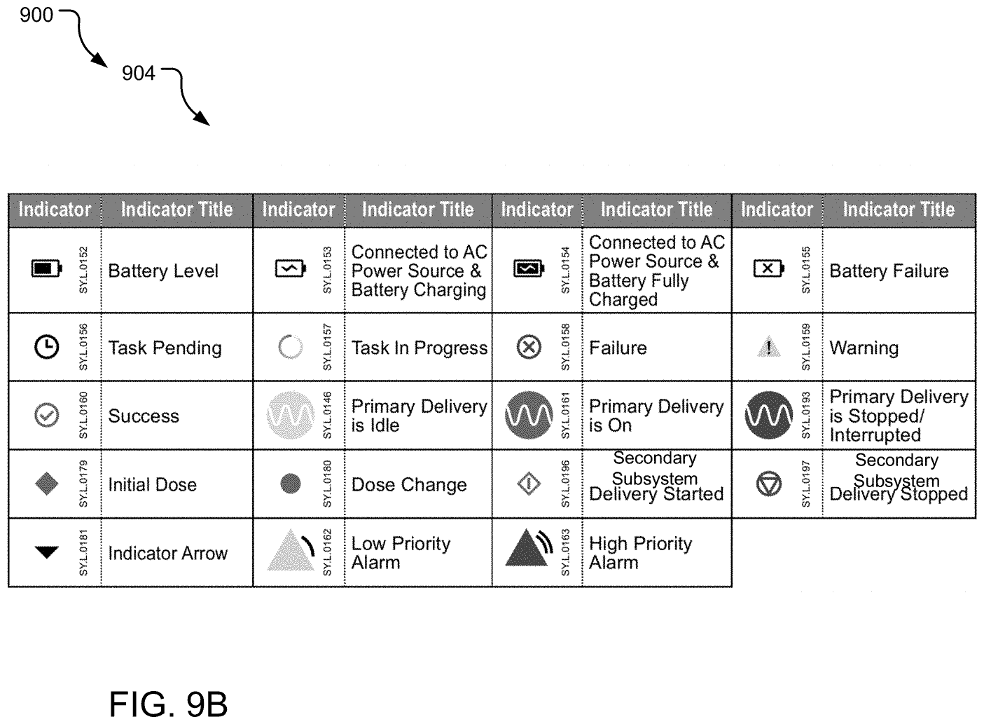

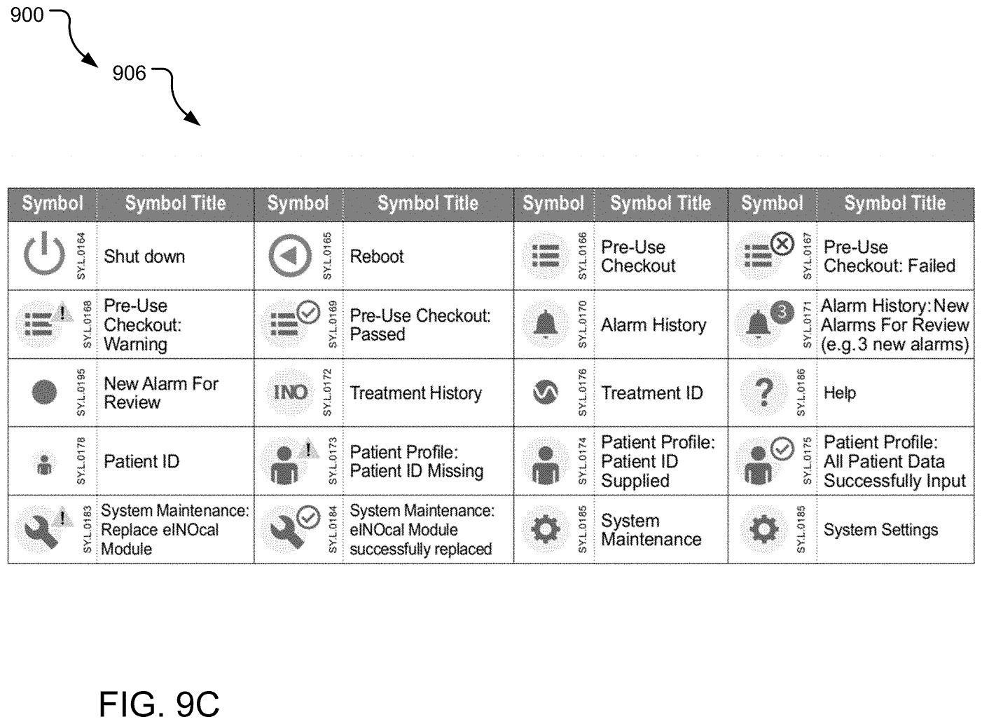

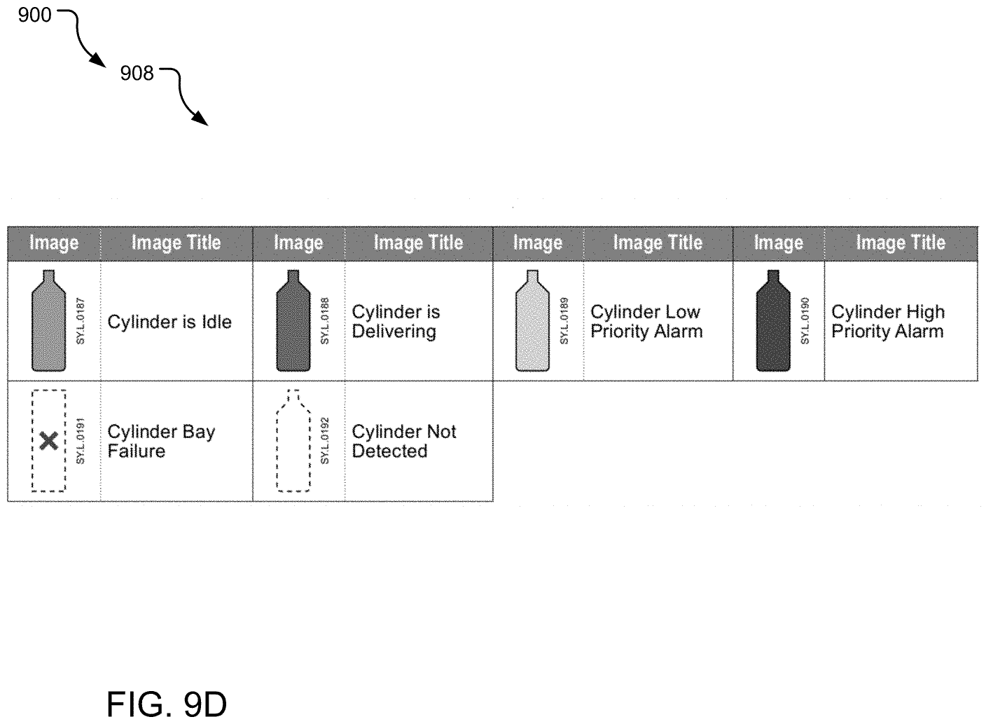

Continuing with FIG. 8A, the right cylinder status 810 and left cylinder status 808 may display a status of the corresponding cylinder. For example, the status of a cylinder may be present, not present, properly functioning, error, and/or not properly functioning. In some aspects, the right cylinder status 810 and/or left cylinder status 808 may illuminate when the corresponding cylinder is functioning properly and may dim (e.g., fade, darken) when the corresponding cylinder is not present or not properly functioning. In some aspects, the left cylinder status 808 and/or right cylinder status 810 may display a corresponding compressed gas cylinder images 908 as illustrated in FIG. 9D and discussed below. In some aspects, the right cylinder status 810 and/or left cylinder status 808 may provide a percentage of available gas in the corresponding cylinder and may display a graphic, such as a graphical representation and/or a numerical representation, that indicates the percentage of available gas and/or amount of gas remaining. For example, the graphical representation may be a bar graph indicating the percentage and/or amount of gas remaining. For example, the numerical representation may be a number indicating the percentage and/or amount of gas remaining.

FIG. 8B illustrates one example of a flow tile 804. The flow tile 804 illustrates a primary delivery circuit flow diagram. The flow tile 804 may include, for example, a tile name 816, a left cylinder status 808, a right cylinder status 810, a therapeutic gas delivery system graphic 812, a patient gas sample line graphic 818, a sample tee graphic 820, an expand tile button 822, a patient wye graphic 824, a patient icon 826, a breathing circuit expiratory limb graphic 828, a breathing circuit inspiratory limb graphic 830, an injector module graphic 832, and/or an delivery conduit graphic 834.

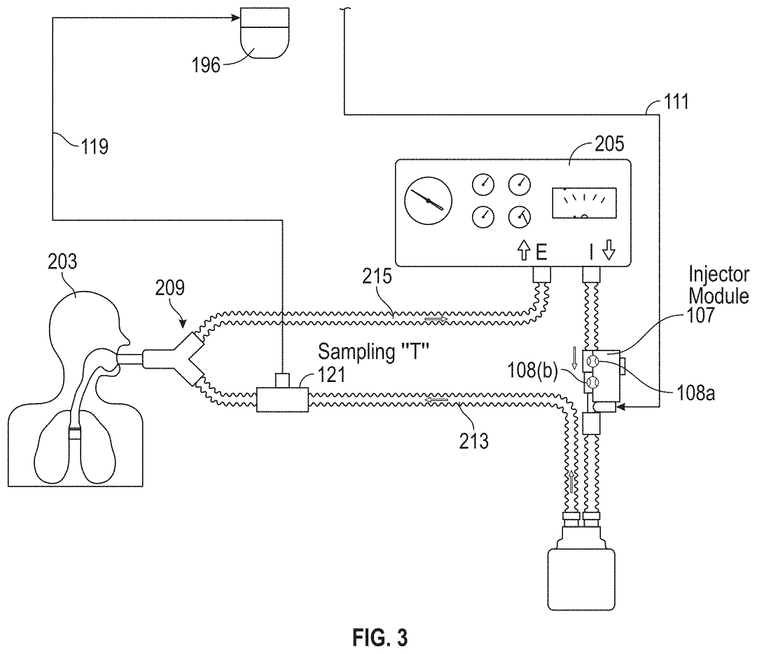

In some aspects, the patient gas sample line graphic 818, the delivery conduit graphic 834, the breathing circuit inspiratory limb graphic 830, the injector module graphic 832, and/or the breathing circuit expiratory limb graphic 828 (as illustrated for example in FIG. 8B) may each indicate the status of the flow with respect to the corresponding component of the patient breathing circuit 209 (as illustrated for example in FIG. 1 and FIG. 3). For example, the status of the flow may be flow, no flow, or alarm. For example, the patient gas sample line graphic 818 may indicate the status of the flow of the sample line 119, the delivery conduit graphic 834 may indicate the status of the flow delivery line and/or conduit 111, the breathing circuit inspiratory limb graphic 830 may indicate status of the flow of the inspiratory limb 213, the injector module graphic 832 may indicate the status of the flow the injector module 107, and/or the breathing circuit expiratory limb graphic 828 may indicate the status of the flow of the expiratory limb 213.

Continuing with FIG. 8B, selecting (e.g., pressing, pushing) the expand tile button 822 may expand the interface to show the full flow tile graphic. The left cylinder status 808 and right cylinder status 810 may display a status of the corresponding cylinders. For example, the status of the corresponding cylinders may be present, not present, properly functioning, error, or not properly functioning. In some aspects, the left cylinder status 808 and/or right cylinder status 810 may illuminate when the corresponding cylinder is functioning properly and/or present. The left cylinder status 808 and right cylinder status 810 may dim (e.g., fade, darken) when the corresponding cylinder is not functioning properly, not present, and/or has an error. The patient gas sample line graphic 818 may display the status of the patient gas sample line. In some aspects, the patient gas sample line graphic 818 may illuminate when the gas sample line is present and/or functioning properly. In some aspects, the patient gas sample line graphic 818 may dim when the gas sample line is not present or not properly functioning. In some aspects, the sample tee graphic 820 may illuminate when the sample tee is present and/or functioning properly. The sample tee graphic 820 may display an error message, a not functioning properly message, or a not present message when the sample tee is functioning in error, not functioning properly, and/or not present. For example, the sample tee graphic 820 may dim when the sample tee is not present, not functioning properly, and/or has an error.

Continuing with FIG. 8B, the patient wye graphic 824 may display a status of the patient wye. For example, the patient wye graphic 824 may illuminate when the patient wye is functioning properly and/or present. In some aspects, the patient wye graphic 824 may dim when the patient wye is not present, not functioning properly, and/or has an error. The patient icon 826 may display a status of the patient. For example, the patient icon 826 may display that the patient is receiving therapeutic gas, not receiving therapeutic gas, receiving an incorrect amount of therapeutic gas, or the patient's vitals. The breathing circuit expiratory limb graphic 828 may display a status of the breathing circuit expiratory limb. In some aspects, the breathing circuit expiratory limb graphic 828 may illuminate when the breathing circuit expiratory limb is present and/or functioning properly. In some aspects, the breathing circuit expiratory limb graphic 828 may dim when the breathing expiratory limb is not present, not functioning properly, and/or has an error. The breathing circuit inspiratory limb graphic 830 may display a status of the breathing circuit inspiratory limb. In some aspects, the breathing circuit inspiratory limb graphic 830 may illuminate when the breathing circuit inspiratory limb is present and/or functioning properly. In some aspects, the breathing circuit inspiratory limb graphic 830 may dim when the breathing circuit inspiratory limb is not present, not functioning properly, and/or has an error.

Continuing with FIG. 8B, the injector module graphic 832 may display a status of the injector module. In some aspects, the injector module graphic 832 may illuminate when the injector module is present and/or properly functioning. In some aspects, the injector module graphic 832 may dim when the injector module is not present, not properly functioning, and/or has an error. The injector module graphic 832 may display a flow rate (or flow rates) through the injector module. The injector module graphic 832 may display a message or indication that the injector module is properly connected to a power source. The delivery conduit graphic 834 may display a status of the delivery conduit. In some aspects, the delivery conduit graphic 834 may illuminate when the delivery conduit is present and/or functioning properly. In some aspects, the delivery conduit graphic 834 may dim when the delivery conduit is not present, not functioning properly, and/or has an error.

FIG. 8C illustrates one example of a dose tile 806. The dose tile 806 illustrates a secondary delivery subsystem delivery circuit flow diagram. The dose tile 806 may include, for example, a tile name 816, a secondary delivery subsystem on/off indicator 836, an expand tile button 822, an O₂ flow rate graphic 838, an O₂ flow indicator graphic 840, an O₂ flowmeter graphic 842, an O₂ tubing inlet graphic 844 (e.g., to secondary gas delivery subsystem inlet), an therapeutic gas delivery system graphic 812, an O₂ tubing outlet graphic 846 (e.g., from secondary gas delivery subsystem outlet), a manual resuscitator graphic 848, an secondary delivery subsystem default dose graphic 850, a primary and secondary delivery subsystem dose graphic 852, and/or a primary delivery status indicator 854.

Continuing with FIG. 8C, the O₂ flow rate graphic 838 may display the O₂ flow rate (e.g., a numerical value). The O₂ flow indicator graphic 840 may display a status of the O₂ flow. In some aspects, the O₂ flow indicator graphic 840 may illuminate when O₂ is present, flowing, and/or properly functioning. In some aspects, the O₂ flow indicator graphic 840 may dim when the O₂ flow is not present, not properly functioning, and/or has an error. The O₂ tubing outlet graphic 846 may display a status of the O₂ tubing outlet. In some aspects, the O₂ tubing outlet graphic 846 may illuminate when the O₂ tubing outlet is present and/or functioning properly. In some aspects, the O₂ tubing outlet graphic 846 may dim when the O₂ tubing outlet is not present, not properly functioning, and/or has an error. The manual resuscitator graphic 848 may display a status of the manual resuscitator. In some aspects, the manual resuscitator graphic 848 may illuminate when the manual resuscitator is present and/or functioning properly. In some aspects, the manual resuscitator graphic 848 may dim when the manual resuscitator is not present, not properly functioning, and/or has an error. The secondary delivery subsystem default dose graphic 850 may display the secondary delivery subsystem default dose (e.g., in parts-per-million). The primary and secondary delivery subsystem dose graphic 852 may display the primary and secondary delivery subsystem doses (e.g., when non-zero). The primary delivery status indicator 854 may display a status of the primary delivery. For example, the primary delivery status may be present, not present, properly functioning, not properly functioning, has an error.

FIGS. 8A-8C are exemplary embodiments of display graphics 800. The components of the individually described display graphics 800 may be applied to the other display graphics 800 without limitation. Additionally, components, indications, and/or statuses may be displayed in different locations and/or in different combinations without departing from this disclosure.

FIGS. 9A-9D illustrate examples of device icons 900 (e.g., graphics). The device icons 900 may include, for example, one or more controls 902 (as illustrated in FIG. 9A), one or more indicators 904 (as illustrated in FIG. 9B), one or more symbols 906 (as illustrated in FIG. 9C), and/or one or more compressed gas cylinder images 908. The central display 102, status display 112(a), and/or status display 112(b) of the therapeutic gas delivery system 100 may display one or more device icons 900. For example, the central display 102 may display a visual alarm. In some examples, one or more display graphics 800 (as illustrated for example in FIGS. 8A-8C) may include one or more device icons 900 (as illustrated for example in FIGS. 9A-9D).

FIG. 9A illustrates examples of controls 902 (e.g., buttons). Selecting (e.g., pressing, pushing) a specific control 902 may cause a corresponding specific action of the therapeutic gas delivery system 100. The controls 902 may include expand tile, collapse tile, confirm, cancel/dismiss, cancel/dismiss and terminate process, unselected option, selected option, backspace/delete, help, information, previous, next, page up, page down, return, restart, snooze alarm, silence alarm, decrease volume, increase volume, decrease brightness, increase brightness, and/or hide keyboard. In some aspects, the central display 102 (as illustrated in FIG. 7) may display one or more controls 902 (as illustrated in FIG. 9A). In some aspects, one or more controls 902 (as illustrated in FIG. 9A) may be included in one or more of the display graphics 800 (as illustrated in FIGS. 8A-8C). For example, the expand tile (as illustrated in FIG. 9A) may be the expand tile button 822 (as illustrated in FIGS. 8B-8C).

FIG. 9B illustrates examples of indicators 904 (e.g., status indicators). Each specific indicator 904 may display a corresponding status of one or more components of the therapeutic gas delivery system 100. The indicators 904 may include battery level, connected to AC power source and battery charging, connected to AC power source and battery fully charged, battery failure, task pending, task in progress, failure, warning, success, primary delivery is idle, primary delivery is on, primary delivery is stopped/interrupted, initial dose, dose change, secondary delivery subsystem delivery started, secondary delivery subsystem delivery stopped, indicator arrow, low priority alarm, and/or high priority alarm. In some aspects, the central display 102 (as illustrated in FIG. 7) may display one or more indicators 904 (as illustrated in FIG. 9B). For example, the battery level (as illustrated in FIG. 9B) may be displayed in status bar 708 (as illustrated in FIG. 7). In some aspects, one or more indicators 904 (as illustrated in FIG. 9B) may be included in one or more of the display graphics 800 (as illustrated in FIGS. 8A-8C). For example, primary delivery is on (as illustrated in FIG. 9B) may be the primary delivery status indicator 854 (as illustrated in FIG. 8C).

FIG. 9C illustrates examples of symbols 906. Each specific symbol 906 may provide corresponding specific information about the therapeutic gas delivery system 100. The symbols 906 may include shut down, reboot, pre-use checkout, pre-use checkout failed, pre-use checkout warning, pre-use checkout passed, alarm history, alarm history new alarms from review (e.g., 3 new alarms), new alarm for review, treatment history, treatment ID, help, patient ID, patient profile patient ID missing, patient profile patient ID supplied, patient profile all patient data successfully input, system maintenance replace sample subsystem, system maintenance sample subsystem successfully replaced, system maintenance, and/or system settings. In some aspects, the central display 102 (as illustrated in FIG. 7) may display one or more symbols 906 (as illustrated in FIG. 9C). For example, the alarm history (as illustrated in FIG. 9C) may be displayed on the central display 102 (as illustrated in FIG. 7). In some aspects, one or more symbols 906 (as illustrated in FIG. 9C) may be included in one or more of the display graphics 800 (as illustrated in FIGS. 8A-8C).

FIG. 9D illustrates examples of compressed gas cylinder images 908. Each specific compressed gas cylinder image 908 may display a status of a corresponding gas cylinder. The compressed gas cylinder images 908 may include cylinder is idle, cylinder is delivering, cylinder low priority alarm, cylinder high priority alarm, cylinder bay failure, and/or cylinder not detected. In some aspects, the central display 102 (as illustrated in FIG. 7) may display one or more compressed gas cylinder images 908 (as illustrated in FIG. 9D). In some aspects, one or more compressed gas cylinder images (as illustrated in FIG. 9D) may be included in one or more of the display graphics 800 (as illustrated in FIGS. 8A-8B).

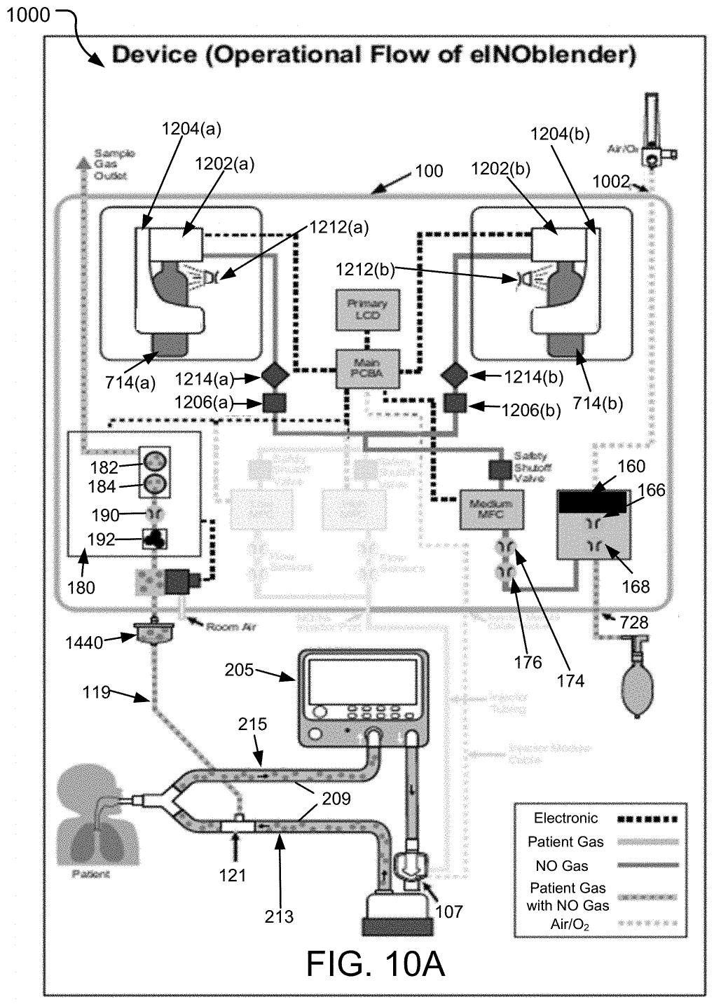

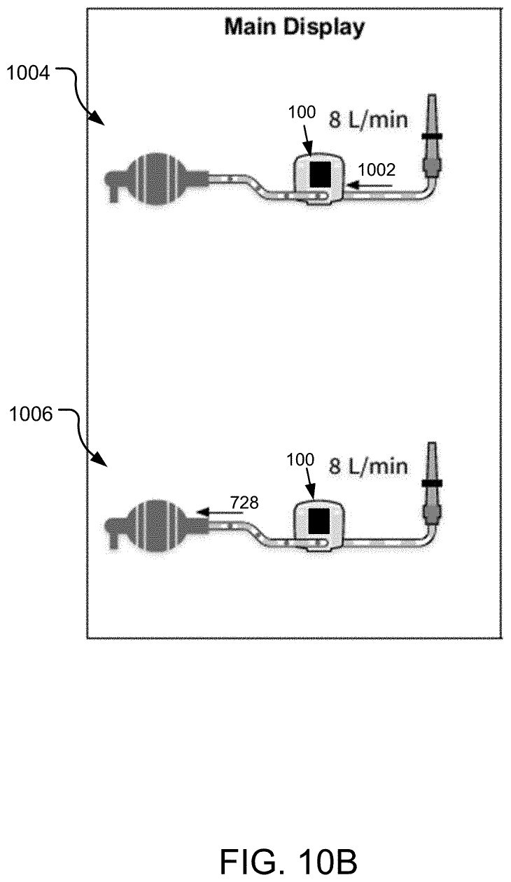

FIG. 10A illustrates an exemplary embodiment of the secondary delivery subsystem operational flow 1000 and FIG. 10B illustrates exemplary graphics of the secondary delivery subsystem flow control 1004, 1006. One example of the secondary delivery subsystem operational flow 1000 is illustrated in FIG. 10A. In some aspects, the secondary delivery subsystem operational flow 1000 (as illustrated for example in FIG. 10A) may be used in conjunction with the secondary delivery subsystem 160 (as illustrated for example in FIGS. 1, 2, and 5). In some aspects, one or more features, components, or elements illustrated in the secondary delivery subsystem operational flow 1000 (as illustrated in FIG. 10A) may be the same as or similar to one or more features, components, or elements of the secondary delivery subsystem 160 (as illustrated for example in FIGS. 1, 2, and 5).

In at least one example, the secondary delivery subsystem may include an inlet connector that is a tapered, barbed fitting. Similarly, the secondary delivery subsystem may include an outlet connector that is a tapered, barbed fitting. The secondary delivery subsystem may include a minimum flow rate of approximately 2 L/min and/or may include a maximum flow rate of 20 L/min. The secondary delivery subsystem may include a maximum pressure of approximately 6.1 psig, a vent relief pressure of approximately 5.5+/−10% psig, and/or a relief pressure of 45-55 psig. The secondary delivery subsystem may include NO₂ (by-product) of less than or equal to 1 ppm at a dose of 10 ppm NO into 98-100% O₂ via O₂ flow rate of approximately 10 L/min. The secondary delivery subsystem may include a dose range of approximately 0.1-80 ppm.

FIG. 10A illustrates one example of the secondary delivery subsystem operational flow 1000. The secondary delivery subsystem 160 within the therapeutic gas delivery system 100 may serve as a backup delivery device and may provide the user with a method to deliver therapeutic gas through a manual resuscitator to the patient when clinically appropriate or in the event of the primary delivery system failure. For example, the secondary delivery subsystem is an integrated electronic blender that may function as a backup delivery device to provide an adjustable therapeutic gas dose with user-supplied air/O₂ to a manual resuscitator or gas delivery system. The electronic blender incorporates a separate control and delivery pathway that serves as a redundant mechanism for nitric oxide delivery in the event of main system fault.

The secondary delivery subsystem operational flow 1000 may include a therapeutic gas delivery system 100, a secondary delivery subsystem inlet 1002, secondary delivery subsystem flow sensors 174 and 176, and/or a secondary delivery subsystem outlet 726.

The following secondary delivery subsystem operational flow 1000 is illustrated in FIG. 10A. Flow (e.g., cylinder, wall air, O₂) may enter the secondary delivery subsystem inlet 1002. The flow rate (e.g., air, O₂) may be measured internally by one or more flow sensors 166 and 168. A proportional amount of therapeutic gas may be added to the flow (e.g., air, O₂) inside the secondary delivery subsystem 160. The mixture may exit the therapeutic gas delivery system 100 through the secondary delivery subsystem outlet 726 and may pass through O₂ tubing to a manual resuscitator.

In some examples, the secondary delivery subsystem may automatically activate when the internal flow sensors 166 and 168 detect flow (e.g., air, O₂) from a gas supply that exceeds 2 L/min for more than 2 seconds. In some examples, the secondary delivery subsystem may automatically deactivate when the flow (e.g., air, O₂) drops below 1 L/min for more than 2 seconds.

Whenever the primary delivery dose is set greater than zero, the secondary delivery subsystem dose may automatically match the set dose. If the primary delivery dose is reduced to zero, the secondary delivery subsystem default dose may be reset to 20 ppm. During secondary delivery subsystem use, the secondary delivery subsystem default dose may be set to 20 ppm when the primary delivery dose is set to zero. The validated flow range for use with the secondary delivery subsystem may be 2-20 L/min.

In some aspects, the secondary gas delivery subsystem 160 may deliver therapeutic gas to the patient. The delivery may start at a compressed gas cylinder 714(a) or 714(b). The compressed gas cylinder may be attached to a cylinder manifold 1202(a) and 1202(b) through a cylinder loading mechanism. Using QR code scanning technology 1212(a) and 1212(b), the therapeutic gas delivery system may read and verify product code, expiration date, lot number, and/or cylinder concentration. Therapeutic gas may enter the secondary gas delivery subsystem 160 by passing from a compressed gas cylinder 714 (e.g., 714(a), 714(b) and through a respective filter 1214 (e.g., 1214(a), 1214(b)) and a respective safety shutoff valve 1206 (e.g., 1206(a), 1206(b)). Therapeutic gas may be delivered to the patient through the injector module 107. A ventilator 205 may be used to circuit breathing gas through the breathing circuit 209. The breathing gas, air and therapeutic gas, may be delivered to the patient through the inspiratory limb 213 and circulated back from the patient to the ventilator 205 through the expiratory limb 215.

The breathing gas may be tested in a gas analyzing subsystem 180. The sample gas may be pumped by a sample pump 192 from the inspiratory limb 213 at a gas sample tee 121. The sample gas may travel through the patient gas sample line 119 through a patient gas sample filter 1440. The patient gas sample filter 1440 may filter the sample gas before entering the gas analyzing subsystem 180. The gas analyzing subsystem 180 may include gas concentration sensors 182 and 186 and a gas concentration flow sensor 190. The resulting values may be displayed on the central display 102, such as on a monitoring tile displayed on the central display 102.

In some examples, the secondary delivery subsystem is for short-term, attended use when the primary delivery device may not practicably be used. In some examples, the secondary delivery subsystem is not intended for use as a primary therapeutic gas delivery system for long-term use.

FIG. 10B illustrates exemplary graphics 1004, 1006 of the secondary delivery subsystem flow control. The top graphic 1004 illustrates an example of the secondary delivery subsystem flow control inlet and the bottom graphic 1006 illustrates an examples of the secondary delivery subsystem flow control outlet. For example, the top graphic 1004 illustrates air/O₂ flowing into the therapeutic gas delivery system 100 through the secondary delivery subsystem inlet 1002. The bottom graphic 1006 shows air/O₂ flowing out of the therapeutic gas delivery system 100 at the secondary delivery subsystem outlet 726.

In some aspects, one or more of the exemplary graphics 1004, 1006 of the secondary delivery subsystem flow control (as illustrated for example in FIG. 10B) may correlate with the secondary delivery subsystem operational flow 1000 (as illustrated for example in FIG. 10A). For example, FIGS. 10A-10B both illustrate both the secondary delivery subsystem inlet 1002 and the secondary delivery subsystem outlet 726. In some aspects, one or more of the exemplary graphics 1004 of the secondary delivery subsystem flow control (as illustrated for example in FIG. 10B), 1006 may be displayed in the dose tile 806 (as illustrated for example in FIG. 8C).

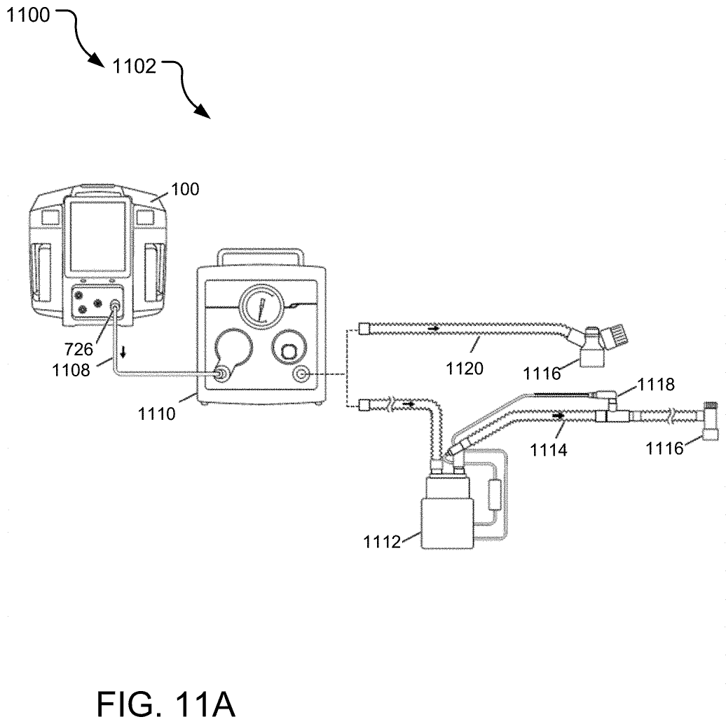

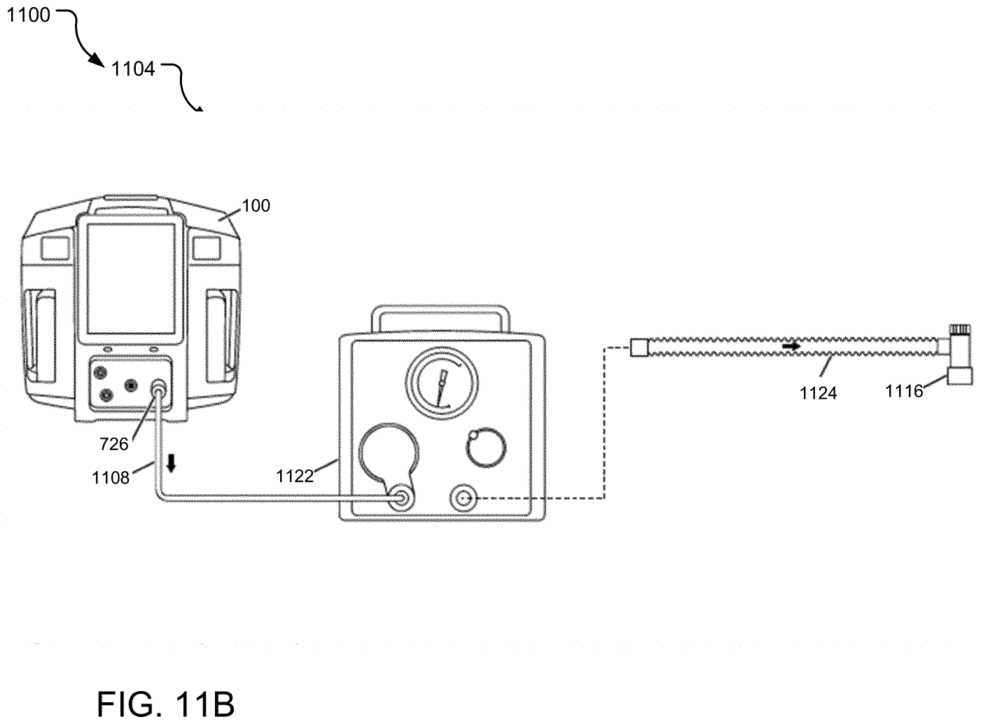

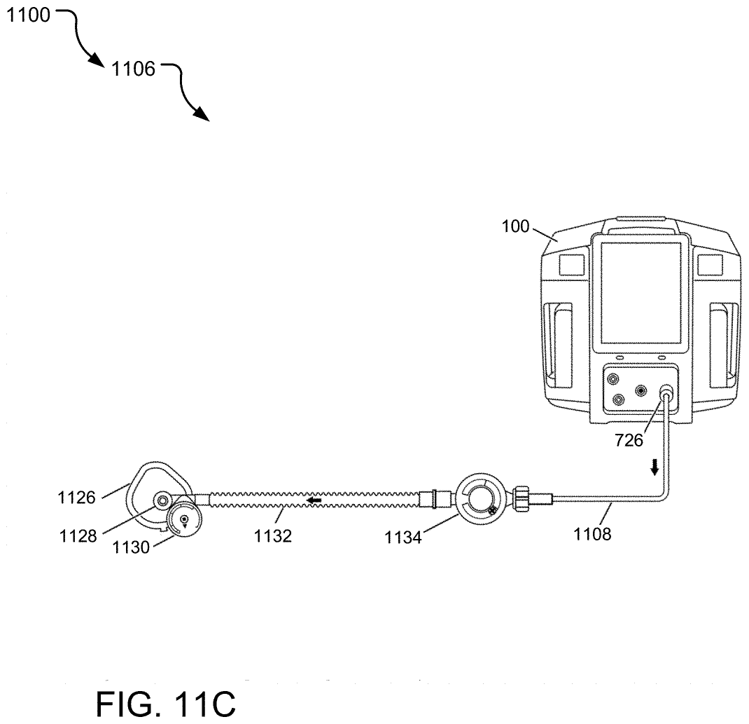

FIGS. 11A-11C illustrate diagrams of examples of breathing circuits 1100, which may be used with the secondary delivery subsystem. In some aspects, the secondary delivery subsystem that may be used with breathing circuit 1100 (as illustrated for example in FIGS. 11A-11C) may have one or more same or similar features, components, or elements as the secondary delivery subsystem 160 (as illustrated for example in FIGS. 1, 2, and 5). In some aspects, a breathing circuit 1100 (as illustrated for example in FIGS. 11A-11C) may be used, in whole or in part, to replace breathing circuit 209 (as illustrated for example in FIG. 10A).

FIGS. 11A-11C are exemplary embodiments of breathing circuits 1100. The components of the individually described breathing circuits 1100 may be applied to the other breathing circuits 1100 without limitation. Additionally, the breathing circuits 1100 (as illustrated for example in FIGS. 11A-11C) may include one or more same or similar features, components, or elements as the breathing circuit 209 (as illustrated for example in FIG. 1 and FIG. 3). In some aspects, FIGS. 11A-11C illustrate diagrams of breathing circuits 1100. In some aspects, the central display 102 (as illustrated for example in FIG. 7) may display one or more diagrams of breathing circuit 1100 (as illustrated in FIGS. 11A-11C).

As previously indicated, the secondary delivery subsystem (e.g., secondary delivery subsystem 160 as illustrated for example in FIGS. 1, 2, and 5) may be used with breathing circuit 1100. For example, the secondary delivery subsystem may be used with a manual resuscitation bag. The additional resuscitation devices for the secondary delivery subsystem may include a T-Piece Resuscitator Circuit 1102 (as illustrated in FIG. 11A), an Infant Resuscitator Circuit 1104 (as illustrated in FIG. 11B), and an Infant T-Piece Resuscitator Circuit 1106 (as illustrated in FIG. 11C).

To use the secondary delivery subsystem with a manual resuscitation bag, O₂ tubing may be connected from an oxygen flowmeter or air/O₂ blender to the secondary delivery subsystem inlet. The secondary delivery subsystem inlet may be located on the back of the therapeutic gas delivery system 100. Additionally, O₂ tubing may be connected from the secondary delivery subsystem outlet 726 to a manual resuscitator. The flowmeter may be adjusted to the desired gas flow rate (e.g., 2-20 L/min) for use with the resuscitation bag. The secondary delivery subsystem may automatically activate, delivering therapeutic gas at the set (or default) dose. Delivery may continue through the primary delivery device (if available). If the wall gas flow rate is too high a notification may display indicating an excessive flow through the secondary delivery subsystem.

The secondary delivery subsystem flow rate on the central display 102 may be verified to be consistent with the reading on the flowmeter. The secondary delivery subsystem outlet port may be verified that it illuminates, indicating therapeutic gas delivery. The manual resuscitator may be squeezed several times to purge NO₂ from the system. The manual resuscitation bag may be squeezed to purge NO₂ prior to connecting it to the patient. Additionally, the manual resuscitation bag may be squeezed repeatedly during use to prevent NO₂ building up in the manual resuscitation bag. Instructions may be displayed on the therapeutic gas delivery system 100 on central display 102. The manual resuscitation bag may be removed from the patient when not in use to prevent NO₂ accumulating. The flowmeter may be turned off when finished using the manual resuscitator.

FIG. 11A illustrates an example of a T-piece resuscitator circuit 1102, which is an example of a breathing circuit 1100. The T-piece resuscitator circuit 1102 may include, for example, a therapeutic gas delivery system 100, an secondary delivery subsystem outlet 726, O₂ tubing 1108, T-piece resuscitator 1110, a humidifier 1112, a humidified resuscitation system circuit 1114, one or more patient connections 1116, a temperature probe 1118, and/or a T-piece circuit 1120 (e.g., with duckbill port).

Continuing with FIG. 11A, the secondary delivery subsystem may deliver breathing gas to a breathing circuit through a secondary delivery subsystem outlet 726 (e.g., from the secondary delivery subsystem 160). The breathing gas may then flow through O₂ tubing 1108 into a T-piece resuscitator 1110. After passing through the T-piece resuscitator 1110, the breathing gas may flow through a T-piece circuit 1120 and delivered to a patient at a patient connection 1116. In another embodiment, breathing gas may enter a humidifier 1112 after passing through the T-piece resuscitator 1110. Once the breathing gas passes through the humidifier 1112, the breathing gas may pass through a humidified resuscitation circuit 1114 and delivered to a patient at a patient connection 1116. A temperature probe 1118 may be positioned within the humidified resuscitation system circuit 1114 to measure the temperature of the breathing gas being delivered to a patient.

FIG. 11B illustrates an example of an Infant Resuscitator Circuit 1104, which is an example of a breathing circuit 1100. The Infant Resuscitator Circuit 1104 may include a therapeutic gas delivery system 100, a secondary delivery subsystem outlet 726, O₂ tubing 1108, infant resuscitator 1122, corrugated tubing with pressure control 1124, and/or a patient connection 1116.

Continuing with FIG. 11B, the secondary delivery subsystem may deliver breathing gas to a breathing circuit through a secondary delivery subsystem outlet 726 (e.g., from the secondary delivery subsystem 160). The breathing gas may flow through O₂ tubing 1108 into an infant resuscitator 1122. Once through the infant resuscitator 1122, the breathing gas may enter corrugated tubing with pressure control 1124 and be delivered to the patient at a patient connection 1116.

FIG. 11C illustrates an example of an Infant T-piece Resuscitator Circuit 1106, which is an example of a breathing circuit 1100. The Infant T-piece Resuscitator Circuit 1106 may include, for example, an therapeutic gas delivery system 100, a face mask 1126 (e.g., neonate or infant facemask), a thumb port 1128 (e.g., patient connection, positive end expiratory pressure (“PEEP”) adjustment, and pressure delivery), a pressure manometer 1130, corrugated tubing 1132, an in-line PIP controller 1134, O₂ tubing 1108, and/or a secondary delivery subsystem outlet 726.

Continuing with FIG. 11C, the secondary delivery subsystem may deliver breathing gas to a breathing circuit through a secondary delivery subsystem outlet 726 (e.g., from the secondary delivery subsystem 160). The breathing gas may flow through O₂ tubing 1108 into an in-line PIP controller 1134. The breathing gas may then flow through corrugated tubing 1132 and pass through a pressure manometer 1130. The breathing gas may be delivered to the patient through a thumb port 1128 and/or a face mask 1126.

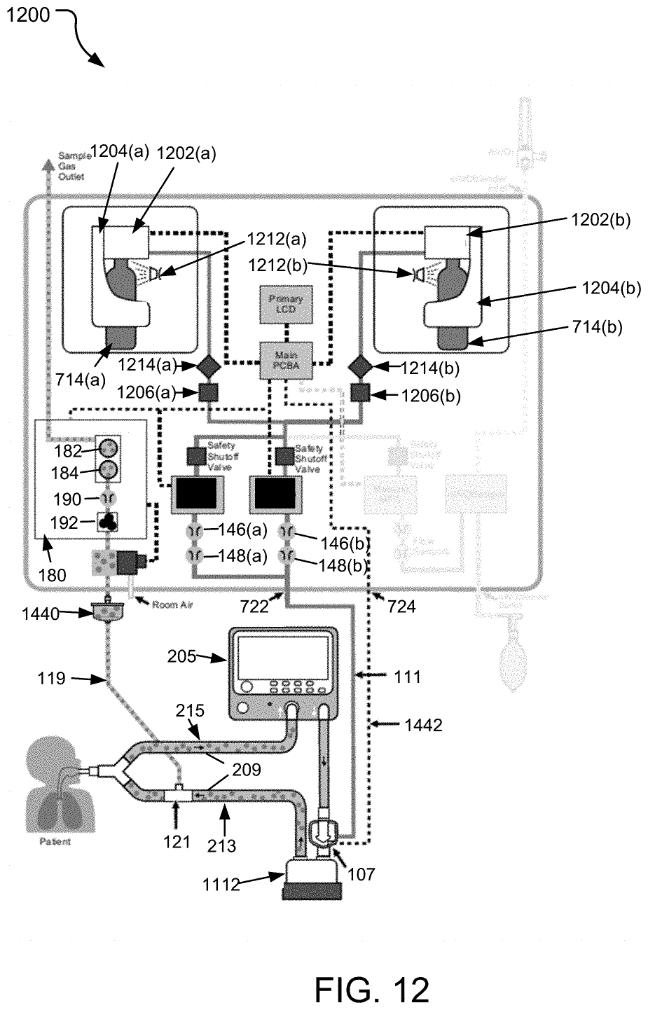

FIG. 12 illustrates an exemplary embodiment of the therapeutic gas delivery system operational flow 1200. In some aspects, the therapeutic gas delivery system operational flow 1200 (as illustrated in FIG. 12) may be used in conjunction with the therapeutic gas delivery system 100 (as illustrated for example in FIGS. 1, 2, and 5). In some aspects, one or more features, components, or elements illustrated in the therapeutic gas delivery system operational flow 1200 (as illustrated in FIG. 12) may be the same as or similar to one or more features, components, or elements of the therapeutic gas delivery system 100 (as illustrated for example in FIGS. 1, 2, and 5).

In at least one example, the delivery of NO may be uninterrupted. In the case of interruption, the system may provide a visual and/or audible indication to the user that delivery is interrupted. The therapeutic gas delivery system may include a dual channel design to ensure the safe delivery of therapeutic gas to the patient. The dual channel approach may permit therapeutic gas delivery independent of monitoring.

The following exemplary embodiment of the therapeutic gas delivery system operational flow 1200 is illustrated in FIG. 12. The therapeutic gas delivery may start at one or more of the compressed gas cylinders 714 (e.g., 714(a), 714(b)). The compressed gas cylinder 714 may be attached to a respective cylinder manifold 1202 (e.g., 1202(a), 1202(b)) through a cylinder loading mechanism 1204 (e.g., 1204(a), 1204(b)). Using QR code scanning technology 1212 (e.g., 1212(a), 1212(b)), the therapeutic gas delivery system may read and verify product code, expiration date, lot number, and/or cylinder concentration. Therapeutic gas may enter the therapeutic gas delivery system from a compressed gas cylinder 714 and pass through a respective filter 1214 (e.g., 1214(a), 1214(b)) and a respective safety shutoff valve 1206 (e.g., 1206(a), 1206(b)). The safety valve 1206 may remain open during normal operation. An injector module 107 may be placed in the breathing circuit on the inspiratory inlet of the humidifier. Therapeutic gas may enter the injector module 107 by passing through the injector module outlet 722, into the delivery conduit 111, then entering the injector module 107. The injector module 107 may be supplied electricity through the injector module cable socket 724 and the injector module electrical cable 1442. The injector module flow sensors (not shown in FIG. 12) may measure the breathing circuit flow rate in real time. The therapeutic gas delivery system may receive this information and, based on the measured flow, the cylinder concentration, and the set dose, may calculate a proportional flow of high concentration therapeutic gas to be delivered in real time into the breathing circuit via the injector module 107. A humidifier 1112 may be used to humidify the breathing gas before entering the breathing circuit 209.

The high concentration of therapeutic gas may pass through the therapeutic gas delivery conduit 111 into the injector module 107, and into the inspiratory limb 213 of the breathing circuit 209. The breathing circuit flow may dilute the high concentration therapeutic gas to achieve the set dose. There may be two therapeutic gas mass flow controllers in the primary delivery system; a low flow controller 1208 and a high flow controller 1210. These flow controllers 1208, 1210 may facilitate the proportional delivery of therapeutic gas. Each flow controller 1208, 1210 may be monitored, respectively, by two internal therapeutic gas flow sensors 146, 148 (e.g., 146(a), 146(b), 148(a), 148(b)) in the therapeutic gas delivery system to monitor delivered therapeutic gas flow to the injector module 107. The therapeutic gas delivery system may continually adjust the volume of therapeutic gas injected to the circuit, to deliver a constant dose of therapeutic gas to the patient independent of breath rates and varying inspiratory flow rates. The gas monitoring system may measure the delivered inspired NO and NO₂ concentration in the breathing circuit. A zero valve may allow for low calibrations to occur while the sample line 119 is connected to the breathing circuit 209.

In at least one instance, when the gas monitoring system measures the delivered inspired NO and NO₂ concentration in the breathing circuit, a sample pump 192 may draw approximately a volumetric flow rate of gas from the breathing circuit 209 using the patient gas sample line 119 and filter 1440. For example, the volumetric flow rate drawn by the sample pump 192 may be about 0.23 L/min of gas. In another example, the volumetric flow rate drawn by the sample pump 192 may be about 0.1 L/min of gas to about 0.3 L/min of gas. The gas is drawn into the patient gas sample line 119 through a gas sample tee 121 located in the inspiratory limb 213 of the breathing circuit 209. The gas may pass through the gas analyzing subsystem 180, which may house the gas concentration sensors 182 and 186 where it is analyzed. A gas concentration flow sensor 190 may analyze the flow rate of the gas in the gas analyzing subsystem 180. The resulting values may be displayed on the central display 102, such as on a monitoring tile displayed on the central display 102.

In at least one example, the therapeutic gas delivery system may monitor nitric oxide in the range of approximately 0-10 ppm, with a resolution of approximately 0.1, and an accuracy of +/−20% of reading+0.5 ppm. In some examples, the therapeutic gas delivery system may monitor nitric oxide in the range of approximately 10-100 ppm, with a resolution of approximately 1, and an accuracy of +/−10% of reading+0.5 ppm. The therapeutic gas delivery system may monitor nitrogen dioxide in the range of approximately 0-10 ppm, with a resolution of approximately 0.1, and an accuracy of +/−20% of reading or 0.5 ppm, whichever is greater. The therapeutic gas delivery system may monitor oxygen in the range of approximately 18-100% v/v, with a resolution of approximately 1, and an accuracy of +/−3% v/v.

In some examples, the maximum breathing circuit pressure may be approximately 150 cm H₂O. In some examples, the low calibration may be daily zero. In some examples, the rise time may be approximately 30 (10-90%) seconds. In some examples, the sample flow rate may be approximately 230+/−10% mL/min.

In some examples, a ventilator 205 may be connected to the expiratory limb 215 of the breathing circuit 209 on one end and connected to the injector module 107 on the other end. In some aspects, the ventilator 205 may pump breathing gas to the patient.

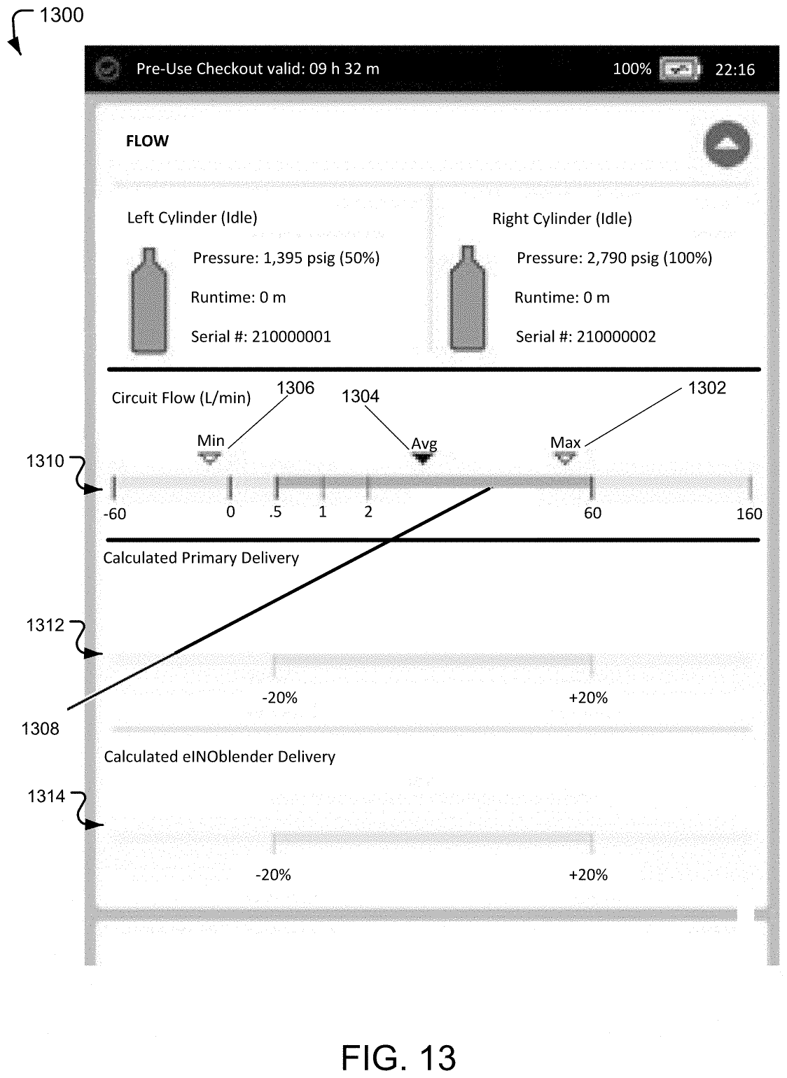

FIG. 13 illustrates one exemplary embodiment of flow tile information 1300. In some aspects, the central display 102 (as illustrated in FIG. 7) may display the flow tile information 1300 (as illustrated in FIG. 13). In some examples, pressing (e.g., touching) the expand tile button 822 (as illustrated in FIGS. 8B-8C) may cause the central display 102 to display the flow tile information 1300 (as illustrated in FIG. 13). In other words, the flow tile information 1300 (as illustrated in FIG. 13) may be viewed by pressing the expand tile button 822 (as illustrated in FIGS. 8B-8C).

As illustrated in FIG. 13, the flow tile information 1300 may display, for example, a circuit flow graph 1310, a calculated primary delivery graph 1312, and/or a calculated secondary delivery subsystem delivery graph 1314.

In some aspects, the circuit flow graph 1310 may display a maximum flow rate indicator 1302, an average flow rate indicator 1304, and/or a minimum flow rate indicator 1306. For example, the maximum flow rate indicator 1302 may indicate the maximum flow rate measured by the injector module when placed within the breathing circuit, the average flow rate indicator 1304 may indicate the average flow rate measured by the injector module when placed within the breathing circuit, and the minimum flow rate indicator 1306 may indicate the minimum flow rate measured by the injector module when placed within the breathing circuit.

In some aspects, the circuit flow graph 1310, calculated primary delivery graph 1312, and/or calculated secondary delivery subsystem delivery graph 1314 may each be illuminated or not illuminated. In some examples, the calculated primary delivery graph 1312 and the calculated secondary delivery subsystem delivery graph 1314 are not illuminated (e.g., dimmed) if a dose has not been set. In some examples, if the cylinders are loaded, cylinder pressure, run time, and/or serial number may be displayed next to both the left and right cylinder icons.

In some examples, illuminated indicators pertaining to the global status indicator, satellite screens, port indicator, cylinders and molecules in the flow tile, and primary delivery on indicator, indicate that a dose has been set and confirmed. In an example, the illuminated indicators may have a color. For example, the illuminated indicators may be teal.

In some examples, a minimum flow rate indicator 1306 to the left of zero this indicates that reverse flow is detected. In some examples, a maximum flow rate indicator 1302 in the right-hand (e.g., yellow) region indicates that the delivery dose may be limited during peak inspiratory flow. In some examples, a region 1308 (e.g., a green colored region) on the circuit flow graph 1310 indicates the range of targeted positive flow rate through the injector module. In other words, the range of targeted positive flow rate may be demonstrated by a region 1308 displayed on the circuit flow graph 1310.

FIGS. 14A-14R illustrate diagrams of examples of breathing circuits 1400, which may be used with the therapeutic gas delivery system. In some aspects, the therapeutic gas delivery system that may be used with breathing circuit 1400 (as illustrated for example in FIGS. 14A-14R) may have one or more same or similar features, components, or elements as the therapeutic gas delivery system 100 (as illustrated for example in FIGS. 1, 2, and 5). In some aspects, a breathing circuit 1400 (as illustrated for example in FIGS. 14A-14R) may be used, in whole or in part, to replace breathing circuit 209 (as illustrated for example in FIG. 12).

FIGS. 14A-14R are exemplary embodiments of breathing circuits 1400. The components of the individually described breathing circuits 1400 may be applied to other breathing circuits 1400 without limitation. Additionally, the breathing circuits 1400 (as illustrated for example in FIGS. 14A-14R) may include one or more same or similar features, components, or elements as the breathing circuit 209 (as illustrated for example in FIG. 1 and FIG. 3). In some aspects, FIGS. 14A-14R illustrate diagrams of breathing circuits 1400. In some aspects, the central display 102 (as illustrated for example in FIG. 7) may display one or more diagrams of breathing circuit 1400 (as illustrated in FIGS. 14A-14R).

As previously discussed, the therapeutic gas delivery system 100 may include an injector module outlet 722, an injector module cable socket 724, a secondary delivery subsystem outlet 726, and/or a sample line inlet 728, as illustrated for example in FIG. 7.

The injector module outlet 722 may be configured to deliver therapeutic gas from the therapeutic gas delivery system 100. For example, a therapeutic gas delivery conduit 111 may establish fluid communication (e.g., a fluid flow path) between the therapeutic gas delivery system 100, at the injector module outlet 722, and the injector module 107.

The injector module cable socket 724 may be configured to attach the injector module electrical cable 1442 to the injector module 107, to provide electricity to the injector module 107. For example, the injector module cable socket is connected to the therapeutic gas delivery system 100. When the injector module electrical cable 1442 is connected to the injector module cable socket 724 on the therapeutic gas delivery system on one end and the injector module 107 on the other end, the injector module 107 is provided with electricity.

The secondary delivery subsystem outlet 726 may be used to deliver therapeutic gas by the secondary gas delivery subsystem 160 in the event of failure of the therapeutic gas delivery system 100.

The sample line inlet 728 may be configured to receive a sample of the gas being delivered to the patient. For example, a sample of the gas may be taken out of the inspiratory limb 213 of the breathing circuit at a gas sample tee 121. The sample gas may then flow through a patient gas sample line 119 and a patient gas sample filter 1440. The sample gas may enter a gas analyzing subsystem 180 at sample line inlet 728. The patient gas sample filter 1440 may filter humidity out of the gas for more accurate testing. The sample gas may be tested by gas concentration sensors and gas flow sensors. A pump may be used to draw the sample gas from the inspiratory limb 213 at the gas sample tee 121 through the patient gas sample line 119 and filter 1440 to the gas analyzing subsystem 180.

In some examples, as illustrated in FIGS. 14A-14J, the therapeutic gas delivery system 100 may be used with (e.g., connected to) typical gas delivery systems. For example, the therapeutic gas delivery system may be used in the following types of breathing circuits 1400: a conventional ventilator circuit 1402 (as illustrated in FIG. 14A), an anesthesia circuit 1404 (as illustrated in FIG. 14B), a spontaneous circuit 1406 (as illustrated in FIG. 14C), a nasal cannula circuit 1408 (as illustrated in FIG. 14D), a High Frequency Jet Ventilation Circuit 1410 (as illustrated in FIGS. 14E-14G), a High Frequency Oscillatory Ventilator Unfiltered Circuit 1412 (as illustrated in FIG. 14H), a dual-limb transport ventilator circuit 1414 (as illustrated in FIGS. 14I-14J), and/or a single-limb transport ventilator circuit 1416 (as illustrated in FIG. 14K).

In some examples, as illustrated in FIGS. 14L-14R, the therapeutic gas delivery system 100 may be used with (e.g., connected to) additional gas delivery systems. For example, the therapeutic gas delivery system 100 may be used in the following types of additional breathing circuits 1400: a High Frequency Oscillatory Ventilator Filtered Circuit 1418 (as illustrated in FIG. 14L), an Infant Flow Circuit 1420 (as illustrated in FIG. 14M), a Precision Flow Circuit 1422 (as illustrated in FIG. 14N), a Continuous Positive Airway Pressure System Circuit 1424 (as illustrated in FIG. 14O), a High Flow Breathing Circuit 1426 (as illustrated in FIG. 14P), a High Flow Infant Circuit Nasal Cannula Circuit 1428 (as illustrated in FIG. 14Q), and a High Flow Nasal Cannula Oxygen Therapy Circuit 1430 (as illustrated in FIG. 14R).

FIG. 14A illustrates one example of a conventional ventilator circuit 1402, which may include a therapeutic gas delivery system 100 and a ventilator 205 (e.g., conventional ventilator). The therapeutic gas delivery system 100 may include, as best illustrated in FIG. 7, an injector module outlet 722, an injector module cable socket 724, and/or a sample line inlet 728. The ventilator 205 may include, as illustrated in FIG. 14A, a ventilator expiratory port 1444 and a ventilator inspiratory port 1446.

Additionally, the conventional ventilator circuit 1402 may include a patient wye 1432, a sample tee 121 (e.g., gas sample tee 10 M/10 F), an inspiratory limb 213, a humidifier water chamber outlet 1434 (e.g., 22 M) on the wet side, a humidifier water chamber inlet 1436 (e.g., 22 M) on the dry side, a humidifier control unit 1438, a patient gas sample line 119 and filter 1440, a patient sample line inlet 728, an injector module electrical cable 1442, a delivery conduit 111, an injector module 107, a ventilator expiratory port 1444, and/or a ventilator inspiratory port 1446.

The conventional ventilator 205 may deliver breathing gas to the patient (not shown in FIG. 14A) via inspiratory limb 213 of the patient breathing circuit. For example, breathing gas may flow from the ventilator inspiratory port 1446 of the conventional ventilator 205. At injector module 107 the breathing gas may receive therapeutic gas from the therapeutic gas delivery system 100. A humidifier control unit 1438 may receive the breathing gas at the humidifier water chamber inlet 1436 (e.g., 22 M dry side) and discharge breathing gas at the humidifier water chamber outlet 1434 (e.g., 22 M wet side). In some aspects, the patient wye 1432 may be in fluid communication with both the inspiratory limb 213 and the expiratory limb 215 of the patient breathing circuit.

The conventional ventilator 205 may receive the expiration of the patient via expiratory limb 215 of patient breathing circuit. For example, expiration may flow from the patient wye 1432 through the expiratory limb 215 to the ventilator expiratory port 1444. The ventilator 205 may then deliver breathing gas through the ventilator inspiratory port 1446 to the inspiratory limb 213.

The sample line 119 may receive sample gas via the sample tee 121 in the inspiratory limb 213 of the breathing circuit. The sample gas may pass through a patient gas sample filter 1440 on the patient gas sample line 119. The patient gas sample filter 1440 may adjust the humidity of the sample gas, providing for more accurate testing of the sample gas. The sample gas may enter the sample gas subsystem at the sample line inlet 728.

FIG. 14B illustrates one example of an anesthesia circuit 1404. The anesthesia circuit 1404 may include a therapeutic gas delivery system 100, an injector module electrical cable 1442, a delivery conduit 111, inspiratory limb 213, one or more 22 M/15 F×22 M/15 F adapters 1448(a) and 1448 (b), a gas sample tee 121, a patient wye 1432, an absorber 1450, a ventilator expiratory port 1444, a ventilator 205, a bellows assembly 1452, a ventilator inspiratory port 1446, an injector module 107, a patient gas sample line 119 and filter 1440, and/or a patient sample line inlet 728.

The ventilator 205 may deliver breathing gas to the patient via an inspiratory limb 213 of the breathing circuit. For example, breathing gas may flow from the ventilator inspiratory port 1446 of the ventilator 205. At the injector module 107 the breathing gas may receive therapeutic gas from the therapeutic gas delivery system 100. The injector module may then be connected to a 22 M/15 F×22 M/15 F adapter 1448(b) to another portion of the inspiratory limb 213. The breathing gas may travel through the remainder of the inspiratory limb 213 and be delivered to the patient at the patient wye 1432.

The ventilator 205 may receive the expiration of the patient via the expiratory limb 215 of the breathing circuit. For example, expiration may travel from the patient wye 1432 through the expiratory limb 215 to the ventilator expiratory port 1444 of the ventilator 205.

A sample of the patient gas may be collected for testing in the gas analyzing subsystem 180. The sample gas may be received in the patient gas sample line 119 at the gas sample tee 121. The sample gas tee may be connected to the inspiratory limb 213 via a 22 M/15 F×22 M/15 F adapter 1448(a). The sample gas may be filtered before testing at the patient gas sample filter 1440. The sample gas may then be received into the gas analyzing subsystem 180 at the sample line inlet 728.

In some examples, fresh gas flow should be maintained at a rate greater than the patient's minute volume. In other words, recirculation of gases during use with the anesthesia device (e.g., if fresh gas flows are less than the patient's minute volume) should be avoided. For anesthesia systems with the inspiratory flow measurements at the inspiratory port of the absorber, the injector module may be placed upstream of the inspiratory flow sensor.

FIG. 14C illustrates one example of a spontaneous circuit 1406. The spontaneous circuit 1406 may include a therapeutic gas delivery system 100, an O₂ flowmeter 1454 (e.g., wall outlet or cylinder), O₂ tubing 1108, a 15 M×4.5 mm adapter 1456, one or more 22 M/15 F×22 M/15 F adapters 1448(a), 1448(b), and 1448(c), a first hose 1458 (e.g., 300 mm of 22 mm hose), an injector module 107, an injector module electrical cable 1442, a delivery conduit 111, a patient gas sample line inlet 728, a patient gas sample line 119 and filter 1440, a second hose 1460 (e.g., 1,200-1,600 mm of 22 mm hose), a gas sample tee 121, a cuff adapter 1462 (e.g., 22 mm ID×22 mm ID), and/or an aerosol mask 1464.

The O₂ flowmeter 1454 (e.g., wall outlet or cylinder) may deliver breathing gas to a patient via a hose system. The O₂ flowmeter 1454 (e.g., wall outlet or cylinder) may be connected to O₂ tubing 1108. The O₂ tubing may be connected to a first hose 1458 using a 14 M×4.5 mm adapter 1456 and a 22 M/15 F×22 M/15 F adapter 1448(a). The first hose 1458 may be connected to the injector module 107. At the injector module 107 the breathing gas may receive therapeutic gas from the therapeutic gas delivery system 100. The injector module 107 may then be connected to a second hose 1460 via a 22 M/15 F×22 M/15 F adapter 1448(b). The second hose 1460 may be connected to a sample tee 121. The sample tee may be connected to a patient gas sample line 119 and an aerosol mask 1464. The sample tee may be connected to the aerosol mask using a 22 M/15 F×22 M/15 F adapter 1448(c) and a cuff adapter 1462. Breathing gas may be delivered to the patient via the aerosol mask 1464.

Sample gas may be tested in the gas sample subsystem. Sample gas may flow from the gas sample tee 121 through the patient gas sample line 119 to a patient gas sample filter. The patient gas sample filter 1440 may filter the sample gas to prepare the gas for testing in the gas sample subsystem. The sample gas may enter the gas sample subsystem at the sample line inlet 728.

FIG. 14D illustrates one example of a nasal cannula circuit 1408. The nasal cannula circuit 1408 may include a therapeutic gas delivery system 100, an O₂ flowmeter 1454 (e.g., wall outlet or cylinder), O₂ tubing 1108, one or more 15 M×4.5 mm adapters 1456(a) and 1456(b), one or more 22 M/15 F×22 M/15 F adapters 1448(a), 1448(b), and 1448(c), a first hose 1458 (e.g., 300 mm of 22 mm hose), a second hose 1460 (e.g., 300 mm of 22 mm hose), an injector module 107, an injector module electrical cable 1442, a delivery conduit 111, a patient gas sample line 119 and filter 1440, a sample line inlet, an O₂ tubing sample tee 121, and/or a patient nasal cannula 1466.

The O₂ flowmeter 1454 (e.g., wall outlet or cylinder) may deliver breathing gas to a patient via a hose system. The breathing gas may travel from the O₂ flowmeter 1454 through O₂ tubing 1108. The O₂ tubing 1108 may then be connected to a first hose 1458 using a 15 M×4.5 mm adapter 1456(a) and a 22 M/15 F×22 M/15 F adapter 1448(a). The first hose may be connected to the injector module 107. At the injector module 107 the breathing gas may receive therapeutic gas from the therapeutic gas delivery system 100. The injector module 107 may then be connected to a second hose 1460 using a 22 M/15 F×22 M/15 F adapter 1448(b). The second hose may be connected to the gas sample tee 121 using a 15 M×4.5 mm adapter 1456(b) and a 22 M/15 F×22 M/15 F adapter 1448(c). The gas sample tee may be connected to the patient gas sample line 119 and a patient nasal cannula 1466. The patient nasal cannula may be used to deliver the breathing gas to the patient.

In some examples, the therapeutic gas delivery system 100 may be used with a nasal cannula to deliver therapeutic gas concentrations from 5-80 ppm and an oxygen flow rate as low as approximately 2 L/min. Conditioning of the oxygen prior to delivery though the injector module may ensure the most accurate flow measurement. In some examples, conditioning may be achieved by adding hose (e.g., approximately 300 mm of 22 mm hose) between the O₂ tubing and the injector module.

FIGS. 14E-14G illustrate one example of a High Frequency Jet Ventilation Circuit 1410. As illustrated in FIG. 14E, the High Frequency Jet Ventilation Circuit 1410 may include an high frequency jet ventilation system 1468, a therapeutic gas delivery system 100, an injector module electrical cable 1442, a delivery conduit 111, a patient sample line inlet 728 (e.g., patient gas sample line input connection), a first medical grade PVC tubing convenience pack 1479, a patient gas sample line 119 and filter 1440, one or more 15 M×4.5 mm ID adapters 1456(a) and 1456(b), one or more 22 M/15 F×22 M/15 F adapters 1448(a) and 1448(b), a cuff adapter 1462 (e.g., 22 mm ID×22 mm ID), an injector module 107, a second medical grade PVC tubing convenience pack 1481, a one-way valve 1472, a patient box 1474, a sample tee 121, a tapered adapter 1476, an endotracheal tube 1478, an adapter 1480, a humidifier 1112, a conventional ventilator 205, and/or a GAS OUT connection 1482.

In some examples, the high frequency jet ventilation system 1468 should be placed in standby before suctioning the patient. Otherwise, the system may over-deliver NO by as much as 30 ppm during this time period. In some examples, upon removing the suctioning catheter, the Enter button should be immediately pressured on the high frequency jet ventilation system to re-establish ventilation, which may limit the extent of over-delivery.

In some examples, a one-way valve may be placed between the injector module and the humidifier chamber to prevent water from backing up into the injector module. In an example, water may back up into the injector module when the high frequency jet ventilation system is in standby or turned off.

In some examples, the high frequency jet ventilation system 1468 may deliver higher pressure in the breathing circuit than conventional ventilators. Consequently, the user may ensure all circuit connections are secure and may use only parts configured for use with a high frequency jet ventilation system.

In some examples, a therapeutic gas delivery system convenience pack may include parts necessary to interface the therapeutic gas delivery system 100. For example, the therapeutic gas delivery system convenience pack may include a first medical grade PVC tubing convenience pack 1479, one or more 15 M×4.5 mm adapters 1456(a) and 1456(b), one or more 22 M/15 F×22 M/15 F adapters 1448(a) and 1448(b), a cuff adapter 1462 (e.g., 22 mm ID×22 mm ID), a second medical grade PVC tubing convenience pack 1481, a one-way valve 1472, and a sample tee 121. The first medical grade PVC tubing convenience pack 1479 may be cut into three pieces of medical grade PVC tubing, a first medical grade PVC tubing 1470 (e.g., cut to approximately 7 inches (17.8 cm)) and a second medical grade PVC tubing 1471 (e.g., cut to approximately 3 inches (7.6 cm)), and a third medical grade PVC tubing 1477. The second medical grade PVC tubing convenience pack may be cut into two pieces, both approximately 6 inches (15.24 cm) long, a fourth medical grade PVC tubing 1473 and a fifth medical grade PVC tubing 1475.

With respect to the High Frequency Jet Ventilation circuit 1410, FIG. 14 F illustrates an example of the injector module 107 connections. In some examples, the medical grade PVC tubing from the first medical grade PVC tubing convenience pack 1479 may be cut into three pieces of medical grade PVC tubing 1470, 1471, and 1477, as illustrated in FIG. 14E. One first medical grade PVC tubing 1470 may be approximately 7-inches (17.8 cm) long, the second medical grade PVC tubing 1471 may be approximately 3-inches (7.6 cm) long, and the third medical grade PVC tubing 1477 may be of sufficient length to connect the one-way valve 1472 to the high frequency jet ventilation system 1468. The first medical grade PVC tubing 1470, second medical grade PVC tubing 1471, and third medical grade PVC tubing 1477, the 15 M×4.5 mm adapters 1456(a) and 14456(b), the 15 M/22 F×15 M/22 F adapters 1448(a) and 1448(b), the cuff adapter 1462, the one-way valve 1472, and the injector module 107 may be attached, as illustrated in FIG. 14F. The medical grade PVC tubing may be removed from the Life Pulse GAS OUT connection 1482 on the front of the high frequency jet ventilation system 1468. The end of the removed tubing may be connected to the one-way valve 1472, as illustrated in FIG. 14F. The first medical grade PVC tubing convenience pack 1479 may be connected to the Life Pulse GAS OUT connection 1482 on the front of the high frequency jet ventilation system 1468.

With respect to the High Frequency Jet Ventilation circuit 1410, FIG. 14G illustrates an example of the patient gas sample line 119 connections. The fourth piece of medical grade PVC tubing 1473 and fifth piece of medical grade PVC tubing 1475 may be cut between the patient box 1474 and the tapered adapter 1476 at the midpoint (e.g., about 6 inches (15.2 cm) from the adapter 1480, as illustrated in FIG. 14G. The cut ends of the fourth medical grace PVC tubing 1473 and fifth medical grade PVC tubing 1475 may be connected to the gas sample tee 121. The patient gas sample line 119 and filter 1440 may be connected to the luer connector on the gas sample tee 121.

FIG. 14H illustrates one example of an High Frequency Oscillatory Ventilator unfiltered circuit 1412. The High Frequency Oscillatory Ventilator unfiltered circuit 1412 may include a therapeutic gas delivery system 100, a ventilator outlet 1484, a 3100A ventilator 1486 (e.g., top view), a valve control 1488, a limit valve control 1490, a sample port adapter 1492 (e.g., 90-degrees), a dump valve control 1494, a bias flow tube 1496, a humidifier outlet 1434, a humidifier inlet 1436, a cuff adapter 1462 (e.g., 22 mm ID×22 mm ID), a one-way valve 1472, a 22 M/15 F×22 M/15 F adapter 1448, an injector module 107, an injector module electrical cable 1442, a delivery conduit 111, a patient gas sample line 119 and filter 1440, and/or a patient gas sample line inlet 728.

In some instances, a therapeutic gas delivery system 3100A unfiltered convenience pack may include parts necessary to interface the therapeutic gas delivery system 100. For example, the therapeutic gas delivery system 3100A unfiltered convenience pack may include a sample port adapter 1492 (e.g., 90-degrees), a cuff adapter 1462 (e.g., 22 mm ID×22 mm ID), a one-way valve 1472, and/or a 22 M/15 F×22 M/15 F adapter 1448.

Breathing gas may be delivered to the patient by the 3100A ventilator 1486. The breathing gas may travel from the 3100A ventilator 1486 through the ventilator outlet 1484. The breathing gas may then flow through the injector module 107. At the injector module 107 the breathing gas may receive therapeutic gas from the therapeutic gas delivery system 100. The injector module 107 may be connected to a humidifier inlet 1436 by using a 22 M/15 F×22 M/15 F adapter 1448, a one-way valve 1472, and a cuff adapter 1462 (e.g., 22 mm ID×22 mm ID). The breathing gas may then flow through the humidifier outlet 1434 and into the bias flow tube 1496. The breathing gas then flows through a dump valve control 1494, a valve control 1488, and a limit valve control 1490 before being delivered to the patient.

Sample gas may be tested in the sample subsystem. A sample port adapter 1492 (e.g., 90-degrees) may provide a gas sample to the patient gas sample line 119. The gas sample may then be filtered by the patient gas sample filter 1440 before entering the sample subsystem at the sample line inlet 728.

FIGS. 14I-14J illustrate two examples of a dual-limb transport ventilator circuit 1414. For example, the dual-limb transport ventilator circuit 1414 is illustrated without an exhalation valve in FIG. 14I and with an exhalation valve in FIG. 14J.

The dual-limb transport ventilator circuit 1414 may include a therapeutic gas delivery system 100, a ventilator 205, a ventilator expiratory port 1444 (FIG. 14I) or a ventilator expiratory valve 1498 (FIG. 14J), an expiratory limb 215, a patient wye 1432, a gas sample tee 121, one or more 22 M/15 F×22 M/15 F adapters 1448(a), 1448(b), an inspiratory limb 213, an injector module 107, a ventilator inspiratory port 1446, an injector module electrical cable 1442, a delivery conduit 111, a patient gas sample line 119 and filter 1440, and/or a patient gas sample line inlet 728.

In some examples, depending on the ventilator, patient circuit, or tubing being used, different adapters may be used based on the set-up of the dual-limb transport ventilator circuit 1414.

In FIG. 14I, the ventilator 205 may deliver breathing gas to a patient through the inspiratory limb 213. The breathing gas may exit the ventilator 205 at the ventilator inspiratory port 1446 and flow into the inspiratory limb 213. The gas may then flow through the injector module 107. At the injector module 107 the breathing gas may receive therapeutic gas from the therapeutic gas delivery system 100. The injector module is connected back to the inspiratory limb using a 22 M/15 F×22 M/15 F adapter 1448(a). The breathing gas flows through the inspiratory limb 213 where the inspiratory limb 213 is connected to a sample tee 121 using a 22 M/15 F×22 M/15 F adapter 1448(b). The breathing gas is then delivered to the patient at the patient wye 1432.

The ventilator 205 may receive expiration through the expiratory limb 215. The expiratory limb 215 is connected to the patient wye 1432 on one end and the ventilator expiratory port 1444 on the other end. The expiration flows from the patient wye 1432 through the expiratory limb 215 and into the ventilator at the ventilator expiratory port 1444.

A gas sample may be tested in the gas sample subsystem. The gas sample is taken from the sample tee 121 into the patient gas sample line 119. The sample gas may be filtered by the patient gas sample filter 1440 before entering the gas sample subsystem at the sample line inlet 728.

In FIG. 14J, the ventilator 205 may deliver breathing gas to a patient through the inspiratory limb 213. The breathing gas may exit the ventilator 205 at the ventilator inspiratory port 1446 and flow into the inspiratory limb 213. The gas may then flow through the injector module 107. At the injector module 107 the breathing gas may receive therapeutic gas from the therapeutic gas delivery system 100. The injector module is connected back to the inspiratory limb using a 22 M/15 F×22 M/15 F adapter 1448(a). The breathing gas flows through the inspiratory limb 213 where the inspiratory limb 213 is connected to a sample tee 121 using a 22 M/15 F×22 M/15 F adapter 1448(b). The breathing gas is then delivered to the patient at the patient wye 1432.

The ventilator 205 may receive expiration through the expiratory limb 215. The expiratory limb 215 is connected to the patient wye 1432 on one end and the ventilator expiratory valve 1498 on the other end. The expiration from the patient wye 1432 flows into the ventilator 205 at the ventilator expiratory valve 1498.

A gas sample may be tested in the sample subsystem. The gas sample is taken from the sample tee 121 into the patient gas sample line 119. The sample gas may be filtered by the patient gas sample filter 1440 before entering the gas sample subsystem at the sample line inlet 728.

FIG. 14K illustrates one example of a single-limb transport ventilator circuit 1416. The single-limb transport ventilator circuit 1416 may include a therapeutic gas delivery system 100, a ventilator 205, a positive end expiratory pressure (“PEEP”) valve 1403, a patient/exhalation valve 1401, a gas sample tee 121, one or more 22 M/15 F×22 M/15 F adapters 1448(a), 1448(b), an inspiratory limb 213, an injector module 107, a ventilator inspiratory port 1446, an injector module electrical cable 1442, a delivery conduit 111, a patient gas sample line 119 and filter 1440, and/or a patient gas sample line inlet 728.

Breathing gas may be delivered to the patient by the ventilator 205. The breathing gas may exit the ventilator 205 at the ventilator inspiratory port 1446 and flow through the inspiratory limb 213. The injector module 107 may be placed along the inspiratory limb 213. At the injector module 107 the breathing gas may receive therapeutic gas from the therapeutic gas delivery system 100. The injector module may be reconnected to the inspiratory limb 213 by a 22 M/15 F×22 M/15 F adapter 1448(a). The inspiratory limb 213 may be connected to a sample tee 121 via a 22 M/15 F×22 M/15 F adapter 1448(b). The inspiratory limb 213 then connects delivers gas to the patient via the patient/exhalation valve 1401. A PEEP valve 1403 may be connected to the end of the inspiratory limb 213 before the breathing gas enters the patient/exhalation valve 1401.

A gas sample may be tested in the sample subsystem. The gas sample is taken from the sample tee 121 into the patient gas sample line 119. The sample gas may be filtered by the patient gas sample filter 1440 before entering the gas sample subsystem at the sample line inlet 728.

FIG. 14L illustrates one example of an High Frequency Oscillatory Ventilator filtered circuit 1418. The High Frequency Oscillatory Ventilator filtered circuit 1418 may include an therapeutic gas delivery system 100, a ventilator outlet 1484, a 3100A ventilator 1486 (e.g., top view), a valve control 1488, a dump valve control 1494, one or more filters 1407(a) and 1407(b), a sample port adapter 1492 (e.g., 90-degrees), a bias flow tube 1496, a humidifier outlet 1434, a humidifier inlet 1436, a limit valve control 1490, a one-way valve 1472, one or more 22 M OD×10 mm OD adapters 1409(a) and 1409(b), an injector module 107, a cuff adapter 1462 (e.g., 22 mm ID×22 mm ID), an injector module electrical cable 1442, a delivery conduit 111, a patient gas sample line 119 and filter 1440, and/or a patient gas sample line inlet 728.

In FIG. 14L, breathing gas may be delivered to a patient using a 3100A ventilator 1486. The breathing gas may exit the 3100A ventilator 1486 at a ventilator outlet 1484. The breathing gas may then enter the injector module 107 through a 22 M OD×10 mm OD adapter 1409(a) and a cuff adapter 1462 (e.g., 22 mm ID×22 mm ID). At the injector module 107 the breathing gas may receive therapeutic gas from the therapeutic gas delivery system 100. The breathing gas then exits the injector module 107 and passes through a 22 M OD×10 mm OD adapter 1409(b) and a one-way valve 1472. The breathing gas then may travel through a limit valve control 1490, followed by a filter 1407(a). The breathing gas then enters a humidifier at a humidifier inlet 1436. The breathing gas may exit the humidifier at the humidifier outlet 1434 into a bias flow tube 1496. The breathing gas then passes through a valve control 1488, a dump valve control 1494, and a filter 1407(b) before being delivered to the patient.

Sample gas may be tested in the sample subsystem. Sample gas is taken from the breathing circuit by a sample port adapter 1492 (90-degrees). The sample gas then may flow through a patient gas sample line 119 and through a patient gas sample filter 1440 before entering the sample subsystem at the sample line inlet 728.

In some instances, a therapeutic gas delivery system 3100A filtered convenience pack may include parts necessary to interface the therapeutic gas delivery system. For example, the therapeutic gas delivery system 3100A unfiltered convenience pack may include a sample port adapter 1492 (e.g., 90-degrees), a one-way valve 1472, an adapter 1409 (e.g., 22 mm OD×10 mm OD), and/or a cuff adapter 1462 (e.g., 22 mm ID×22 mm ID).

FIG. 14M illustrates one example of an Infant Flow Circuit 1420. The Infant Flow Circuit 1420 may include a therapeutic gas delivery system 100, a patient gas sample line inlet 728, a delivery conduit 111, an injector module electrical cable 1442, a patient gas sample line 119 and filter 1440, an injector module 107, a humidifier 1112, a heated delivery circuit 1411, a temperature probe 1118, a gas sample tee 121 (e.g., 10 M/10 F), an infant flow generator 1413, an infant flow ventilation system 1415, an abdominal respiratory sensor 1417, and/or a transducer interface 1419.

Breathing gas may be delivered to a patient through the use of an infant flow ventilation system 1415. The breathing gas exits the infant flow ventilation system 1415 and flows through tubing to the injector module 107. At the injector module 107 the breathing gas may receive therapeutic gas from the therapeutic gas delivery system 100. The breathing gas then may exit the injector module 107 and enter a humidifier 1112. After exiting the humidifier, the breathing gas may travel through a heated delivery circuit 1411 and through a sample tee 121. A temperature probe 1118 may be used to measure the temperature of the breathing gas while it is in the heated delivery circuit 1411. After passing through the sample tee 121, the breathing gas may be delivered to a patient through the infant flow generator 1413. An abdominal respiratory sensor 1417 and transducer interface 1419 may be connected to the infant flow ventilation system 1415.

A gas sample may be tested in the sample subsystem. The gas sample is taken from the sample tee 121 into the patient gas sample line 119. The sample gas may be filtered by the patient gas sample filter 1440 before entering the gas sample subsystem at the sample line inlet 728.

In some examples, the therapeutic gas delivery system may add therapeutic gas flow to the breathing circuit flow in proportion to the NO setting (e.g., up to approximately 3.7% at 80 ppm). Moreover, the therapeutic gas delivery system may subtract gas from the breathing circuit via gas sampling at a nominal flow rate of approximately 0.23 L/min. These effects may change the flow to the nasal adapter, which may impact the continuous positive airway pressure level established by the flow settings. In some examples, the maximum flow error is approximately 11% at 2 L/min, which may be within the accuracy of the flowmeter specification (+/−15%). After the NO setting is changed, the continuous positive airway pressure level may be checked on the Infant Flow ventilation system front panel display so that the level may be adjusted as necessary.

FIG. 14N illustrates one example of a Precision Flow Circuit 1422. The Precision Flow Circuit 1422 may include a therapeutic gas delivery system 100, a patient gas sample line inlet 728, a delivery conduit 111, an injector module electrical cable 1442, a patient gas sample line 119 and filter 1440, a patient cannula 1466, a sample tee 121, a patient gas delivery tube 1425, a 22 M/15 F×22 M/15 F adapter 1448, an injector module 107, a 22 F×15 M adapter 1421, and/or a precision flow unit 1423.

In FIG. 14N, breathing gas may be delivered to a patient using a precision flow unit 1423. Breathing gas may exit the precision flow unit 1423 and enter tubing. A 22 F×15 M adapter may connect the tubing to the injector module 107. At the injector module 107 the breathing gas may receive therapeutic gas from the therapeutic gas delivery system 100. After exiting the injector module, the breathing gas may flow through a 22 M/15 F×22 M/15 F adapter 1448 and into tubing. The breathing gas may travel through the tubing back into the precision flow unit 1423. The breathing gas may then exit the precision flow unit 1423 and enter a patient gas delivery tube 1425. The patient gas delivery tube 1425 may be connected to a sample tee 121. Once the breathing gas passes through the sample tee 121 it may be delivered to the patient via a patient cannula 1466.

A gas sample may be tested in the sample subsystem. The gas sample is taken from the sample tee 121 into the patient gas sample line 119. The sample gas may be filtered by the patient gas sample filter 1440 before entering the gas sample subsystem at the sample line inlet 728.

In some examples, the therapeutic gas delivery system may add therapeutic gas flow to the breathing circuit flow in proportion to the NO setting (e.g., up to approximately 1.6% at 80 ppm). Moreover, the therapeutic gas delivery system 100 may subtract gas from the breathing circuit via gas sampling at a nominal flow rate of approximately 230 mL/min. These effects may impact the delivered gas flow rate when the precision flow circuit is used. After the NO setting is changed, the delivered gas flow rate may be checked and the gas source flow rate may be adjusted as necessary.

FIG. 14O illustrates one example of a Continuous Positive Airway Pressure System Circuit 1424. The Continuous Positive Airway Pressure System Circuit 1424 may include a therapeutic gas delivery system 100, a patient gas sample line inlet 728, a delivery conduit 111, an injector module electrical cable 1442, a patient gas sample line 119 and filter 1440, a pressure manifold 1447, an injector module 107, a humidifier 1112 (e.g., MR850), a continuous positive airway pressure generator 1427, a nasal prong infant interface 1429, a gas sample tee 121 (e.g., 10 M/10 F), a breathing circuit 209, a temperature probe 1118, O₂ tubing 1108, and/or an O₂ source 1431.

In FIG. 14O, breathing gas may be delivered to a patient by an O₂ source 1431. The breathing gas may exit the O₂ source 1431 into O₂ tubing 1108. The breathing gas may then pass through a pressure manifold 1447 and into the injector module 107. At the injector module 107 the breathing gas may receive therapeutic gas from the therapeutic gas delivery system 100. The breathing gas may then exit the injector module, travel through tubing, and enter a humidifier 1112. After exiting the humidifier 1112, the gas may travel through tubing to a breathing circuit 209. A temperature probe 1118 may be connected to the breathing circuit 209 to measure the temperature of the breathing gas. The breathing gas may then pass through a sample tee 121 and be delivered to a patient via a nasal prong infant interface. Patient expiration may leave the patient and flow to a continuous positive airway pressure generator 1427.

A gas sample may be tested in the sample subsystem. The gas sample is taken from the sample tee 121 into the patient gas sample line 119. The sample gas may be filtered by the patient gas sample filter 1440 before entering the gas sample subsystem at the sample line inlet 728.

FIG. 14P illustrates one example of a High Flow Breathing Circuit 1426. The High Flow Breathing Circuit 1426 may include a therapeutic gas delivery system 100, a patient gas sample line inlet 728, a patient gas sample line 119 and filter 1440, a delivery conduit 111, an injector module electrical cable 1442, an injector module 107, a pressure relief manifold 1433, O₂ tubing 1108, an O₂ source 1431, a temperature probe 1118, a nasal cannula 1466, an adapter 1435, a breathing circuit 209, a sample tee 121, and/or a humidifier 1112. In some examples, the adapter 1435 is configured for high flow. The adapter 1435 may further be configured for pediatric use.

In FIG. 14P, breathing gas may be delivered to a patient by an O₂ source 1431. The breathing gas may exit the O₂ source 1431 and travel through O₂ tubing 1108 to a pressure relief manifold 1433. After passing through the pressure relief manifold, the breathing gas may enter the injector module 107. At the injector module 107 the breathing gas may receive therapeutic gas from the therapeutic gas delivery system 100. The breathing gas may then exit the injector module 107 and enter a humidifier 1112. After exiting the humidifier 1112, the breathing gas may flow through a breathing circuit to an adapter 1435 and be delivered to the patient via a nasal cannula 1466. A temperature probe 1118 may be connected to the breathing circuit 209 to measure the temperature of the breathing gas. A sample tee 121 may be connected to the breathing circuit 209.

A gas sample may be tested in the sample subsystem. The gas sample is taken from the sample tee 121 into the patient gas sample line 119. The sample gas may be filtered by the patient gas sample filter 1440 before entering the gas sample subsystem at the sample line inlet 728.

FIG. 14Q illustrates one example of a High Flow Infant Circuit Nasal Cannula Circuit 1428. The High Flow Infant Circuit Nasal Cannula Circuit 1428 may include a therapeutic gas delivery system 100, a patient gas sample line inlet, a patient gas sample line 119 and filter 1440, a delivery conduit 111, an injector module electrical cable 1442, an injector module 107, a pressure relief manifold 1433, O₂ tubing 1108, an O₂ source 1431, a nasal cannula 1466, a gas sample tee 121 (e.g., 10 M/10 F), a temperature probe 1118, a breathing circuit 209, and/or a humidifier 1112 (e.g., MR850).

In FIG. 14Q, breathing gas may be delivered to a patient through the use of an O₂ source 1431. Breathing gas may exit the O₂ source 1431 and flow through O₂ tubing 1108. The breathing gas may then flow through a pressure relief manifold 1433 and into the injector module 107. At the injector module 107 the breathing gas may receive therapeutic gas from the therapeutic gas delivery system 100. After passing through the injector module 107, the breathing gas may enter a humidifier 1112. After the breathing gas is humidified, the breathing gas may exit the humidifier 1112 and enter into a breathing circuit 209. A temperature probe 1118 may be connected to the breathing circuit 209, to measure the temperature of the breathing gas. A sample tee 121 may be connected to the breathing circuit 209. The breathing gas may pass through the sample tee 121 and travel through the remainder of the breathing circuit 209. The breathing gas may be delivered to the patient via a nasal cannula 1466.

A gas sample may be tested in the sample subsystem. The gas sample is taken from the sample tee 121 into the patient gas sample line 119. The sample gas may be filtered by the patient gas sample filter 1440 before entering the gas sample subsystem at the sample line inlet 728.

FIG. 14R illustrates one example of a High Flow Nasal Cannula Oxygen Therapy Circuit 1430. The High Flow Nasal Cannula Oxygen Therapy Circuit 1430 may include a therapeutic gas delivery system 100, a patient gas sample line inlet 728, a delivery conduit 111, an injector module electrical cable 1442, a patient gas sample line 119 and filter 1440, a cannula 1466, a high flow adapter 1435, an inspiratory limb 213, an O₂ inlet port 1437, a filter cover 1439, a high flow nasal cannula oxygen therapy device 1441 a water bag 1443, one or more 15 M×4.5 mm adapters 1456(a) and 1456(b), two or more 22 M/15 F×22 M/15 F adapters 1448(a) and 1448(b), an injector module 107, a 22 F×15 M adapter 1421, O₂ tubing 1108, a flowmeter 1454, a gas sample tee 121, and/or an air/O₂ blender 1445.

In FIG. 14R, breathing gas may be delivered to a patient starting at an air/O₂ blender 1445. The breathing gas may exit an air/O₂ blender 1445 and enter into a flowmeter 1454. After exiting the flowmeter 1454, the breathing a may flow through O₂ tubing 1108. The O₂ tubing 108 may be connected to the injector module 107 by using a 15 M×4.5 mm adapter 1456(a), a 22 M/15 F×22 M/15 F adapter 1448(a), and a 22 F×15 M adapter 1421. At the injector module 107 the breathing gas may receive therapeutic gas from the therapeutic gas delivery system 100. After passing through the injector module 107, the breathing gas may pass through a 22 M/15 F×22 M/15 F adapter 1448(b) and a 15 M×4.5 mm adapter 1456(b) which connect the injector module 107 to O₂ tubing 1108. The breathing gas may then flow through the O₂ tubing 1108 into an O₂ inlet port 1437 before entering into a high flow nasal cannula oxygen therapy device 1441. A water bag 1443 may be used in conjunction with the high flow nasal cannula oxygen therapy device 1441. A filter cover 1439 may connected to the high flow nasal cannula oxygen therapy device 1441. The breathing gas may then exit the high flow nasal cannula oxygen therapy device 1441 into an inspiratory limb 213. After flowing through the inspiratory limb 213, the breathing gas may pass through an adapter 1435 and a sample tee 121. The breathing gas may then be delivered to the patient via a cannula 1466.

A gas sample may be tested in the sample subsystem. The gas sample is taken from the sample tee 121 into the patient gas sample line 119. The sample gas may be filtered by the patient gas sample filter 1440 before entering the gas sample subsystem at the sample line inlet 728.

The high flow nasal cannula oxygen therapy device 1441 may include a flow generator internal to the device. The flow generator may create high flow rates which may enter through the filter on the bac of the device before passing through the enclosed humidifier. Adding oxygen through the oxygen inlet port may allow for supplementing the FiO₂ being delivered to the patient but might not account for all of the gas flow going to the patient. To prevent room air from entering through the filter cover and diluting the nitric oxide level coming from the Injector Module an air/O₂ blender may be used to drive the flow through the Injector Module and into the oxygen inlet hose. By matching the flow rate on the air/O₂ blender with the flow setting on the high flow nasal cannula oxygen therapy device 1441, the monitored dose may equal the set dose. In some examples the failure to match the air/O₂ blender flow rate with the high flow nasal cannula oxygen therapy device 1441 flow setting may result in therapeutic gas levels delivered to the patient that are lower than the set dose.

FIGS. 15A-15H illustrate one example of a method 1500 to load and/or rep ace a therapeutic gas source 116(a), 116(b) in the therapeutic gas delivery system 100. As previously discussed, each therapeutic gas source 116(a), 116(b) may be a compressed gas cylinder 714(a), 714(b). Each compressed gas cylinder 714(a), 714(b) may be received within a respective bay 716(a), 716(b) of the therapeutic gas delivery system 100. In some aspects, each bay 716(a), 716(b) may be operably enclosed by a respective door 718(a), 718(b), which may be opened to access the bay 716 and closed to enclose the bay 716. In some aspects, the cylinder valve area 1502(a), 1502(b) of the compressed gas cylinder 714(a), 714(b) may be removably coupled to the respective cylinder manifold 1506(a), 1506(b) of the therapeutic gas delivery system 100. For example, a lever 720(a), 720(b) may be actuated to removably couple (or decouple) the compressed gas cylinder 714 to the therapeutic gas delivery system 100. For example, the lever 720(a), 720(b) may be actuated by raising or lowering the lever 720(a), 720(b).

Each compressed gas cylinder 714(a), 714(b) may contain therapeutic gas (e.g., NO gas). In some examples before loading a new and/or full compressed gas cylinder 714 (e.g., new cylinder, full cylinder) into a respective bay 716, one or more aspects (e.g., cylinder concentration, expiration date) of the compressed gas cylinder 714 may be verified to ensure that the compressed gas cylinder 714 is adequate to complete treatment of the patient.

In some embodiment's, to ensure uninterrupted delivery of therapeutic gas to the patient, the therapeutic gas delivery system 100 may include two compressed gas cylinders 714(a), 714(b) or, in some cases, more than two compressed cylinders. In some aspects, a first compressed gas cylinder 714 (e.g., 714(a)) may deliver therapeutic gas while a second compressed gas cylinder 714 (e.g., 714(b) serves as a backup. The therapeutic gas delivery system 100 may include an automatic cylinder switchover function for continuous delivery of therapeutic gas. The automatic switchover function may changeover the delivery of therapeutic gas from a partially depleted compressed gas cylinder to a full compressed gas cylinder. In an example, a partially depleted compressed gas cylinder has a pressure of 210 psig or less. For example, when the first cylinder (e.g., compressed gas cylinder 714(a)) that had been delivering therapeutic gas (e.g., delivering cylinder) becomes partially depleted (e.g. 210 psig or less), the therapeutic gas delivery system 100 may automatically switch over to deliver therapeutic gas from the second cylinder 716(b). Then, when the second cylinder is delivering therapeutic gas, the first partially depleted cylinder may be removed and replaced by removing the first cylinder from the first bay 716(a) and loading a new replacement cylinder into the first bay 716(a). Then, when the second cylinder becomes partially depleted, the therapeutic gas delivery system 100 may automatically switch over to deliver therapeutic gas from the new replacement cylinder in the first bay 716(a). In this manner, the automatic cylinder switchover function may enable continuous delivery from alternate bays 716(a). 716(b), while providing time to remove and replace a partially depleted cylinder such that the therapeutic gas delivery system 100 may continuously deliver therapeutic gas.

Although FIGS. 15A-15H illustrate loading and/or replacing compressed gas cylinder 714(a) in bay 716(a) of the therapeutic gas delivery system 100, the same steps may be applied to load and/or replace compressed gas cylinder 714(b) in bay 716(b). Loading and/or replacing compressed gas cylinder 714(a) may involve one or more of the steps described below.

As illustrated in FIG. 15A, the door 718(a) of the cylinder bay 716(a) may be opened, such that the bay 716(a) is accessible. A new compressed gas cylinder (not shown in FIG. 15A) may be obtained and be kept on hand near the therapeutic gas delivery system 100. As illustrated in FIG. 158 the lever 720(a) may be raised to disengage/release the compressed gas cylinder 714(a) from the therapeutic gas delivery system 100. In some examples, if a compressed gas cylinder 714(a) is not present in the bay 716(a), raising the lever 720(a) may reveal the cylinder manifold 1506(a) (e.g., 1202(a)) in the bay 716(a). In other examples, if compressed gas cylinder 714(a) is present (e.g., a depleted cylinder) in the bay 716(a), actuating the lever 720(a) may disengage the compressed gas cylinder 714(a) from the therapeutic gas delivery system 100.

After disengaging the compressed gas cylinder 714(a), the compressed gas cylinder 714(a) (e.g., partially depleted cylinder) may be removed from the bay 716(a), as illustrated in FIG. 15C. In some examples, removing the compressed gas cylinder 714(a) from the bay 716(a) may reveal the cylinder manifold 1506(a) on the cylinder manifold interface. A new or full compressed gas cylinder 714(a), which contains therapeutic gas (e.g., NO gas), may be inserted into the bay 716(a), as illustrated in FIG. 15D. In some examples, a new compressed gas cylinder 714(a) may include shrink wrap over the cylinder valve area 1502(a) (e.g., therapeutic gas source valve 117(a)). The shrink wrap may be removed from the cylinder valve area 1502(a) before inserting the new compressed gas cylinder 714(a) into the bay 716(a).

The compressed gas cylinder 714(a) may include one or more keyways 1504, as illustrated for example in FIG. 15E. The cylinder manifold 1506 (e.g., 1202(a), 1202(b)) may include one or more key alignment indicators 1508 and/or one or more keys 1510, as illustrated for example in FIG. 15F. As the new compressed gas cylinder 714(a) is inserted into the bay 716(a), the keyways 1504 on the compressed gas cylinder 714(a) (as illustrated in FIG. 15E) may be aligned with the corresponding keys 1510 on the cylinder manifold 1506 of the therapeutic gas delivery system 100 (as illustrated in FIG. 15F). In some aspects, the new compressed gas cylinder 714(a) may be advanced (e.g., slid) to the beck of the bay 716(a) until it rests against the cylinder manifold 1508.

As illustrated in FIG. 15G, the lever 720(a) may be lowered within the bay 716(a), to removably couple the new compressed gas cylinder 714(a) to the therapeutic gas delivery system 100. In some examples, the lever 720(a) may be actuated until it comes to rest against the body of the compressed gas cylinder 714(a). In some examples, an automated cylinder purge may occur when the lever 720(a) is lowered (e.g., when the compressed gas cylinder 714(a) is removably coupled to the therapeutic gas delivery system 100). As illustrated in FIG. 14H, the door 718(a) may be closed, thereby enclosing the compressed gas cylinder 714(a) in the bay 716(a).

In some examples, the therapeutic gas delivery system 100 (e.g., EVOLVE delivery device) may respond to an operator error (e.g., operator accidentally changed the wrong cylinder). For example, if one compressed gas cylinder 714 (e.g., 714(a), 714(b)) is already partially depleted (e.g., 210 psig or less) and the non-depleted cylinder 714 (e.g., 714(a), 714(b)) is accidentally removed from the therapeutic gas delivery system 100, then the therapeutic gas delivery system 100 will not switchover to the compressed gas cylinder 714 that is partially depleted. In some examples, the therapeutic gas delivery system 100 may stall for approximately 60 seconds and/or provide one or more alarms (e.g., visual, audible) if a replacement compressed gas cylinder 714 (e.g., new cylinder, full cylinder) is not loaded (e.g., a depleted compressed gas cylinder 714 is loaded).

FIG. 16 shows an example of computing system 1600, which can be for example any computing device making up various parts of the gas delivery system, or any component thereof in which the components of the system are in communication with each other using connection 1605. Connection 1605 can be a physical connection via a bus, or a direct connection into processor 1610, such as in a chipset architecture. Connection 1605 can also be a virtual connection, networked connection, or logical connection.

In some embodiments, computing system 1600 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example system 1600 includes at least one processing unit (CPU or processor) 1610 and connection 1605 that couples various system components including system memory 1615, such as read-only memory (ROM) 1620 and random-access memory (RAM) 1625 to processor 1610. Computing system 1600 can include a cache of high-speed memory 1612 connected directly with, in close proximity to, or integrated as part of processor 1610.

Processor 1610 can include any general purpose processor and a hardware service or software service, such as services 1632, 1634, and 1636 stored in storage device 1630, configured to control processor 1610 as well as a special-purpose processor where software instructions are incorporated into the actual processor design. Processor 1610 may essentially be a completely self-contained computing system, containing multiple cores or processors, a bus, memory controller, cache, etc. A multi-core processor may be symmetric or asymmetric.

To enable user interaction, computing system 1600 includes an input device 1645, which can represent any number of input mechanisms, such as a microphone for speech, a touch-sensitive screen for gesture or graphical input, keyboard, mouse, motion input, speech, etc. Computing system 1600 can also include output device 1635, which can be one or more of a number of output mechanisms known to those of skill in the art. In some instances, multimodal systems can enable a user to provide multiple types of input/output to communicate with computing system 1600. Computing system 1600 can include communications interface 1640, which can generally govern and manage the user input and system output. There is no restriction on operating on any particular hardware arrangement, and therefore the basic features here may easily be substituted for improved hardware or firmware arrangements as they are developed.

Storage device 1630 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 1630 can include software services, servers, services, etc., that when the code that defines such software is executed by the processor 1610, it causes the system to perform a function. In some embodiments, a hardware service that performs a particular function can include the software component stored in a computer-readable medium in connection with the necessary hardware components, such as processor 1610, connection 1605, output device 1635, etc., to carry out the function.

For clarity of explanation, in some instances, the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program or a collection of programs that carry out a specific function. In some embodiments, a service can be considered a server. The memory can be a non-transitory computer-readable medium.

In some embodiments, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer-readable media. Such instructions can comprise, for example, instructions and data which cause or otherwise configure a general purpose computer, special purpose computer, or special purpose processing device to perform a certain function or group of functions. Portions of computer resources used can be accessible over a network. The executable computer instructions may be, for example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, USB devices provided with non-volatile memory, networked storage devices, and so on.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

It is to be understood that the invention is not limited to the details of construction or process steps set forth in the above description. The invention is capable of other embodiments and of being practiced or being carried out in various ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

### Exemplary Embodiments

The following is a list of non-limiting exemplary embodiments, and may include combinations thereof.

Embodiment 1: A therapeutic gas delivery system comprising: at least one gas supply subsystem comprising: a gas source coupling configured to receive a therapeutic gas source and form a fluid flow connection with the therapeutic gas source; a therapeutic gas delivery system controller comprising a CPU; one or more display(s) configured to be in communication over a communication path with the therapeutic gas delivery system controller, wherein the display is configured to display a graphical, illustrative, or numerical indicator of one or more flow or system parameters.

Embodiment 2: The therapeutic gas delivery system of claim 1, wherein the display is further configured to display a visual alarm.

Embodiment 3: The therapeutic gas delivery system of claim 2, wherein the therapeutic gas delivery system controller is configured to produce an audible alarm.

Embodiment 4: The therapeutic gas delivery system of claim 1, wherein the display is configured to present a bar graph, a chart, or a numerical display of a value of the one or more flow or system parameters.

Embodiment 5: The therapeutic gas delivery system of claim 1, wherein the display is configured to present a graphic of the therapeutic gas delivery system comprising indicators for flow of the therapeutic gas through the system.

Embodiment 6: The therapeutic gas delivery system of claim 5, wherein the graphic of the therapeutic gas delivery system further comprises indicators for a status of the therapeutic gas source, a sample line, an injector module, expiratory tubing, an electronically controlled gas blending device, or combinations thereof.

Embodiment 7: The therapeutic gas delivery system of claim 1, the therapeutic gas is nitric oxide.

Embodiment 8: The therapeutic gas delivery system of claim 7, wherein the display is configured to present the concentration and/or flow rate of nitric oxide and breathing gas through the system.

Embodiment 9: The therapeutic gas delivery system of claim 1, further comprising an electrical power supply connection, an internal battery, and a power button, wherein the power button includes a power indicator that visually indicates an electrical power supply status of the electrical power supply connection and a battery charge status of the internal battery.

Embodiment 10: The therapeutic gas delivery system of claim 9, wherein one or more system parameters includes an electrical power supply status and a battery charge status.

Embodiment 11: The therapeutic gas delivery system of claim 1, further comprising an alarm silence button, wherein the alarm silence button includes an alarm indicator that visually indicates the status of one or more alarms of the therapeutic gas delivery system.

Embodiment 12: The therapeutic gas delivery system of claim 1, further comprising a global status indicator, wherein the global status indicator visually indicates one or more global statuses of the therapeutic gas delivery system.

Embodiment 13: The therapeutic gas delivery system of claim 12, wherein one or more of the global statuses include a delivery status and an alarm status.

Embodiment 14: The therapeutic gas delivery system of claim 1, further comprising an injector module outlet and a corresponding port indicator, wherein the port indicator visually indicates a delivery status of the injector module outlet.

Embodiment 15: The therapeutic gas delivery system of claim 1, further comprising a secondary delivery subsystem outlet and a corresponding port indicator, wherein the port indicator visually indicates a delivery status of the secondary delivery subsystem outlet.

Embodiment 16: The therapeutic gas delivery system of claim 1, wherein the therapeutic gas source includes a first compressed gas cylinder, wherein the one or more flow or system parameters includes a first cylinder status, wherein the first cylinder status visually indicates whether the first compressed gas cylinder is delivering therapeutic gas or is idle, wherein the first cylinder status includes a percentage of available therapeutic gas in the first compressed gas cylinder.

Embodiment 17: The therapeutic gas delivery system of claim 16, wherein the first cylinder status visually indicates if the first compressed gas cylinder is in a low priority alarm or a high priority alarm.

Embodiment 18: The therapeutic gas delivery system of claim 16, wherein the therapeutic gas source includes a second compressed gas cylinder, wherein the one or more flow or system parameters includes a second cylinder status, wherein the second cylinder status visually indicates whether the second compressed gas cylinder is delivering therapeutic gas or is idle, wherein the second cylinder status includes a percentage of available therapeutic gas in the second compressed gas cylinder.

Embodiment 19: The therapeutic gas delivery system of claim 18, wherein the second cylinder status visually indicates if the second compressed gas cylinder is in a low priority alarm or a high priority alarm.

Embodiment 20: The therapeutic gas delivery system of claim 1, wherein the one or more flow or system parameters includes at least one of a left cylinder status, right cylinder status, a patient line status, a sample gas sample line status, a delivery conduit status, an injector module status, a patient gas sample line status, and a breathing circuit expiratory limb status.

Embodiment 21: The therapeutic gas delivery system of claim 1, wherein the one or more flow or system parameters includes at least one of a secondary delivery subsystem status and dose.

Embodiment 22: The therapeutic gas delivery system of claim 1, wherein at least one gas supply system includes a secondary delivery subsystem, wherein the one or more displays is configured to display the delivery of therapeutic gas through the secondary delivery subsystem.

Embodiment 23: The therapeutic gas delivery system of claim 22, further comprising a breathing circuit, wherein the one or more displays is configured to display the delivery of therapeutic gas to the breathing circuit.

Embodiment 24: The therapeutic gas delivery system of claim 23, wherein the breathing circuit is a t-piece resuscitator circuit, an infant resuscitator circuit, or an infant t-piece resuscitator circuit.

Embodiment 25: The therapeutic gas delivery system of claim 1, wherein at least one gas supply system includes a therapeutic gas delivery system, wherein the one or more displays is configured to display the delivery of therapeutic gas through the therapeutic gas delivery system.

Embodiment 26: The therapeutic gas delivery system of claim 25, further comprising a breathing circuit, wherein the one or more displays is configured to display the delivery of therapeutic gas to the breathing circuit.

Embodiment 27: The therapeutic gas delivery system of claim 26, wherein the breathing circuit is a conventional ventilator circuit, an anesthesia circuit, a spontaneous circuit, a nasal cannula circuit, a high frequency jet ventilation circuit, a high frequency oscillatory ventilator unfiltered circuit, a dual-limb transport ventilator circuit, or a single-limb transport ventilator circuit.

Embodiment 28: The therapeutic gas delivery system of claim 26, wherein the breathing circuit is a high frequency oscillatory ventilator filtered circuit, an infant flow circuit, a precision flow circuit, a continuous positive airway pressure system circuit, a high flow breathing circuit, a high flow infant circuit nasal cannula circuit, or a high flow nasal cannula oxygen therapy circuit.

Embodiment 29: A therapeutic gas delivery system comprising: a first gas supply subsystem comprising a first compressed gas cylinder configured to supply therapeutic gas; a second gas supply subsystem comprising a second compressed gas cylinder configured to supply therapeutic gas; a therapeutic gas delivery system controller comprising a CPU, wherein the therapeutic gas delivery system receives therapeutic gas from the first compressed gas cylinder until the first compressed gas cylinder is depleted, wherein when the first compressed gas cylinder is depleted the therapeutic gas delivery system controller performs an automatic switchover such that the therapeutic gas delivery system receives therapeutic gas from the second compressed gas cylinder.

Embodiment 30: The therapeutic gas delivery system of claim 29, wherein the automatic switchover provides continuous uninterrupted delivery of therapeutic gas to a patient.

Embodiment 31: The therapeutic gas delivery system of claim 29, wherein the therapeutic gas delivery system receiving therapeutic gas from the second compressed gas cylinder provides time to disconnect the first compressed gas cylinder and connect a replacement compressed gas cylinder to the first gas supply subsystem.

Embodiment 32: The therapeutic gas delivery system of claim 31, wherein the replacement compressed gas cylinder includes a cylinder concentration such that the cylinder concentration may be verified before the replacement compressed gas cylinder is connected.

Embodiment 33: The therapeutic gas delivery system of claim 31, wherein the replacement compressed gas cylinder includes an expiration date such that the expiration date may be verified before the replacement compressed gas cylinder is connected.

Embodiment 34: The therapeutic gas delivery system of claim 29, further comprising at least one display in communication with the therapeutic gas delivery system controller, wherein the at least one display is configured to display a visual alarm when the first compressed gas cylinder is depleted.

Embodiment 35: The therapeutic gas delivery system of claim 34, wherein the at least one display is configured to display a visual alarm if the replacement compressed gas cylinder is not full with therapeutic gas.

Embodiment 36: The therapeutic gas delivery system of claim 29, wherein the therapeutic gas delivery system controller produces an audible alarm when the first compressed gas cylinder is depleted.

Embodiment 37: The therapeutic gas delivery system of claim 36, wherein the therapeutic gas delivery system controller produces an audible alarm if the replacement compressed gas cylinder is not full with therapeutic gas.

What is claimed is:

1. A therapeutic gas delivery system comprising:
at least one gas supply subsystem comprising:
a gas source coupling configured to receive a therapeutic gas source and form a fluid flow connection with the therapeutic gas source;
a therapeutic gas delivery system controller; and
one or more display(s) configured to be in communication over a communication path with the therapeutic gas delivery system controller,
wherein the one or more display(s) are configured to display a graphical, illustrative, or numerical indicator of at least one of a flow parameter and/or a system parameter.

2. The therapeutic gas delivery system of claim 1, wherein the one or more display(s) are further configured to display a visual alarm.

3. The therapeutic gas delivery system of claim 2, wherein the therapeutic gas delivery system controller is configured to produce an audible alarm.

4. The therapeutic gas delivery system of claim 1, wherein the one or more display(s) are configured to display a bar graph, a chart, or a numerical display of a value of the at least one of the flow parameter and/or the system parameter.

5. The therapeutic gas delivery system of claim 1, wherein the one or more display(s) are configured to present a graphic of the therapeutic gas delivery system comprising indicators for flow of the therapeutic gas through the system.

6. The therapeutic gas delivery system of claim 5, wherein the graphic of the therapeutic gas delivery system further comprises indicators for a status of the therapeutic gas source, a sample line, an injector module, expiratory tubing, an electronically controlled gas blending device, or combinations thereof.

7. The therapeutic gas delivery system of claim 1, the therapeutic gas is nitric oxide.

8. The therapeutic gas delivery system of claim 7, wherein the display is configured to present a concentration and/or flow rate of nitric oxide and breathing gas through the system.

9. The therapeutic gas delivery system of claim 1, further comprising an electrical power supply connection, an internal battery, and a power button, wherein the power button includes a power indicator that visually indicates an electrical power supply status of the electrical power supply connection and a battery charge status of the internal battery.

10. The therapeutic gas delivery system of claim 9, wherein one or more system parameters includes an electrical power supply status and a battery charge status.

11. The therapeutic gas delivery system of claim 1, further comprising an alarm silence button, wherein the alarm silence button includes an alarm indicator that visually indicates a status of one or more alarms of the therapeutic gas delivery system.

12. The therapeutic gas delivery system of claim 1, further comprising a global status indicator, wherein the global status indicator visually indicates one or more global statuses of the therapeutic gas delivery system.

13. The therapeutic gas delivery system of claim 12, wherein one or more of the global statuses include a delivery status and an alarm status.

14. The therapeutic gas delivery system of claim 1, further comprising an injector module outlet and a corresponding port indicator, wherein the port indicator visually indicates a delivery status of the injector module outlet.

15. The therapeutic gas delivery system of claim 1, further comprising a secondary delivery subsystem outlet and a corresponding port indicator, wherein the port indicator visually indicates a delivery status of the secondary delivery subsystem outlet.

16. The therapeutic gas delivery system of claim 1, wherein the therapeutic gas source includes a first compressed gas cylinder, wherein the one or more flow or system parameters includes a first cylinder status, wherein the first cylinder status visually indicates whether the first compressed gas cylinder is delivering therapeutic gas or is idle, wherein the first cylinder status includes a percentage of available therapeutic gas in the first compressed gas cylinder.

17. The therapeutic gas delivery system of claim 16, wherein the first cylinder status visually indicates if the first compressed gas cylinder is in a low priority alarm or a high priority alarm.

18. The therapeutic gas delivery system of claim 16, wherein the therapeutic gas source includes a second compressed gas cylinder, wherein the one or more flow or system parameters includes a second cylinder status, wherein the second cylinder status visually indicates whether the second compressed gas cylinder is delivering therapeutic gas or is idle, wherein the second cylinder status includes a percentage of available therapeutic gas in the second compressed gas cylinder.

19. The therapeutic gas delivery system of claim 18, wherein the second cylinder status visually indicates if the second compressed gas cylinder is in a low priority alarm or a high priority alarm.

20. The therapeutic gas delivery system of claim 1, wherein the one or more flow or system parameters includes at least one of a left cylinder status, right cylinder status, a patient line status, a sample gas sample line status, a delivery conduit status, an injector module status, a patient gas sample line status, and a breathing circuit expiratory limb status.

21. The therapeutic gas delivery system of claim 1, wherein the one or more flow or system parameters includes at least one of a secondary delivery subsystem status and dose.

22. The therapeutic gas delivery system of claim 1, wherein at least one gas supply system includes a secondary delivery subsystem, wherein the one or more displays is configured to display the delivery of therapeutic gas through the secondary delivery subsystem.

23. The therapeutic gas delivery system of claim 22, further comprising a breathing circuit, wherein the one or more displays is configured to display the delivery of therapeutic gas to the breathing circuit.

24. The therapeutic gas delivery system of claim 23, wherein the breathing circuit is a t-piece resuscitator circuit, an infant resuscitator circuit, or an infant t-piece resuscitator circuit.

25. The therapeutic gas delivery system of claim 1, wherein at least one gas supply system includes a therapeutic gas delivery system, wherein the one or more displays is configured to display the delivery of therapeutic gas through the therapeutic gas delivery system.

26. The therapeutic gas delivery system of claim 25, further comprising a breathing circuit, wherein the one or more displays is configured to display the delivery of therapeutic gas to the breathing circuit.

27. The therapeutic gas delivery system of claim 26, wherein the breathing circuit is a conventional ventilator circuit, an anesthesia circuit, a spontaneous circuit, a nasal cannula circuit, a high frequency jet ventilation circuit, a high frequency oscillatory ventilator unfiltered circuit, a dual-limb transport ventilator circuit, or a single-limb transport ventilator circuit.

28. The therapeutic gas delivery system of claim 26, wherein the breathing circuit is a high frequency oscillatory ventilator filtered circuit, an infant flow circuit, a precision flow circuit, a continuous positive airway pressure system circuit, a high flow breathing circuit, a high flow infant circuit nasal cannula circuit, or a high flow nasal cannula oxygen therapy circuit.

29. A therapeutic gas delivery system comprising:
a first gas supply subsystem comprising a first compressed gas cylinder configured to supply therapeutic gas;
a second gas supply subsystem comprising a second compressed gas cylinder configured to supply therapeutic gas; and
a therapeutic gas delivery system controller,
wherein the therapeutic gas delivery system receives therapeutic gas from the first compressed gas cylinder until the first compressed gas cylinder is depleted, wherein when the first compressed gas cylinder is depleted the therapeutic gas delivery system controller performs an automatic switchover such that the therapeutic gas delivery system receives therapeutic gas from the second compressed gas cylinder.

30. The therapeutic gas delivery system of claim 29, wherein the automatic switchover provides continuous uninterrupted delivery of therapeutic gas to a patient.

31. The therapeutic gas delivery system of claim 29, wherein the therapeutic gas delivery system receiving therapeutic gas from the second compressed gas cylinder provides time to disconnect the first compressed gas cylinder and connect a replacement compressed gas cylinder to the first gas supply subsystem.

32. The therapeutic gas delivery system of claim 31, wherein the replacement compressed gas cylinder includes a cylinder concentration such that the cylinder concentration can be verified before the replacement compressed gas cylinder is connected.

33. The therapeutic gas delivery system of claim 31, wherein the replacement compressed gas cylinder includes an expiration date such that the expiration date can be verified before the replacement compressed gas cylinder is connected.

34. The therapeutic gas delivery system of claim 29, further comprising at least one display in communication with the therapeutic gas delivery system controller, wherein the at least one display is configured to display a visual alarm when the first compressed gas cylinder is depleted.

35. The therapeutic gas delivery system of claim 34, wherein the at least one display is configured to display a visual alarm if a replacement compressed gas cylinder is not full with therapeutic gas.

36. The therapeutic gas delivery system of claim 29, wherein the therapeutic gas delivery system controller produces an audible alarm when the first compressed gas cylinder is depleted.

37. The therapeutic gas delivery system of claim 36, wherein the therapeutic gas delivery system controller produces an audible alarm if a replacement compressed gas cylinder is not full of therapeutic gas.
